(19) United States
(12) Patent Application Publication
(10) Pub. No.: US 2025/0381185 A1
(43) Pub. Date: Dec. 18, 2025

(54) CDK4/PARP1 DUAL-TARGET INHIBITOR AND USE THEREOF

(71) Applicant: YANBIAN UNIVERSITY, YANBIAN (CN)

(72) Inventors: QINGKUN SHEN, YANBIAN (CN); XIAOTING LI, YANBIAN (CN); YALAN WANG, YANBIAN (CN); MIAOXIA PU, YANBIAN (CN)

(73) Assignee: YANBIAN UNIVERSITY, YANBIAN (CN)

(21) Appl. No.: 19/233,062

(22) Filed: Jun. 10, 2025

(30) Foreign Application Priority Data
Jun. 13, 2024 (CN) 2024107628918
May 29, 2025 (CN) 2025107077498

(51) Int. Cl. A61K 31/506; A61K 31/519

(52) U.S. Cl. CPC A61K 31/506; A61K 31/519

(57) ABSTRACT

The present application discloses a CDK4/PARP1 dual-target inhibitor and use thereof, which belongs to the field of medical technologies and solves the problems of poor efficacy of single CDK4 inhibitors and weak activity of single PARP1 inhibitors. The CDK4/PARP1 dual-target inhibitor is a compound having a general structural formula A-Linker-B, or a pharmaceutically acceptable salt, solvate, hydrate, or prodrug thereof. The CDK4/PARP1 dual-target inhibitor of the present application effectively overcomes the shortcomings of poor efficacy of single CDK4 inhibitors and weak activity of single PARP1 inhibitors, exhibiting strong inhibitory activity against both CDK4 and PARP1, and having promising application prospects.

### TECHNICAL FIELD

The present application belongs to the field of medical technologies, and specifically relates to a CDK4/PARP1 dual-target inhibitor and use thereof.

### BACKGROUND

The malignant proliferation of tumor cells is controlled by a complex interactive signal network, and the molecular mechanism of cell carcinogenesis has not yet been fully elucidated. Intervention on only one of the targets often fails to completely inhibit tumor cell proliferation due to the compensatory mechanism of the signal network, and is therefore prone to drug resistance. Therefore, in recent years, it has been found that most single-target inhibitors are not effective in treating malignant tumors, and long-term use can easily lead to insensitivity and drug resistance. To solve this problem, there are two strategies. One is combination therapy, that is, the combined use of multiple drugs targeting different targets. However, although the combination therapy has a high anti-tumor efficacy, it is also associated with poor patient compliance, increased adverse effects, unpredictable pharmacokinetic characteristics, and drug interactions, which seriously affect the efficacy of one or more of the drugs. The other is to incorporate two or more drugs or pharmacophores acting on different targets into the same molecule to design a multi-target tumor inhibitor. It can simultaneously act on multiple molecular targets that control tumor cell proliferation, metabolism, apoptosis, and metastasis, can inhibit different cellular pathways or compensatory mechanisms, and has a wide range of biological activities. It can not only overcome the drug resistance caused by long-term use of single-target drugs, but also avoid drug interactions. Therefore, the development of multi-target drugs is considered a promising and effective way for discovery of new anti-cancer drugs.

Cyclin-dependent kinases (CDKs) are key regulators of cell growth and division, playing a key role in cell cycle control and transcriptional regulation. Ribociclib is a marketed CDK4/6 inhibitor, with IC₅₀ values of 10 nM and 39 nM for CDK4 and CDK6 respectively. Molecular docking analysis of Ribociclib with CDK4 (PDB: 7SJ3) shows that the 7H-pyrrolo[2,3-d]pyrimidine moiety binds to the catalytic site of the CDK4 enzyme and forms a hydrogen bond interaction with Val 96, which is the key pharmacophore of Ribociclib. The pyridine-3-piperazine moiety is located in a solvent-exposed region of the CDK4 enzyme and amenable to structural modification. Poly ADP-ribose polymerase (PARP) is a DNA repair enzyme that plays a critical role in cellular response to DNA damage. Olaparib is an approved PARP inhibitor. Molecular docking analysis of Olaparib with PARP1 (PDB: 5DS3) shows that the phthalazinone moiety binds to the catalytic site of PARP1 and forms hydrogen bond interactions with Ser 904 and Gly 863, as well as n-T interactions with Tyr 907 and Tyr 896, serving as the key pharmacophore of Olaparib. The cyclopropylpiperazinone moiety is located in a solvent-exposed region of PARP1 and is amenable to structural modification. In addition, studies have shown that cell cycle progression regulates the transcription of PARP1 via the growth factor/inhibitor-G1/G0-CDK4/6-RBs axis, and dual-target inhibition of CDK4/6 and PARP1 can synergistically enhance anti-tumor activity and delay the development of drug resistance. Therefore, the development of a dual-target inhibitor that can simultaneously inhibit CDK4/6 and PARP1 will provide a new, safe and effective anti-tumor drug.

### SUMMARY

An objective of the present application is to address deficiencies of the prior art and provide a CDK4/PARP1 dual-target inhibitor and use thereof, which can effectively overcome the shortcomings of poor efficacy of single CDK4 inhibitors and weak activity of single PARP1 inhibitors, and exhibit strong inhibitory activity against both CDK4 and PARP1.

The present application is achieved by providing a CDK4/PARP1 dual-target inhibitor. The inhibitor is a compound having a general structural formula as shown in Formula 1, or a pharmaceutically acceptable salt, solvate, hydrate or prodrug thereof,

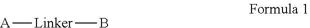

wherein A of the compound may be selected from:

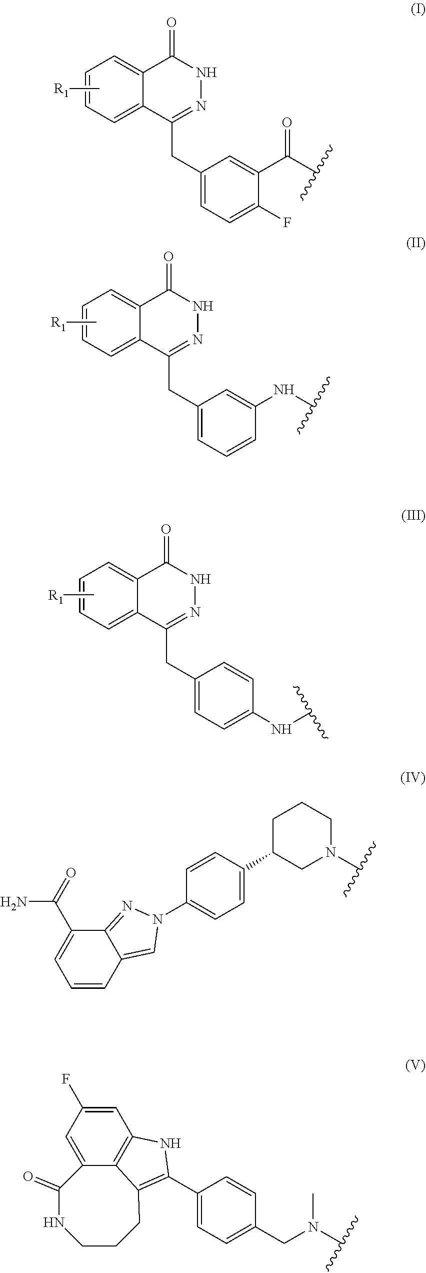

wherein R₁ is selected from H, 1-F, 2-F, 3-F, 6-F, 1-Cl, 2-Cl, 3-Cl, 6-Cl, 1-Br, 2-Br, 3-Br, 6-Br, 1-I, 2-I, 3-I, 6-I, 1-I, 2-I, 3-I, 6-I, 1-NO₂, 2-NO₂, 3-NO₂, 6-NO₂, 1-CH₃, 2-CH₃, 3-CH₃, 6-CH₃, 1-OCH₃, 2-OCH₃, 3-OCH₃, and 6-OCH₃;
when A is selected from (I), Linker is selected from the following groups:

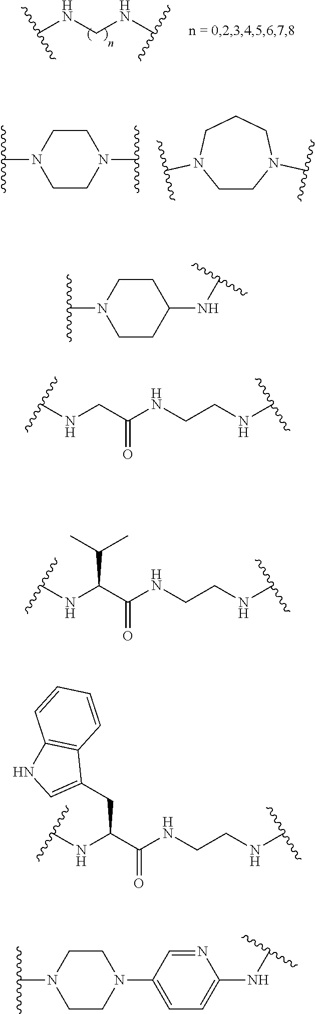

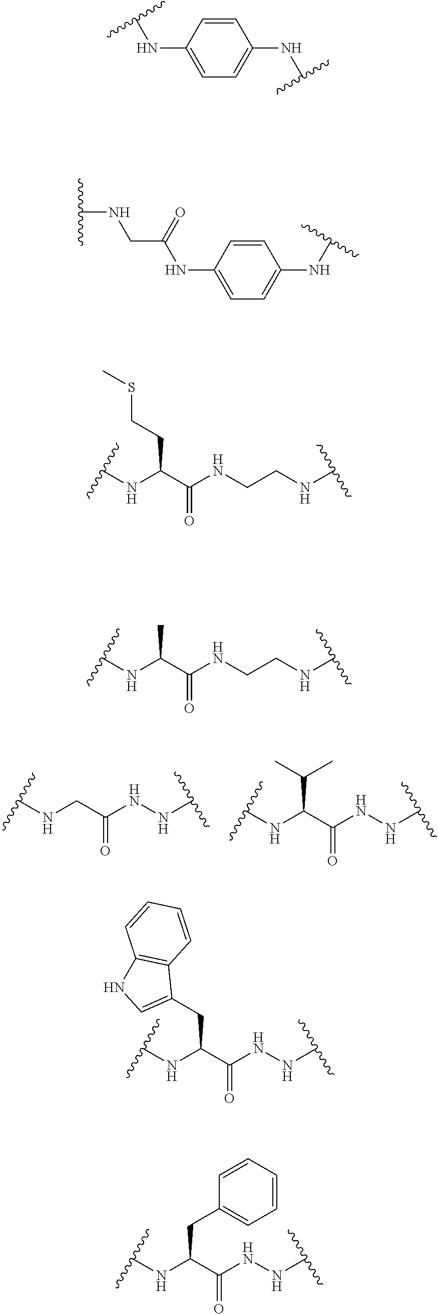

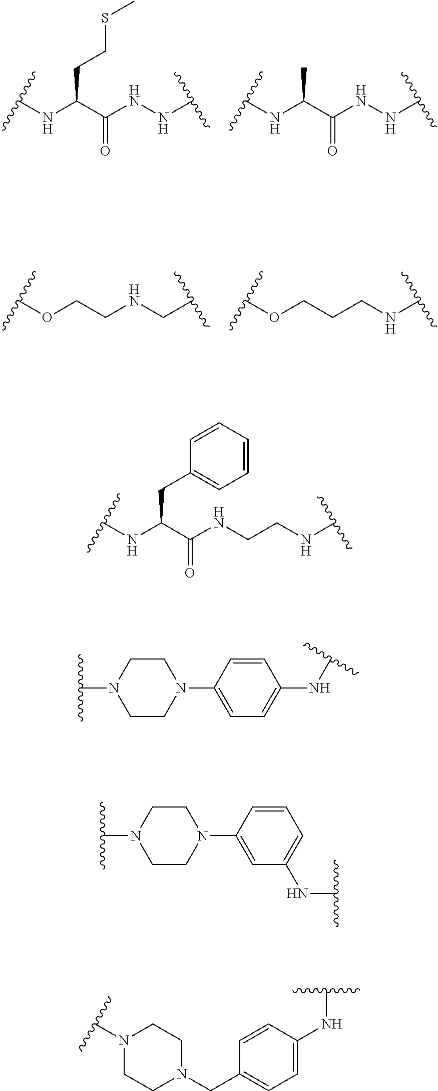

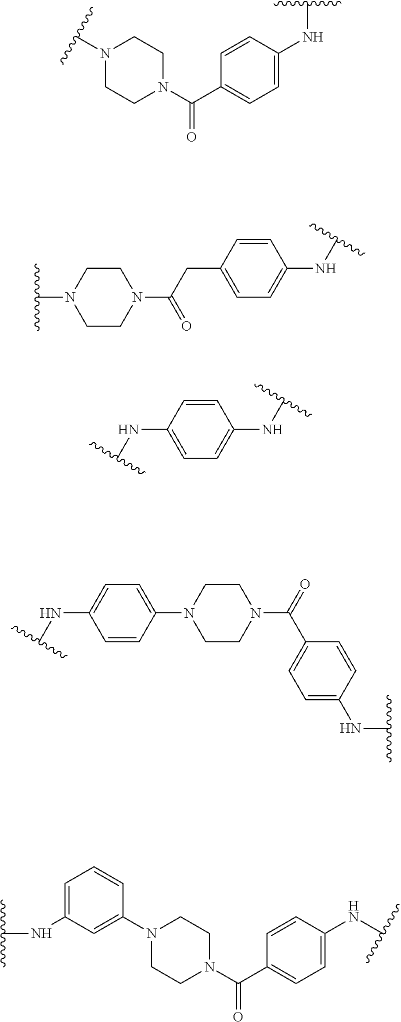 [SMILES: *Nc1ccc(C(=O)N2CCN(*)CC2)cc1 ; *Nc1ccc(CC(=O)N2CCN(*)CC2)cc1 ; *Nc1ccc(N*)cc1 ; *Nc1ccc(C(=O)N2CCN(c3ccc(N*)cc3)CC2)cc1 ; *Nc1ccc(C(=O)N2CCN(c3cccc(N*)c3)CC2)cc1]

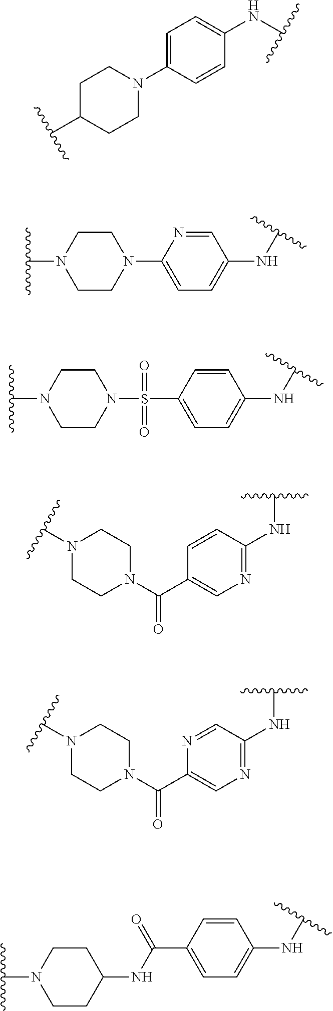 [SMILES: *Nc1ccc(N2CCC(*)CC2)cc1 ; *Nc1ccc(N2CCN(*)CC2)nc1 ; *Nc1ccc(S(=O)(=O)N2CCN(*)CC2)cc1 ; *Nc1ccc(C(=O)N2CCN(*)CC2)cn1 ; *Nc1cnc(C(=O)N2CCN(*)CC2)cn1 ; *Nc1ccc(C(=O)NC2CCN(*)CC2)cc1]

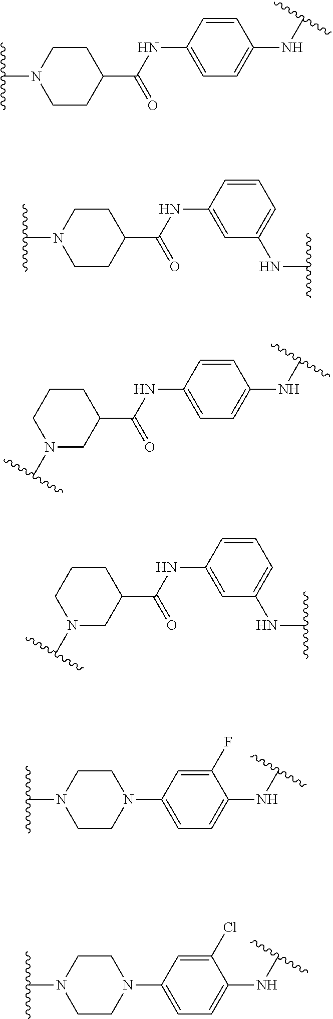 [SMILES: *Nc1ccc(NC(=O)C2CCN(*)CC2)cc1 ; *Nc1cccc(NC(=O)C2CCN(*)CC2)c1 ; *Nc1ccc(NC(=O)C2CCCN(*)C2)cc1 ; *Nc1cccc(NC(=O)C2CCCN(*)C2)c1 ; *Nc1ccc(N2CCN(*)CC2)cc1F ; *Nc1ccc(N2CCN(*)CC2)cc1Cl]

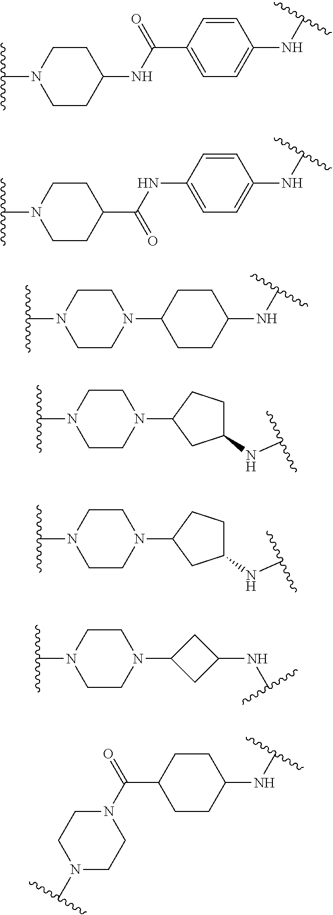

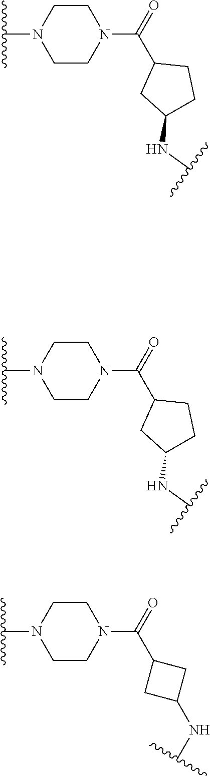 [SMILES: *N[C@@H]1CCC(C(=O)N2CCN(*)CC2)C1 ; *N[C@H]1CCC(C(=O)N2CCN(*)CC2)C1 ; *NC1CC(C(=O)N2CCN(*)CC2)C1]

when A is selected from (II), (III), (IV), and (V), Linker is selected from the following groups:

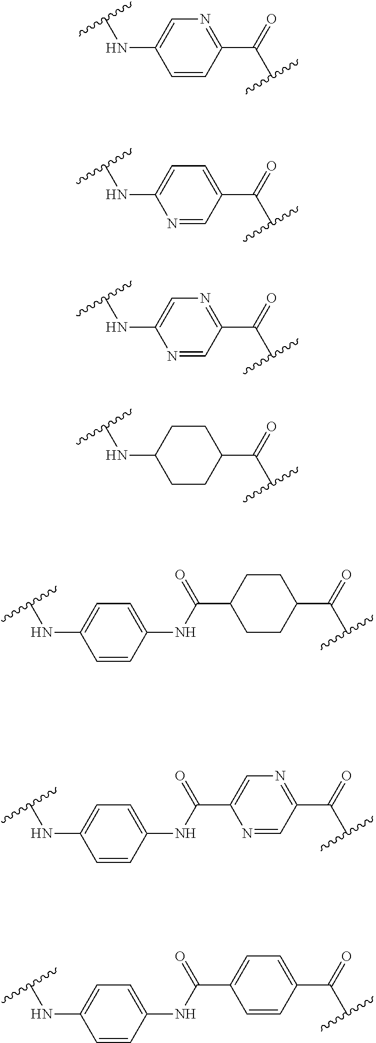

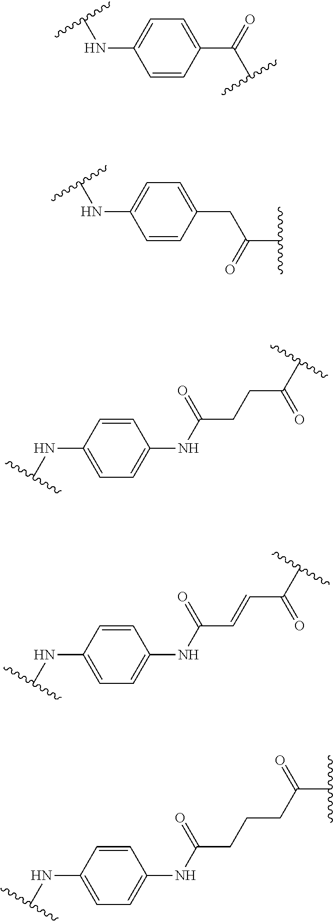 [SMILES: *Nc1ccc(C(*)=O)cc1 ; *Nc1ccc(CC(*)=O)cc1 ; *Nc1ccc(NC(=O)CCC(*)=O)cc1 ; *Nc1ccc(NC(=O)/C=C/C(*)=O)cc1 ; *Nc1ccc(NC(=O)CCCC(*)=O)cc1]

wherein B of the compound may be selected from the following groups:

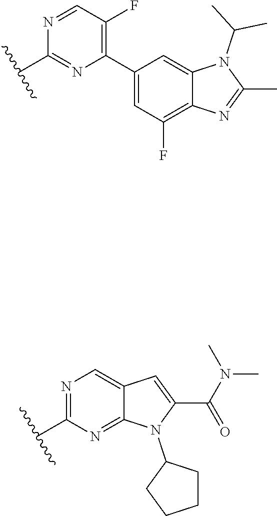 [SMILES: *c1ncc(F)c(-c2cc(F)c3nc(C)n(C(C)C)c3c2)n1 ; *c1ncc2cc(C(=O)N(C)C)n(C3CCCC3)c2n1]

Further, the inhibitor is an enantiomer, a mixture of enantiomers, a mixture of two or more diastereomers, a tautomer, a mixture of two or more tautomers, or an isotopically labeled variant of formula 1.

A dual-target inhibitor is the compound represented by any one of the following chemical structures:

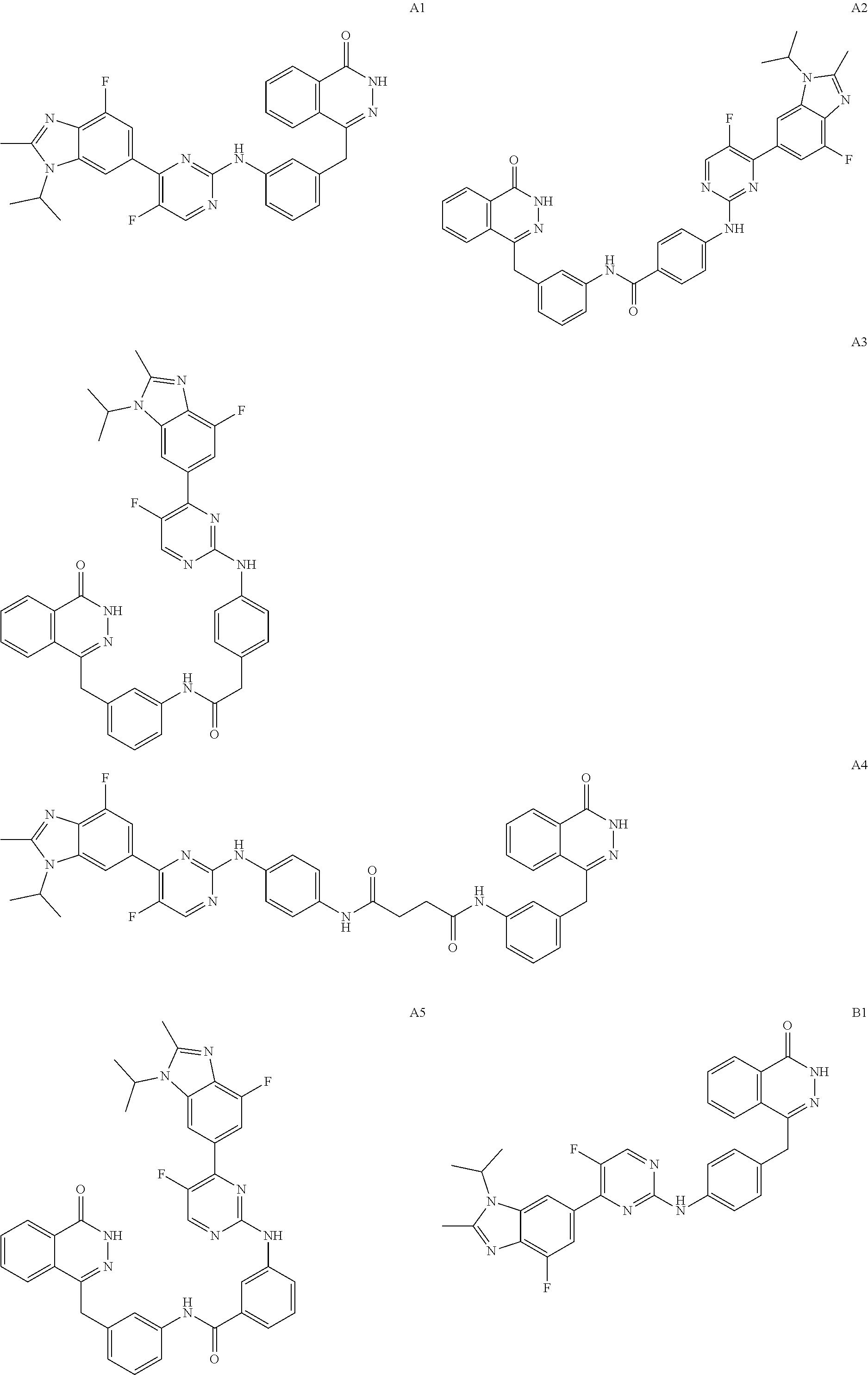

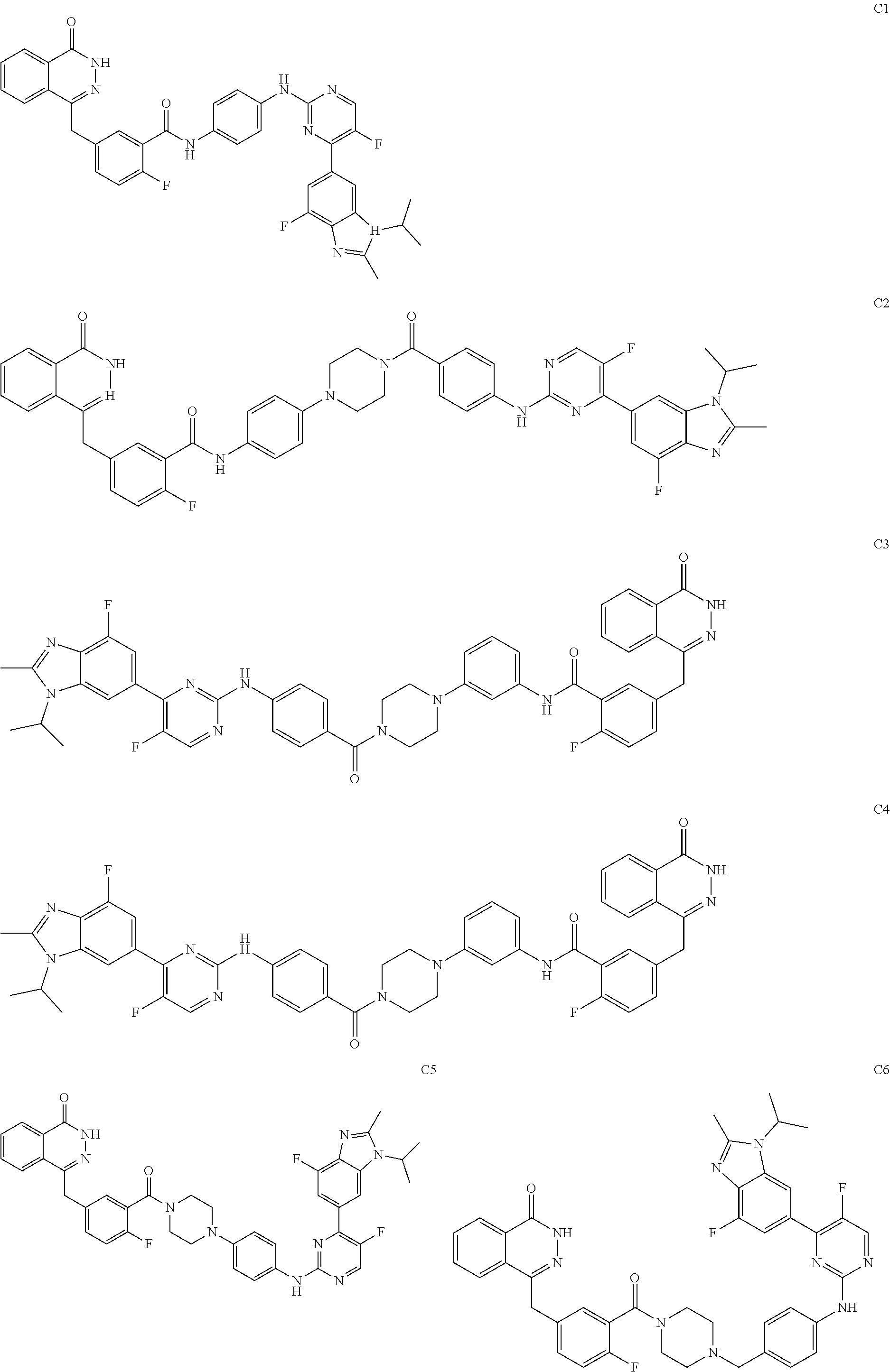

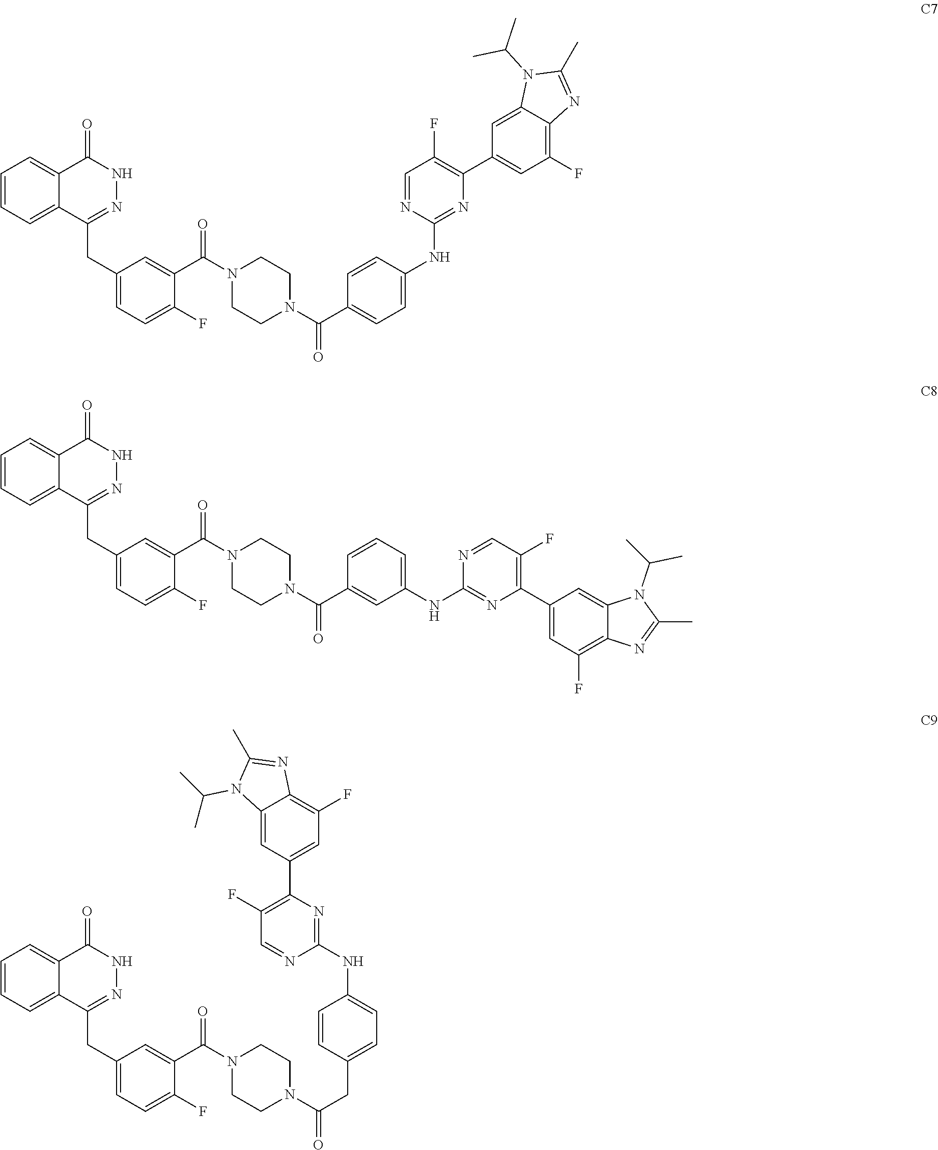 [SMILES: Cc1nc2c(F)cc(-c3nc(Nc4ccc(C(=O)N5CCN(C(=O)c6cc(Cc7n[nH]c(=O)c8ccccc78)ccc6F)CC5)cc4)ncc3F)cc2n1C(C)C ; Cc1nc2c(F)cc(-c3nc(Nc4cccc(C(=O)N5CCN(C(=O)c6cc(Cc7n[nH]c(=O)c8ccccc78)ccc6F)CC5)c4)ncc3F)cc2n1C(C)C ; Cc1nc2c(F)cc(-c3nc(Nc4ccc(CC(=O)N5CCN(C(=O)c6cc(Cc7n[nH]c(=O)c8ccccc78)ccc6F)CC5)cc4)ncc3F)cc2n1C(C)C]

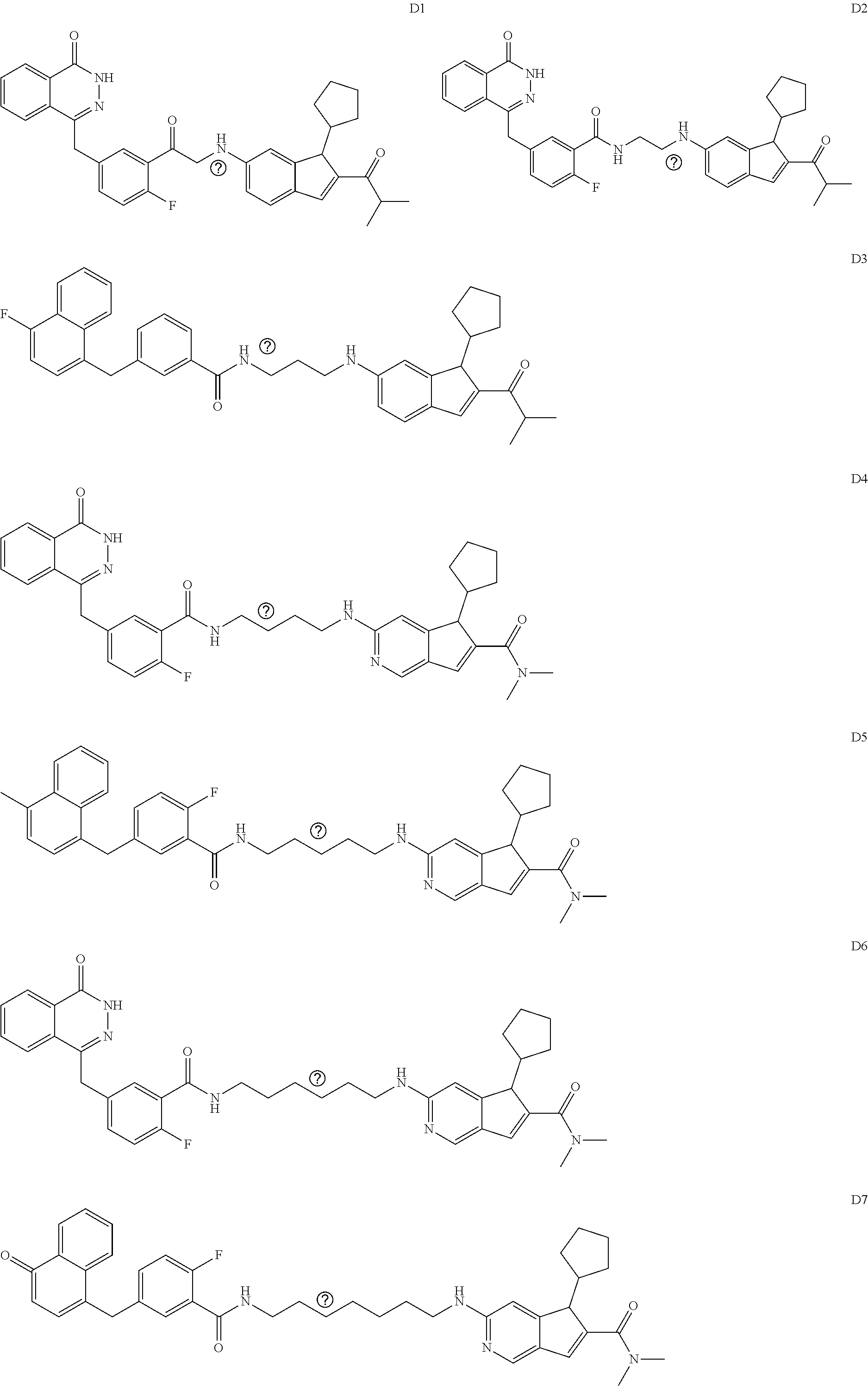

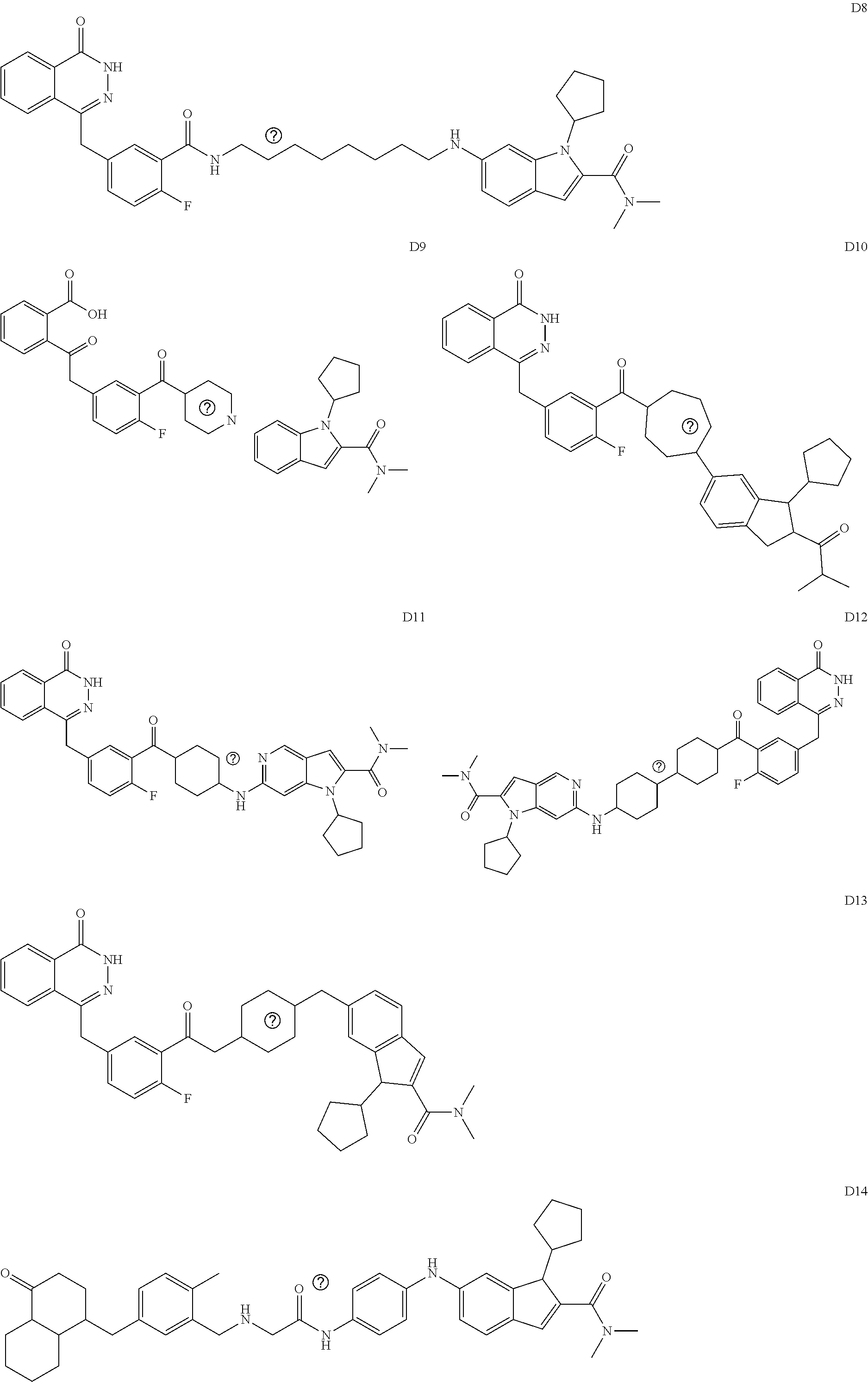

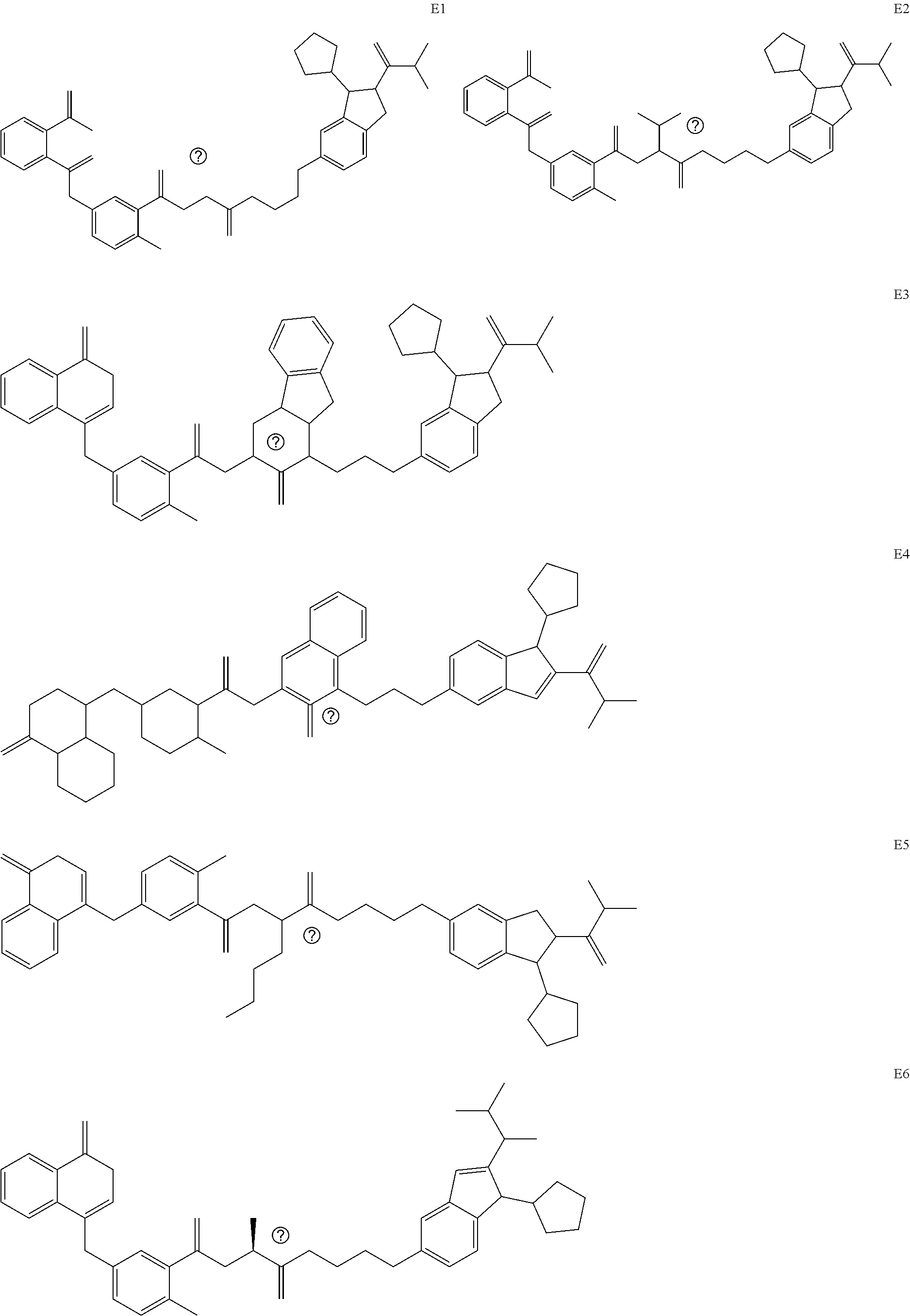

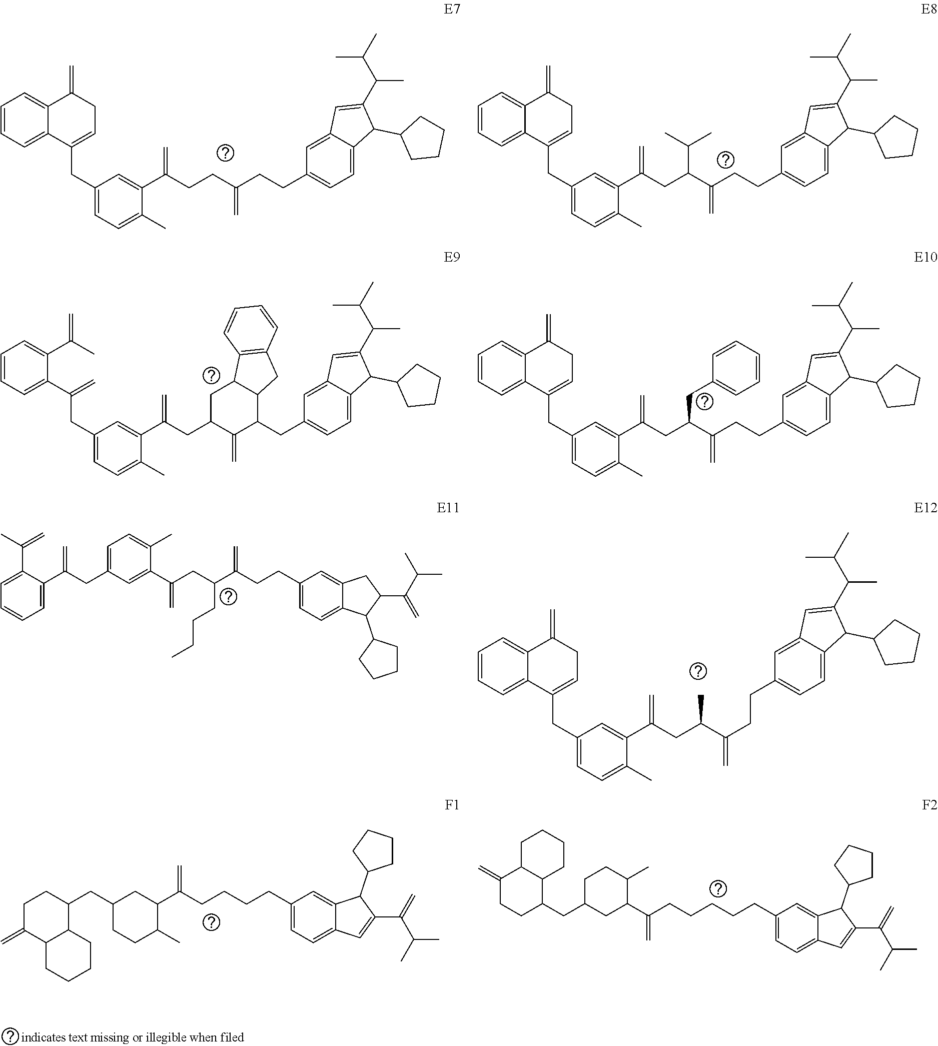

The above are some specific structural forms of the dual-target inhibitor, but are not limited to the chemical structures listed herein. All compounds based on the general structural formula of formula 1, wherein A, B, and Linker are simple modifications of any groups previously defined within the scope, are intended to be included.

The present application proposes use of the dual-target inhibitor for simultaneously inhibiting CDK4, CDK6, and PARP1 activities.

The present application further proposes use of the dual-target inhibitor in preparation of a medicament for treating, ameliorating or preventing diseases mediated by poly(ADP-ribose) polymerase (PARP), cyclin-dependent kinase 4 (CDK4), and cyclin-dependent kinase 6 (CDK6).

Furthermore, the dual-target inhibitor described above can be used to prepare an anti-tumor drug.

A pharmaceutical composition includes one or more dual-target inhibitors described above.

A pharmaceutical composition containing the dual-target inhibitor includes the inhibitor as an active ingredient and a pharmaceutically acceptable carrier.

A pharmaceutical preparation includes a therapeutically effective amount of dual-target inhibitor described above and a pharmaceutically acceptable excipient.

The pharmaceutical preparation includes the following dosage forms: oral preparations (such as tablets, capsules, solutions or suspensions); injectable preparations (such as injectable solutions or suspensions, or injectable dry powders, which can be used immediately by adding injection water before injection) and topical preparations (such as ointments or solutions).

The carrier used in the pharmaceutical composition of the present application is a common carrier available in the pharmaceutical field, including: for injectable preparations: binders, lubricants, disintegrants, solubilizers, diluents, stabilizers, suspending agents, being colorant-free, and flavoring agents; for injectable preparations: preservatives, solubilizing agent, and stabilizer; and for topical preparations: bases, diluents, lubricants, and preservatives. The pharmaceutical preparations can be administered orally or parenterally (e.g., intravenously, subcutaneously, intraperitoneally or topically), and if active ingredients are unstable under gastric conditions, they can be formulated into enteric-coated tablets.

The pharmaceutical preparation is used for treating one or more symptoms of a disorder, disease or condition mediated by PARP and CDK4/6, including administering the pharmaceutical preparation to a human or mammal in need thereof; and the disorder, disease or condition mediated by PARP and CDK4/6 is cancer.

Compared with the prior art, examples of the present application have the following beneficial effects.

The dual-target inhibitor described in the present application can effectively overcome the shortcomings of poor efficacy of single CDK4 inhibitors and weak activity of single PARP1 inhibitors, and exhibits strong inhibitory effects on both CDK4 and PARP1, and has good application prospects.

The dual-target inhibitor of the present application has a simple preparation process, high purity, high yield, and stable quality, and is suitable for large-scale production.

### DETAILED DESCRIPTION OF PREFERRED EMBODIMENTS

Unless otherwise defined, all technical and scientific terms used herein have the same meaning as those commonly understood by technicians in the technical field of this application; the terms used in the specification of the application herein are only for the purpose of describing specific embodiments and are not intended to limit the present application; and the terms “including” and “having” and any variations thereof in the specification, claims and drawings of the application are intended to cover non-exclusive inclusions. Terms “first”, “second”, and the like in the specification, claims, and drawings of the application are adopted not to describe a specific sequence but to distinguish different objects. All raw materials used in the present application are commercially available products in the art, unless otherwise specified.

Embodiments of the present application provide a CDK4/PARP1 dual-target inhibitor and use thereof. The inhibitor is a compound having a general structural formula as shown in formula (I), or a pharmaceutically acceptable salt, solvate, hydrate, or prodrug thereof. The CDK4/PARP1 dual-target inhibitor of the present application effectively overcomes the shortcomings of poor efficacy of single CDK4 inhibitors and weak activity of single PARP1 inhibitors, exhibiting strong inhibitory activity against both CDK4 and PARP1, and having promising application prospects.

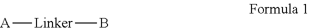

wherein A of the compound may be selected from:

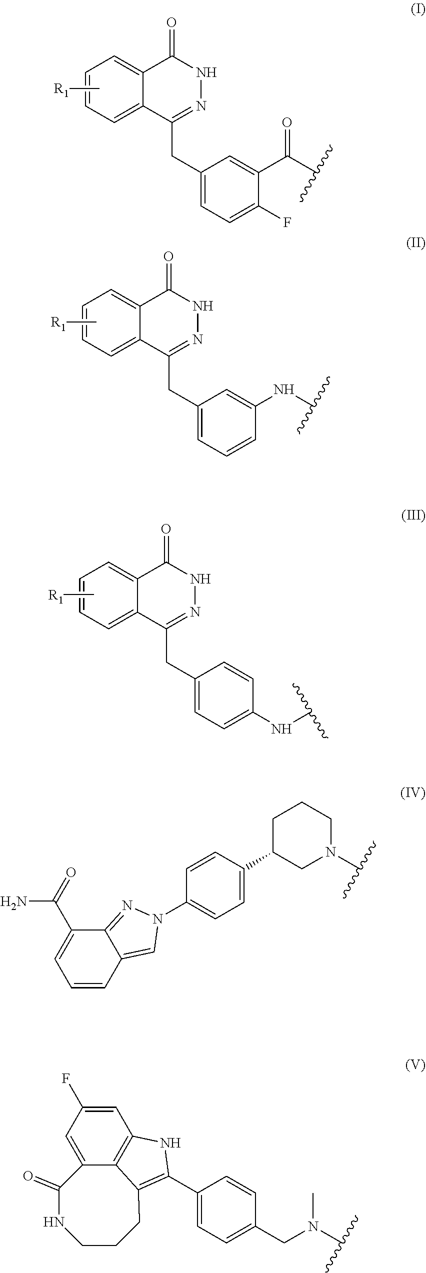

wherein R₁ is selected from H, 1-F, 2-F, 3-F, 6-F, 1-Cl, 2-Cl, 3-Cl, 6-Cl, 1-Br, 2-Br, 3-Br, 6-Br, 1-I, 2-I, 3-I, 6-I, 1-I, 2-I, 3-I, 6-I, 1-NO₂, 2-NO₂, 3-NO₂, 6-NO₂, 1-CH₃, 2-CH₃, 3-CH₃, 6-CH₃, 1-OCH₃, 2-OCH₃, 3-OCH₃, and 6-OCH₃; and
when A is selected from (I), Linker is selected from the following groups:

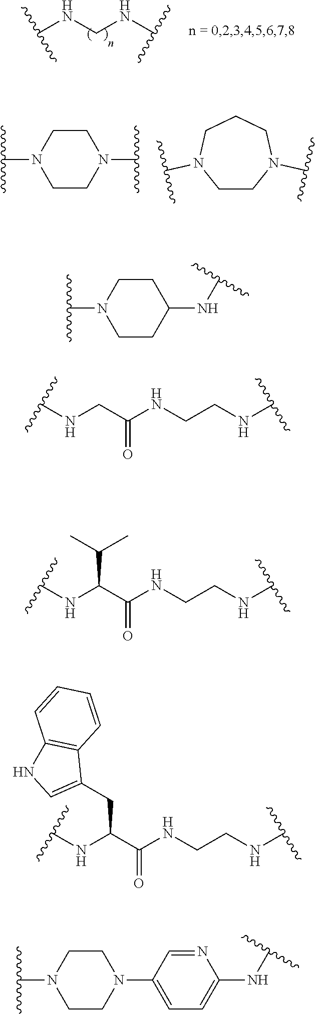

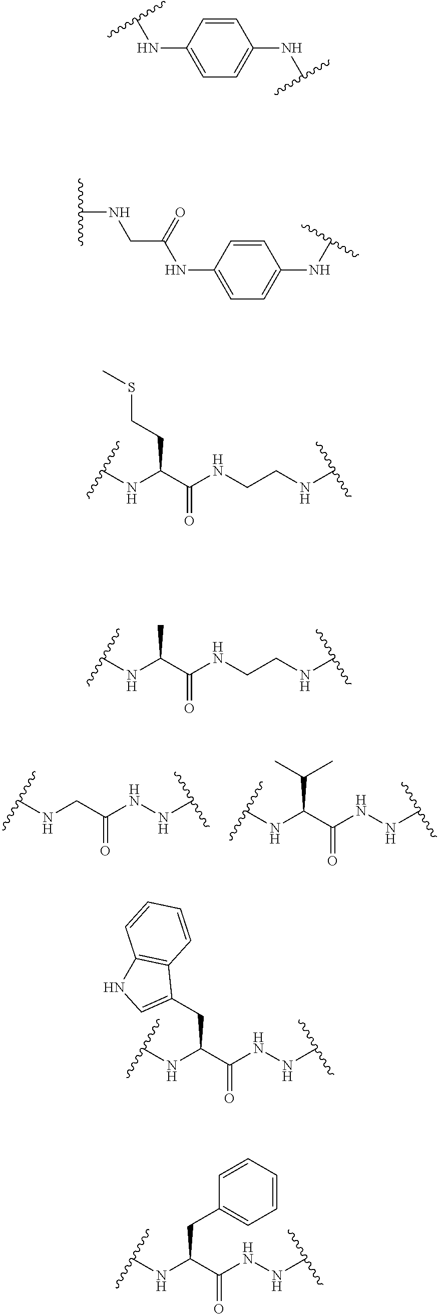

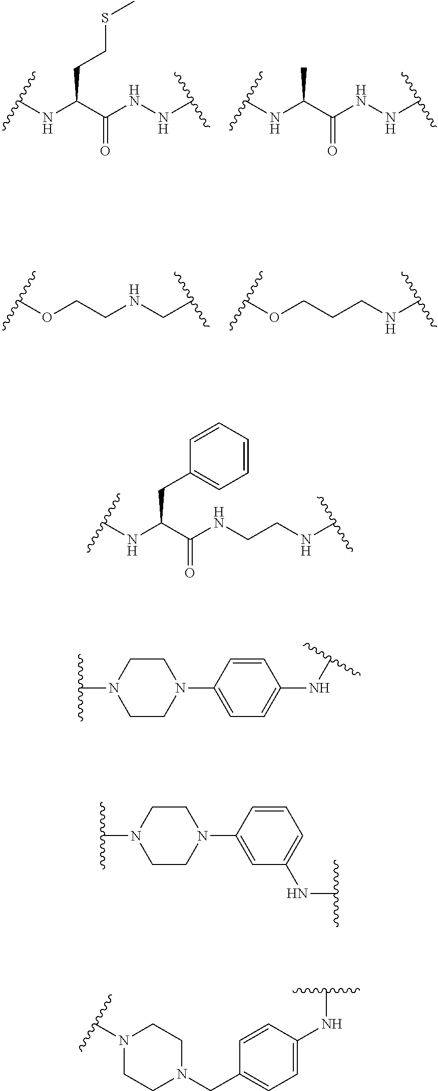

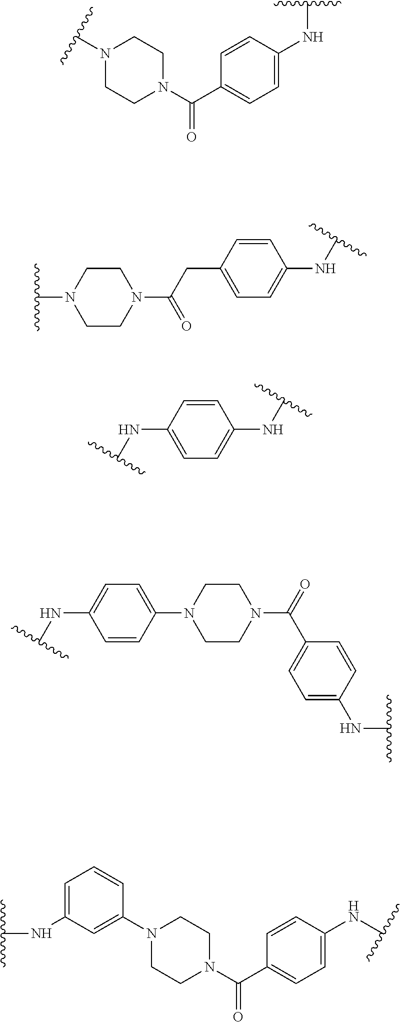 [SMILES: *Nc1ccc(C(=O)N2CCN(*)CC2)cc1 ; *Nc1ccc(CC(=O)N2CCN(*)CC2)cc1 ; *Nc1ccc(N*)cc1 ; *Nc1ccc(C(=O)N2CCN(c3ccc(N*)cc3)CC2)cc1 ; *Nc1ccc(C(=O)N2CCN(c3cccc(N*)c3)CC2)cc1]

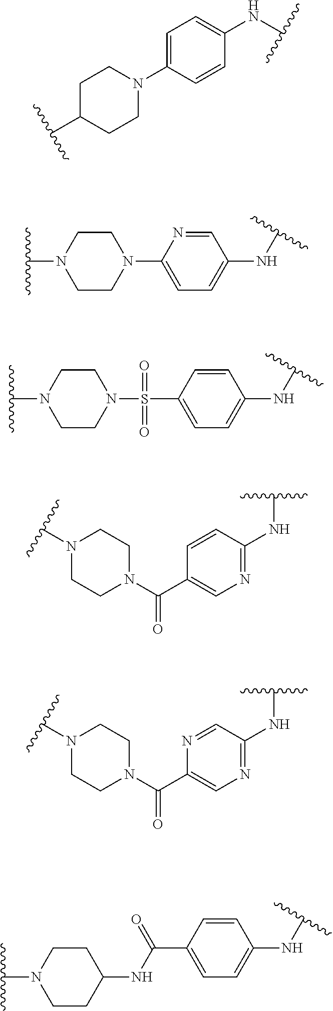 [SMILES: *Nc1ccc(N2CCC(*)CC2)cc1 ; *Nc1ccc(N2CCN(*)CC2)nc1 ; *Nc1ccc(S(=O)(=O)N2CCN(*)CC2)cc1 ; *Nc1ccc(C(=O)N2CCN(*)CC2)cn1 ; *Nc1cnc(C(=O)N2CCN(*)CC2)cn1 ; *Nc1ccc(C(=O)NC2CCN(*)CC2)cc1]

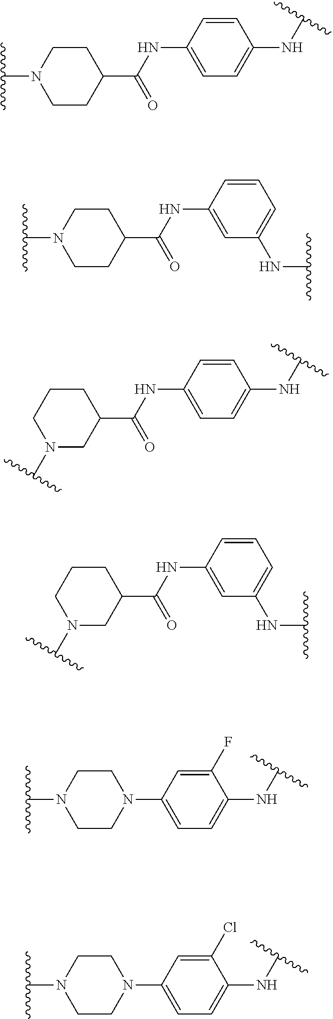 [SMILES: *Nc1ccc(NC(=O)C2CCN(*)CC2)cc1 ; *Nc1cccc(NC(=O)C2CCN(*)CC2)c1 ; *Nc1ccc(NC(=O)C2CCCN(*)C2)cc1 ; *Nc1cccc(NC(=O)C2CCCN(*)C2)c1 ; *Nc1ccc(N2CCN(*)CC2)cc1F ; *Nc1ccc(N2CCN(*)CC2)cc1Cl]

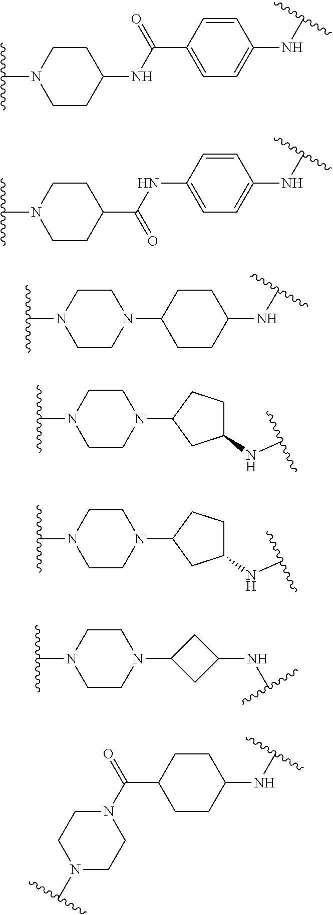

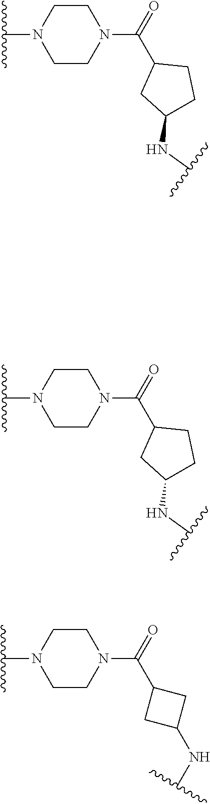 [SMILES: *N[C@@H]1CCC(C(=O)N2CCN(*)CC2)C1 ; *N[C@H]1CCC(C(=O)N2CCN(*)CC2)C1 ; *NC1CC(C(=O)N2CCN(*)CC2)C1]

when A is selected from (II), (III), (IV), and (V), Linker is selected from the following groups:

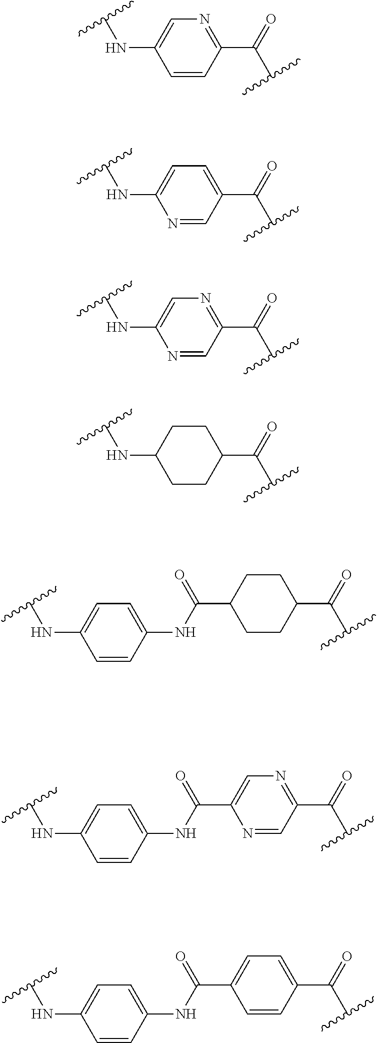

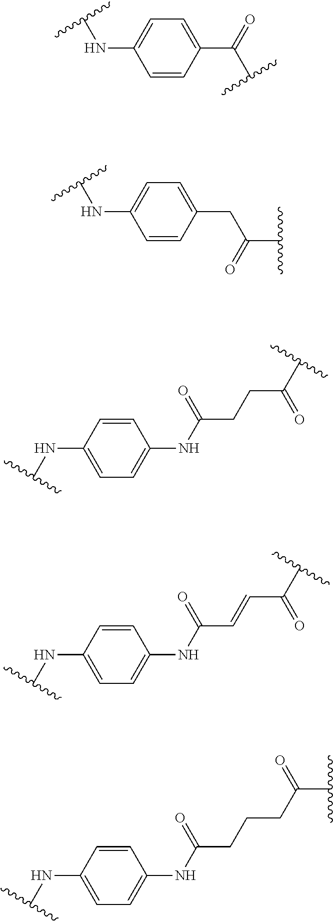 [SMILES: *Nc1ccc(C(*)=O)cc1 ; *Nc1ccc(CC(*)=O)cc1 ; *Nc1ccc(NC(=O)CCC(*)=O)cc1 ; *Nc1ccc(NC(=O)/C=C/C(*)=O)cc1 ; *Nc1ccc(NC(=O)CCCC(*)=O)cc1]

wherein B of the compound may be selected from the following groups:

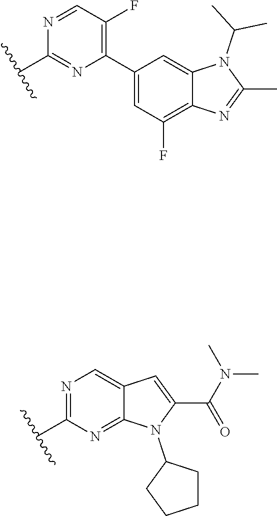 [SMILES: *c1ncc(F)c(-c2cc(F)c3nc(C)n(C(C)C)c3c2)n1 ; *c1ncc2cc(C(=O)N(C)C)n(C3CCCC3)c2n1]

### Example 1

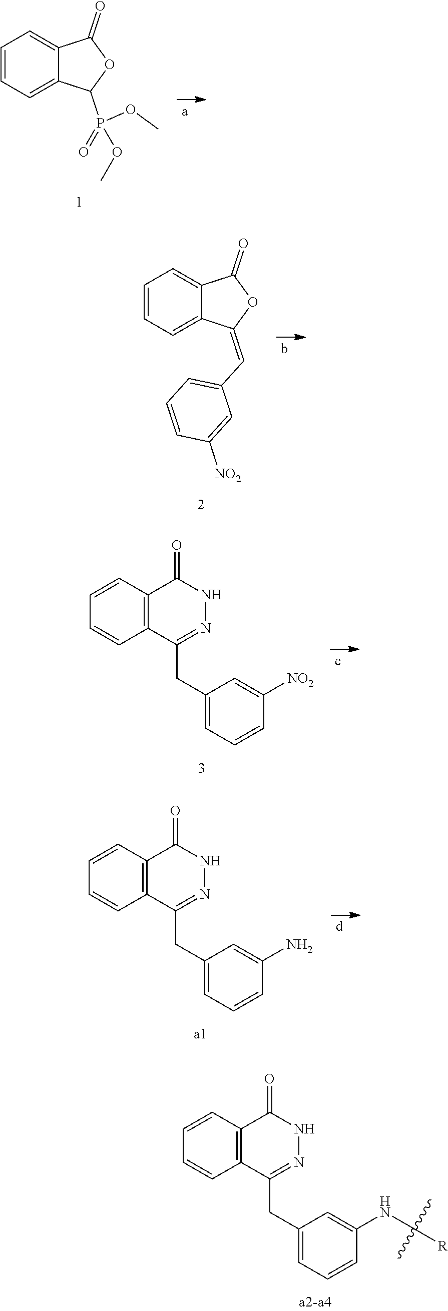

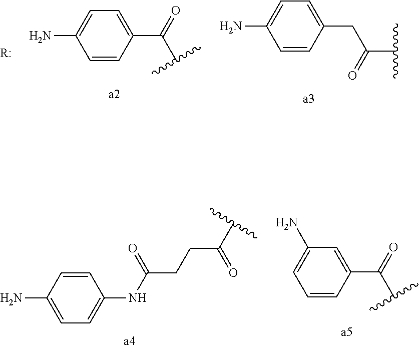

Synthesis of intermediate 2: Compound 1 (1 mmol) and m-nitrobenzaldehyde (1 mmol) were dissolved in 50 mL of tetrahydrofuran, and 5 mL of triethylamine was added. The mixture was reacted at room temperature overnight. The reaction was monitored by thin-layer chromatography (TLC) and confirmed to be complete. After completion of the reaction, the reaction mixture was poured into a saturated sodium chloride solution and extracted with dichloromethane. An organic phase was collected, dried over anhydrous Na₂SO₄, and concentrated under reduced pressure. A crude product was purified by column chromatography to yield the intermediate 2.

Synthesis of intermediate 3: Compound 2 (1 mmol) and hydrazine hydrate (1 mmol) were dissolved in 50 mL of solvent mixture of ethanol and water (10:1). Sodium hydroxide (1 mmol) was added, and the mixture was reacted at 90 C overnight. The reaction was monitored by TLC and confirmed to be complete. After completion of the reaction, the reaction mixture was then poured into a saturated sodium chloride solution and extracted with dichloromethane. An organic phase was collected, dried over anhydrous Na₂SO₄, and concentrated under reduced pressure. A crude product was purified by column chromatography to yield the intermediate 3.

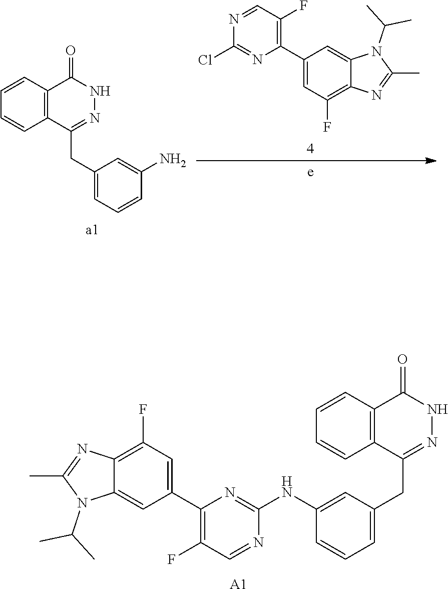

Synthesis of target compound A1: Compound a1 (0.05 mmol) and compound 4 (0.05 mmol) were dissolved in 50 mL of n-butanol. A catalytic amount of concentrated hydrochloric acid was added, and the mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the target compound A1.

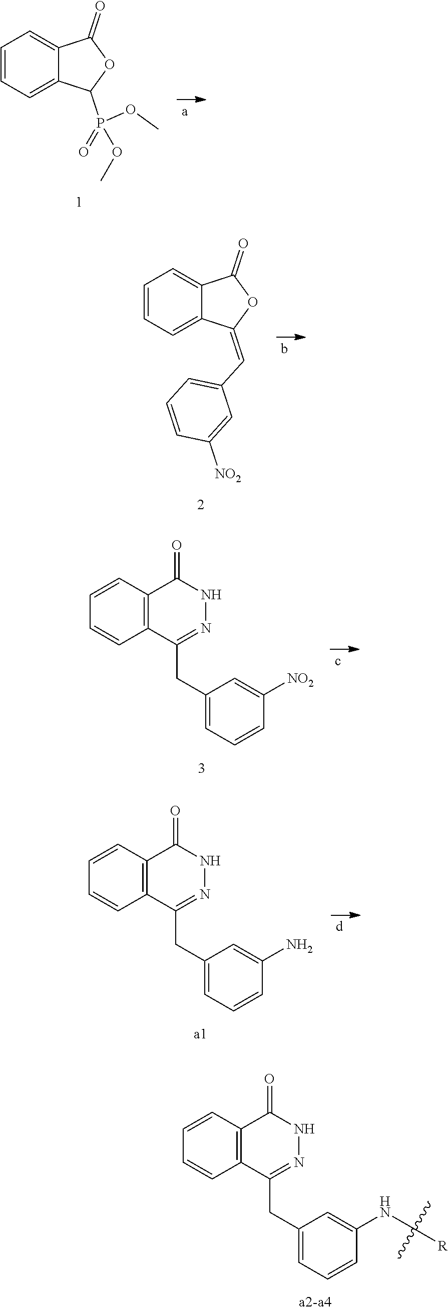

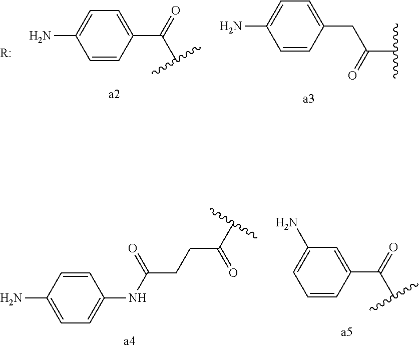

Synthesis of intermediates a2-a4: Compound a1 (1 mmol) was respectively dissolved with p-aminobenzoic acid (1 mmol), phenylamino-phenylacetic acid (1 mmol), 4-[(4-aminophenyl)amino]-4-oxobutyric acid (1 mmol), and m-aminobenzoic acid (1 in 50 mL of dichloromethane (DCM). 1-Ethyl-3-(3-mmol) dimethylaminopropyl) carbodiimide (EDCI) (1 mmol) and 4-dimethylaminopyridine (DMAP) (0.05 mmol) were added, and the mixture was reacted at room temperature overnight. The reaction was monitored by TLC and confirmed to be complete. After completion of the reaction, the reaction mixture was poured into saturated ammonium chloride solution and extracted with DCM. An organic phase was collected, dried over anhydrous Na₂SO₄, and concentrated under reduced pressure. A crude product was purified by column chromatography to yield the intermediates a2-a4.

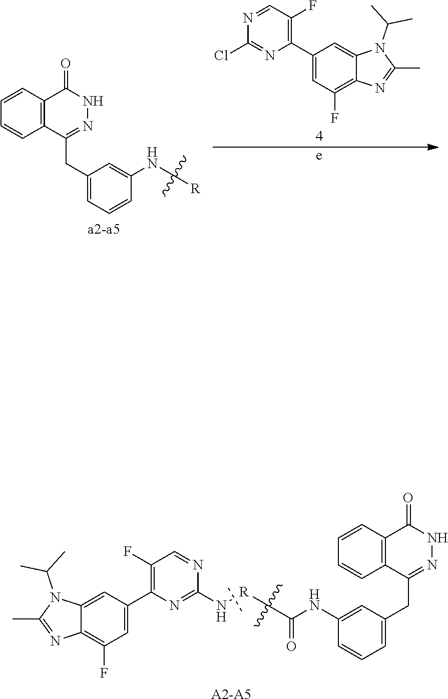

Synthesis of target compounds A2-A5: Compounds a2-a5 (0.5 mmol) and compound 4 (0.5 mmol) were dissolved in 50 mL of n-butanol. A catalytic amount of concentrated hydrochloric acid was added, and the mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the target compounds A2-A5.

Table 1 shows spectroscopic data of compounds A1-A5.

TABLE 1

| Serial | |
|---|---|
| Number | Spectroscopic data |
| A1 | ¹H NMR (300 MHz, DMSO-d6) δ 11.17 (s, 1H), 8.44 (d, J = 2.8 Hz, 1H), |
| | 8.34 (d, J = 3.8 Hz, 1H), 8.17 (s, 1H), 7.85-7.77 (m, 3H), 7.76-7.67 (m, |
| | 3H), 7.52 (s, 1H), 7.30-7.23 (m, 1H), 6.96 (s, 1H), 4.80-4.67 (m, 1H), |
| | 4.32 (s, 2H), 2.68 (s, 3H), 1.70 (s, 3H), 1.68 (s, 3H). |
| | ¹³C NMR (75 MHz, DMSO-d6) δ 160.49, 156.22, 153.55, 147.23, |
| | 146.87, 146.06, 145.98, 140.25, 138.58, 136.49, 136.37, 133.33, 131.17, |
| | 129.83, 129.15 (2C), 128.42, 128.37, 127.56, 126.77, 125.46, 122.39, |
| | 118.92, 117.14, 108.60, 108.15, 48.58, 38.93, 21.44 (2C), 15.07. |
| A2 | ¹H NMR (300 MHz, DMSO-d6) δ 12.65 (s, 1H), 10.15 (s, 1H), 10.04 |
| | (s, 1H), 8.70 (d, J = 3.8 Hz, 1H), 8.28 (d, J = 7.3 Hz, 1H), 8.24 (s, 1H), |
| | 7.96 (s, 1H), 7.91-7.89 (m, 2H), 7.87-7.84 (m, 2H), 7.81-7.77 (d, J = 6.8 |
| | Hz, 1H), 7.73-7.68 (m, 2H), 7.67 (s, 2H), 7.27 (d, J = 8.5 Hz, 1H), 7.10 |
| | (d, J = 7.6 Hz, 1H), 4.85 (q, J = 6.0 Hz, 1H), 4.31 (s, 2H), 2.65 (s, 3H), 1.64 |
| | (s, 3H), 1.62 (s, 3H). ¹³C NMR (75 MHz, DMSO-d6) δ 165.43, 162.77, |
| | 159.95, 156.38, 155.07, 151.18, 148.26, 145.56, 144.17, 140.10, 139.21, |
| | 136.84, 133.90, 131.91, 129.70, 129.22 (2C), 128.99, 128.42, 127.62 |
| | (2C), 127.06, 126.49, 126.16, 124.19, 121.19, 120.58, 119.01, 117.77 |
| | (2C), 109.40, 107.62, 48.60, 38.44, 21.44 (2C), 15.06. |
| A3 | White solid; yield: 46%; ¹H NMR (300 MHz, DMSO-d6) δ 12.62 (s, |
| | 1H), 9.93 (d, J = 8.9 Hz, 2H), 9.68 (s, 1H), 8.60 (d, J = 3.9 Hz, 1H), 8.26 |
| | (d, J = 6.3 Hz, 2H), 7.85 (d, J = 5.7 Hz, 2H), 7.83-7.78 (m, 1H), 7.71 (d, |
| | J = 8.9 Hz, 2H), 7.64 (s, 1H), 7.56 (d, J = 8.9 Hz, 2H), 7.51 (d, J = 8.7 Hz, |
| | 1H), 7.46 (s, 1H), 7.23 (t, J = 7.8 Hz, 1H), 7.03 (d, J = 7.4 Hz, 1H), 4.81 |
| | (p, 6.0 Hz, 1H), 4.27 (s, 2H), 2.63 (s, 3H), 2.62 (s, 4H), 1.63 (s, 3H), |
| | 1.60 (s, 3H). ¹³C NMR (75 MHz, DMSO-d6) δ 170.88, 170.35, 159.93, |
| | 156.84, 154.95, 154.37, 154.35, 152.05, 151.06, 148.71, 145.55, 140.04, |
| | 139.32, 136.69, 136.21, 134.12, 133.89, 133.54, 131.90, 129.65, 129.36, |
| | 128.38 (2C), 126.47, 126.11, 123.70, 119.67, 119.14 (4C), 117.56, |
| | 109.23, 107.55, 48.61, 31.57, 26.80, 21.38 (2C), 14.99. |
| A4 | White solid; yield: 37%; ¹H NMR (300 MHz, DMSO-d6) δ 12.65 (s, |
| | 1H), 10.20 (s, 1H), 9.96 (s, 1H), 8.67 (d, J = 3.9 Hz, 1H), 8.32-8.23 (m, |
| | 2H), 8.20 (d, J = 0.7 Hz, 1H), 8.00 (d, J = 8.0 Hz, 1H), 7.92 (d, J = 7.7 Hz, |
| | 1H), 7.90-7.84 (m, 1H), 7.84-7.78 (m, 1H), 7.69 (t, J = 8.5 Hz, 3H), 7.53 |
| | (d, J = 7.8 Hz, 1H), 7.44 (t, J = 7.9 Hz, 1H), 7.29 (t, J = 8.1 Hz, 1H), 7.13- |
| | 7.07 (m, 1H), 4.86-4.72 (m, 1H), 4.31 (s, 2H), 2.62 (s, 3H), 1.54 (d, |
| | J = 6.9 Hz, 6H). ¹³C NMR (75 MHz, DMSO-d6) δ 168.49, 162.15, |
| | 158.91, 157.16, 156.63, 154.55, 152.95, 151.20, 150.65, 147.76, 143.38, |
| | 142.16, 141.45, 139.11, 138.37, 136.11, 135.80, 134.11, 131.90, 131.45, |
| | 131.09, 130.62, 129.35, 128.70, 128.35, 126.61 (2C), 124.26, 123.19, |
| | 122.79, 121.17, 120.91, 111.37, 109.79, 50.72, 40.62, 23.58 (2C), 17.35. |
| A5 | ¹H NMR (300 MHz, DMSO-d6) δ 12.62 (s, 1H), 10.05 (s, 1H), 9.48 (s, |
| | 1H), 8.73 (s, 1H), 8.25 (d, J = 7.0 Hz, 1H), 7.88-7.81 (m, 2H), 7.78 (s, |
| | 1H), 7.75 (s, 1H), 7.51 (d, J = 8.5 Hz, 1H), 7.44 (s, 1H), 7.26-7.20 (m, |
| | 3H), 7.03 (d, J = 7.3 Hz, 1H), 6.57 (s, 1H), 4.78-4.66 (m, 1H), 4.25 (s, |
| | 2H), 3.52 (s, 2H), 3.36 (s, 6H), 3.05 (s, 6H), 2.46-2.36 (m, 2H), 1.99- |
| | 1.89 (m, 4H), 1.69-1.54 (m, 2H). ¹³C NMR (75 MHz, DMSO-d6) δ |
| | 169.84, 159.93, 156.78, 154.98, 154.40, 152.21, 151.11, 150.47, 148.80, |
| | 148.39, 145.52, 139.98, 139.55, 139.35, 136.81, 133.89, 133.56, 131.91, |
| | 129.59, 129.40 (2C), 128.39, 127.24, 126.48, 126.10, 123.97, 119.31, |
| | 119.19 (2C), 117.74, 109.26, 107.55, 48.58, 43.23, 38.30, 21.38 (2C), |
| | 15.03. |

### Example 2

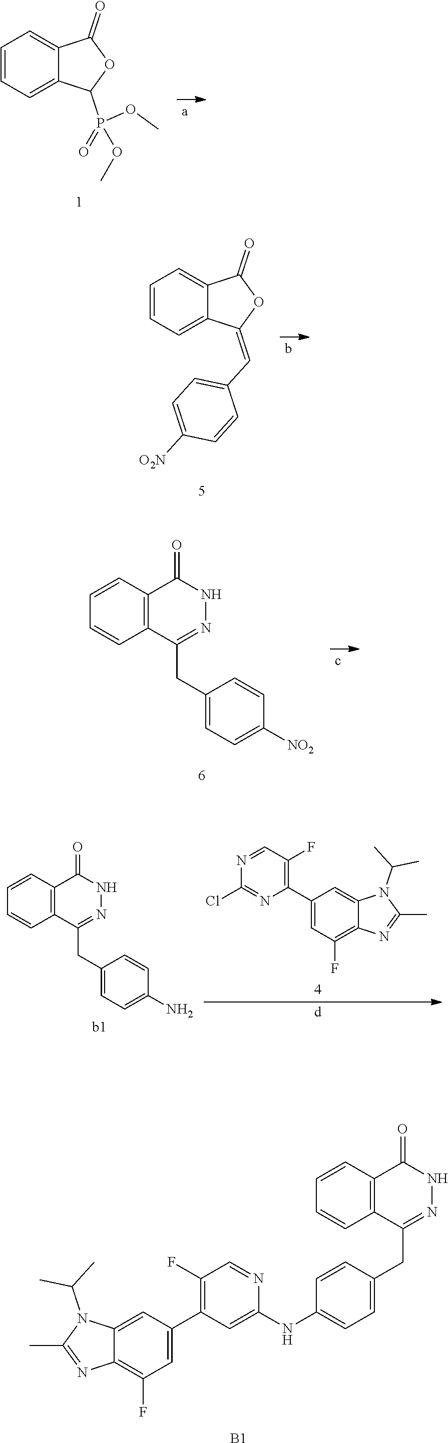

Synthesis of intermediate 5: Compound 1 (1 mmol) and m-nitrobenzaldehyde (1 mmol) were dissolved in 50 mL of tetrahydrofuran, and 5 mL of triethylamine was added. The mixture was reacted at room temperature overnight. The reaction was monitored by TLC and confirmed to be complete. After completion of the reaction, the reaction mixture was poured into a saturated sodium chloride solution and extracted with dichloromethane. An organic phase was collected, dried over anhydrous Na₂SO₄, and concentrated under reduced pressure. A crude product was purified by column chromatography to yield the intermediate 5.

Synthesis of intermediate 6: Compound 5 (1 mmol) and hydrazine hydrate (1 mmol) were dissolved in 50 mL of solvent mixture of ethanol and water (10:1). Sodium hydroxide (1 mmol) was added, and the mixture was reacted at 90 C overnight. The reaction was monitored by TLC and confirmed to be complete. After completion of the reaction, the reaction mixture was then poured into a saturated sodium chloride solution and extracted with dichloromethane. An organic phase was collected, dried over anhydrous Na₂SO₄, and concentrated under reduced pressure. A crude product was purified by column chromatography to yield the intermediate 6.

Synthesis of intermediate b1: Compound 6 (1 mmol) and zinc powder (3 mmol) were dissolved in 50 mL of solvent mixture of ethanol and water (10:1), and ammonium chloride (1 mmol) was added. The mixture was reacted at 90° C. overnight. The reaction was monitored by TLC and confirmed to be complete. After completion of the reaction, the reaction mixture was poured into a saturated sodium chloride solution and extracted with dichloromethane. An organic phase was collected, dried over anhydrous Na₂SO₄, and concentrated under reduced pressure. A crude product was purified by column chromatography to yield the intermediate b1.

Synthesis of target compound B1: Compound b1 (0.5 mmol) and compound 4 (0.5 mmol) were dissolved in n-butanol (50 mL). A catalytic amount of concentrated hydrochloric acid was added, and the mixture was reacted at 110° C. overnight. The reaction was monitored by TLC and confirmed to be complete. After completion of the reaction, the reaction mixture was poured into saturated sodium bicarbonate solution and extracted with dichloromethane. An organic phase was collected, dried over anhydrous Na₂SO₄, and concentrated under reduced pressure. A crude product was purified by column chromatography to yield the target compound B1.

Table 2 shows spectroscopic data of compound B1.

TABLE 2

| Serial | |
|---|---|
| Number | Spectroscopic data |
| B1 | ¹H NMR (300 MHz, CDCl₃) δ 11.40 (s, 1H), 8.44 (d, J = 5.5 Hz, 1H), 8.33 |
| | (d, J = 3.8 Hz, 1H), 8.18 (s, 1H), 7.76 (d, J = 11.8 Hz, 3H), 7.71 (s, 1H), |
| | 7.66 (d, J = 8.5 Hz, 2H), 7.34 (s, 1H), 7.26 (d, J = 7.9 Hz, 2H), 4.77-4.61 |
| | (m, 1H), 4.29 (s, 2H), 2.67 (s, 3H), 1.64 (s, 4H), 1.61 (s, 3H). ¹³C NMR |
| | (75 MHz, CDCl₃) δ 160.49, 156.24, 153.54, 150.97, 147.35, 146.99, |
| | 146.18, 138.62, 136.40, 136.27, 133.26, 131.47, 131.14, 129.82, 128.81 |
| | (2C), 128.40, 127.56, 127.42, 126.73, 125.49, 119.20 (2C), 108.69, |
| | 48.55, 38.27, 21.32 (2C), 14.98. |

### Example 3

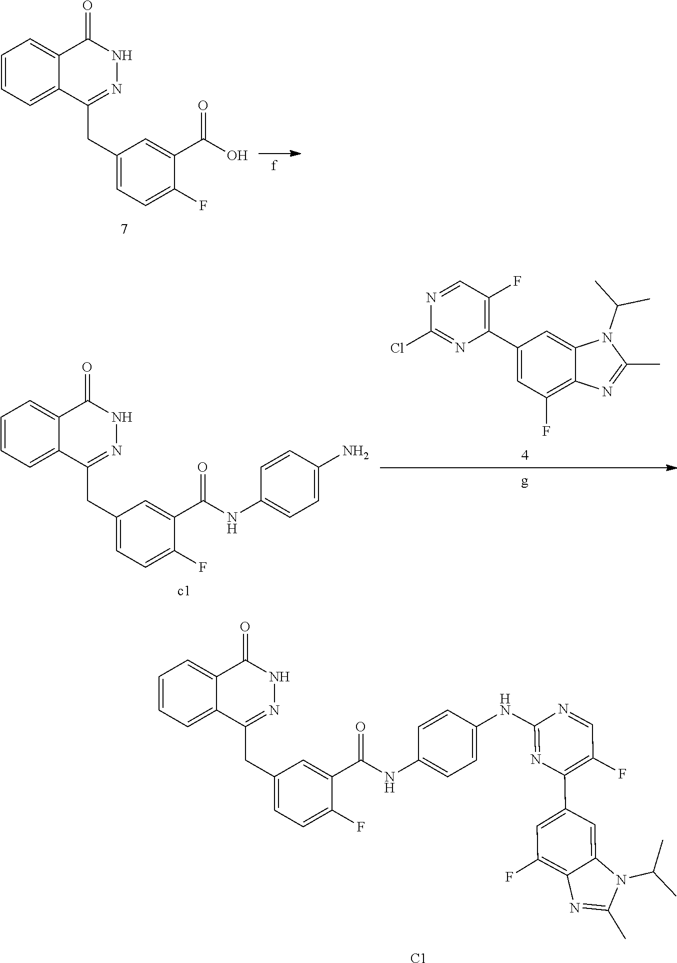 [SMILES: O=C(O)c1cc(Cc2n[nH]c(=O)c3ccccc23)ccc1F ; Nc1ccc(NC(=O)c2cc(Cc3n[nH]c(=O)c4ccccc34)ccc2F)cc1 ; Cc1nc2c(F)cc(-c3nc(Cl)ncc3F)cc2n1C(C)C ; Cc1nc2c(F)cc(-c3nc(Nc4ccc(NC(=O)c5cc(Cc6n[nH]c(=O)c7ccccc67)ccc5F)cc4)ncc3F)cc2n1C(C)C ; "C1"]

Synthesis of intermediate c1: Compound 7 (1 mmol) and p-phenylenediamine (1 mmol) were dissolved in 50 mL of DCM, and EDCI (1 mmol) and DMAP (0.05 mmol) were added. The mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediate c1.

Synthesis of target compound C1: Compound c1 (0.5 mmol) and compound 4 (0.5 mmol) were dissolved in 50 mL of n-butanol. A catalytic amount of concentrated hydrochloric acid was added, the mixture was reacted at 110° C. overnight. The reaction was monitored by TLC and confirmed to be complete. After completion of the reaction, the reaction mixture was poured into saturated sodium bicarbonate solution and extracted with dichloromethane. An organic phase was collected, dried over anhydrous Na₂SO₄, and concentrated under reduced pressure. A crude product was purified by column chromatography to yield the target compound C1.

### Example 4

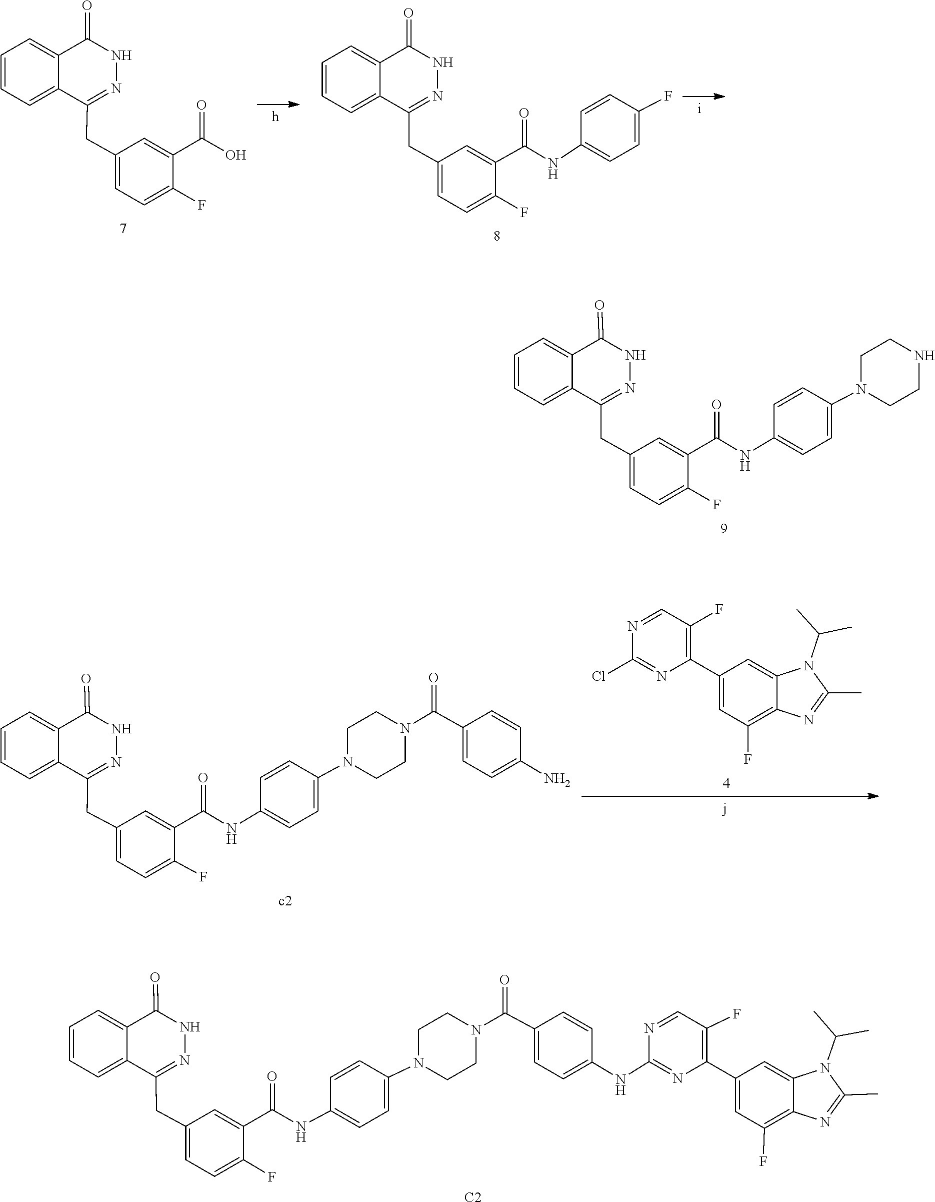

Synthesis of intermediate 8: Compound 7 (1 mmol) and 4-fluoroaniline (1 mmol) were dissolved in 50 mL of DCM, and EDCI (1.0 mmol) and DMAP (0.05 mmol) were added. The mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediate 8.

Synthesis of intermediate 9: Compound 8 (1 mmol) and piperazine (1 mmol) were dissolved in 50 mL of DMSO. K₂CO₃ (2 mmol) was added, and the mixture was refluxed overnight. The reaction was monitored by TLC and confirmed to be complete. After completion of the reaction, the reaction mixture was poured into a saturated sodium chloride solution and extracted with dichloromethane. An organic phase was collected, dried over anhydrous Na₂SO₄, and concentrated under reduced pressure. A crude product was purified by column chromatography to yield the intermediate 9.

Synthesis of intermediate c2: Compound 9 (1 mmol) and p-aminobenzoic acid (1 mmol) were dissolved in 50 mL of DCM. EDCI (1 mmol) and DMAP (0.05 mmol) were added, and the mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediate c2.

Synthesis of target compound C2: Compound c2 (0.5 mmol) and compound 4 (0.5 mmol) were dissolved in 50 mL of n-butanol. A catalytic amount of concentrated hydrochloric acid was added, and the mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the target compound C2.

### Example 5

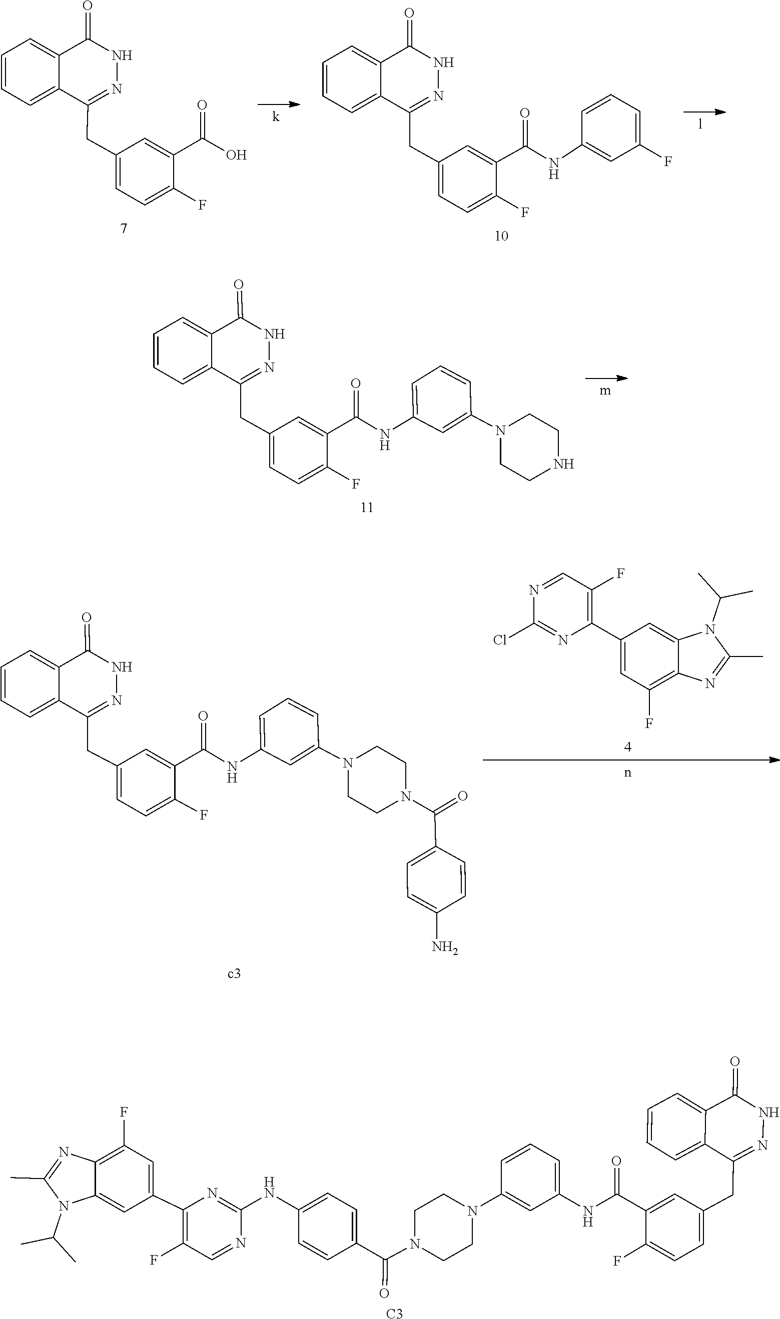

Synthesis of intermediate 10: Compound 7 (1 mmol) and m-fluoroaniline (1 mmol) were dissolved in 50 mL of DCM. EDCI (1 mmol) and DMAP (0.05 mmol) were added, and the mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediate 10.

Synthesis of intermediate 11: Compound 10 (1 mmol) and piperazine (1 mmol) were dissolved in 50 mL of DMSO. K₂CO₃ (2 mmol) was added, and the mixture was refluxed overnight. The reaction was monitored by TLC and confirmed to be complete. After completion of the reaction, the reaction mixture was poured into a saturated sodium chloride solution and extracted with dichloromethane. An organic phase was collected, dried over anhydrous Na₂SO₄, and concentrated under reduced pressure. A crude product was purified by column chromatography to yield the intermediate 11.

Synthesis of intermediate c3: Compound 11 (1 mmol) and p-aminobenzoic acid (1 mmol) were dissolved in 50 mL of DCM. EDCI (1 mmol) and DMAP (0.05 mmol) were added, and the mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediate c3.

Synthesis of target compound C3: Compound c3 (0.5 mmol) and Compound 4 (0.5 mmol) were dissolved in 50 mL of n-butanol. A catalytic amount of concentrated hydrochloric acid was added, and the mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the target compound C3.

### Example 6

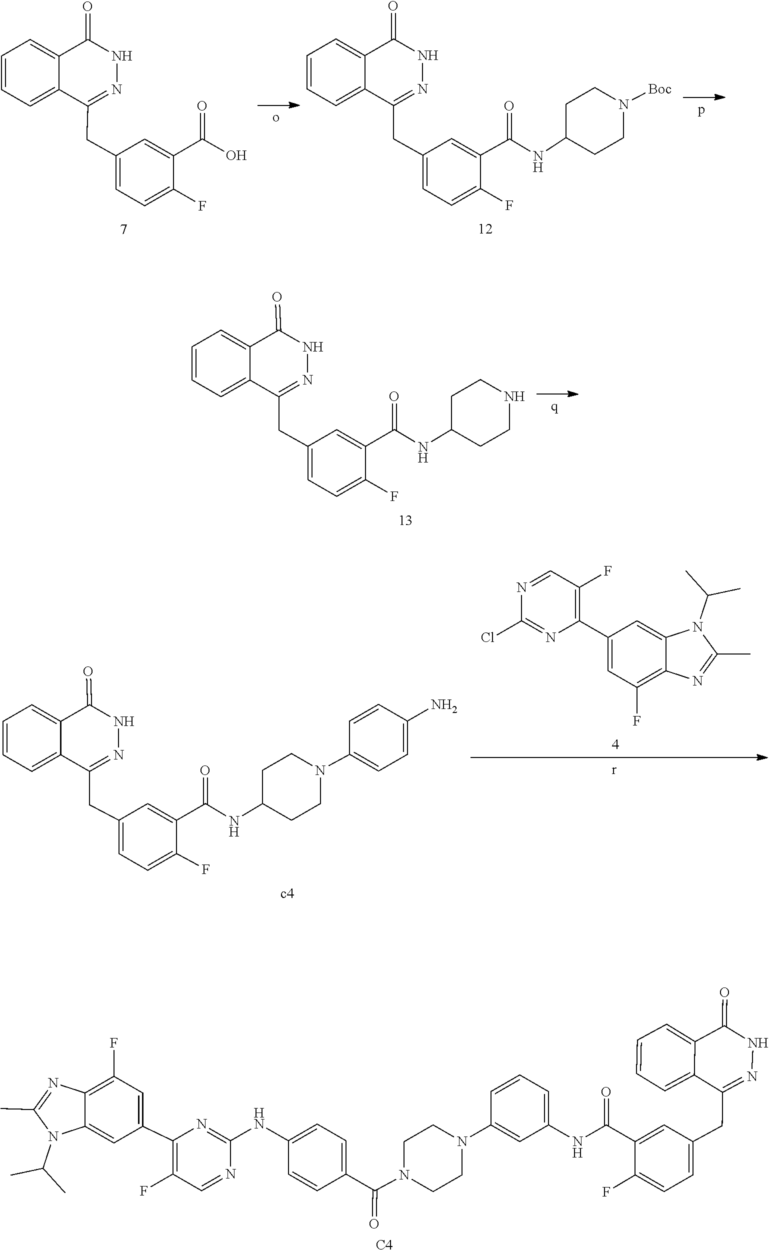

Synthesis of intermediate 12: Compound 7 (1 mmol) and 1-Boc-4-aminopiperidine (1 mmol) were dissolved in 50 mL of DCM. EDCI (1 mmol) and DMAP (0.05 mmol) were added, and the mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediate 12.

Synthesis of intermediate 13: Compound 12 (1 mmol) was dissolved in 50 mL of DCM, and trifluoroacetic acid (1 mmol) was added. The mixture was reacted at room temperature for 1 hour. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the intermediate 13.

Synthesis of intermediate c4: Compound 12 (1 mmol) and p-fluoroaniline (1 mmol) were dissolved in 50 mL of DMSO. Potassium carbonate (2 mmol) was added, and the mixture was refluxed overnight. The reaction was monitored by TLC and confirmed to be complete. After completion of the reaction, the reaction mixture was poured into a saturated sodium chloride solution and extracted with dichloromethane. An organic phase was collected, dried over anhydrous Na₂SO₄, and concentrated under reduced pressure. A crude product was purified by column chromatography to yield the intermediate c4.

Synthesis of target compound C4: Compound c4 (0.5 mmol) and compound 4 (0.5 mmol) were dissolved in 50 mL of n-butanol. A catalytic amount of concentrated hydrochloric acid was added, and the mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the target compound C4.

### Example 7

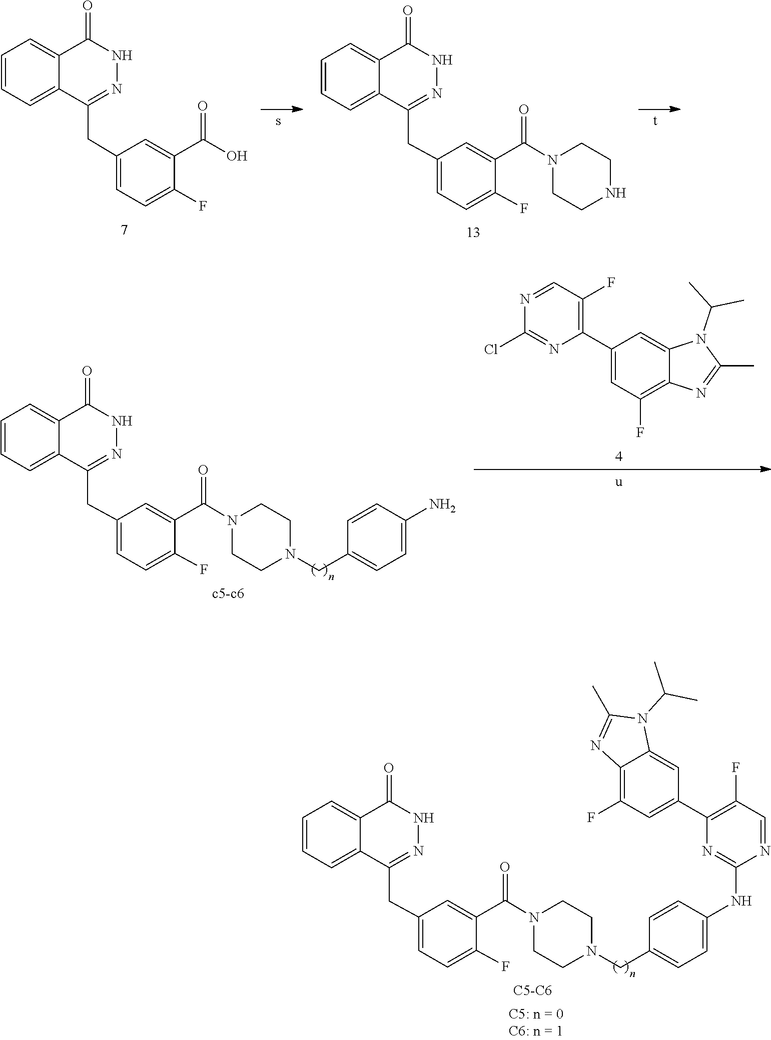 [SMILES: O=C(O)c1cc(Cc2n[nH]c(=O)c3ccccc23)ccc1F ; O=C(c1cc(Cc2n[nH]c(=O)c3ccccc23)ccc1F)N1CCNCC1 ; Nc1ccc(CN2CCN(C(=O)c3cc(Cc4n[nH]c(=O)c5ccccc45)ccc3F)CC2)cc1 ; Cc1nc2c(F)cc(-c3nc(Cl)ncc3F)cc2n1C(C)C ; Cc1nc2c(F)cc(-c3nc(Nc4ccc(CN5CCN(C(=O)c6cc(Cc7n[nH]c(=O)c8ccccc78)ccc6F)CC5)cc4)ncc3F)cc2n1C(C)C]

Synthesis of intermediate 13: Compound 7 (1 mmol) and piperazine (1 mmol) were dissolved in 50 mL of DCM. EDCI (1 mmol) and DMAP (0.05 mmol) were added, and the mixture was stirred at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediate 13.

Synthesis of intermediates c5 and c6: Intermediate 13 (1 mmol) and p-fluoroaniline (1 mmol) or p-aminobenzyl chloride (1 mmol) were dissolved in 50 mL of DMSO. Potassium carbonate (2 mmol) was added, and the mixture was refluxed overnight. The reaction was monitored by TLC and confirmed to be complete. After completion of the reaction, the reaction mixture was poured into a saturated sodium chloride solution and extracted with dichloromethane. An organic phase was collected, dried over anhydrous Na₂SO₄, and concentrated under reduced pressure. A crude product was purified by column chromatography to yield the intermediates c5 and c6.

Synthesis of target compounds C5 and C6: Compound c5 or c6 (0.5 mmol) and compound 4 (0.5 mmol) were dissolved in 50 mL of n-butanol. A catalytic amount of concentrated hydrochloric acid was added, and the mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the target compounds C5 and C6.

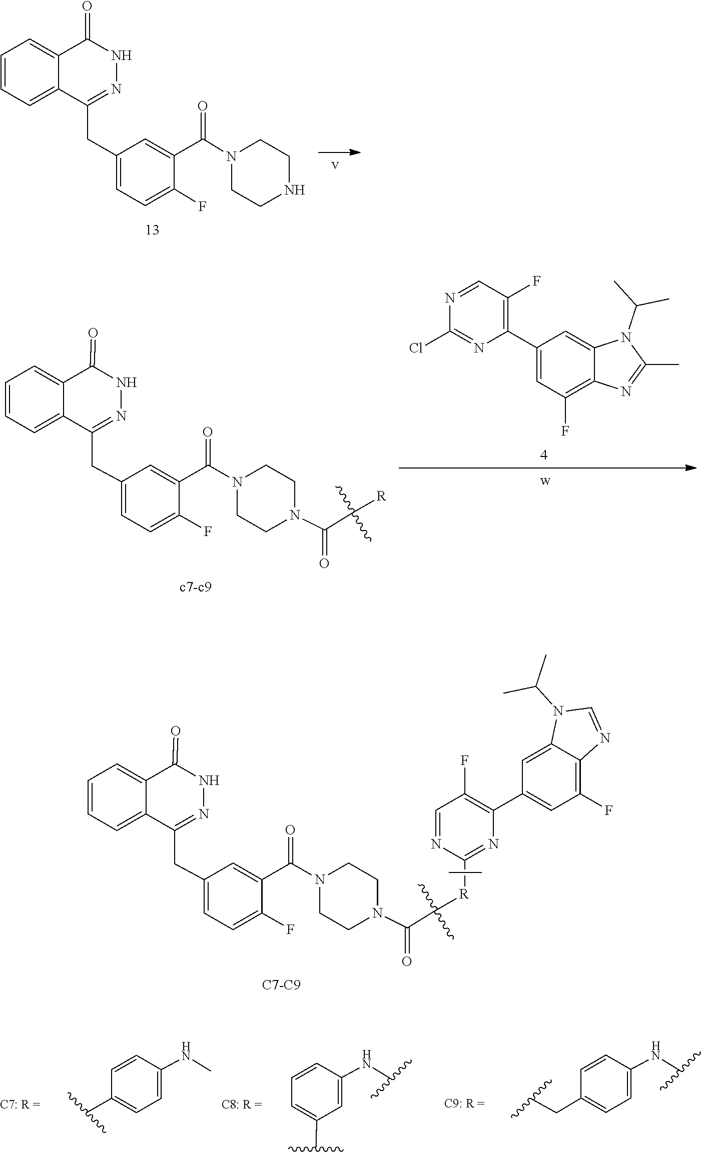

### Example 8

Synthesis of intermediates c7-c9: Intermediate 13 (1 mmol) was respectively dissolved with p-aminobenzoic acid (1 mmol), m-aminobenzoic acid (1 mmol), or p-amino-phenylacetic acid (1 mmol) in 50 mL of DCM. EDCI (1 mmol) and DMAP (0.05 mmol) were added, and the mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediates c7-c9.

Synthesis of target compounds C7-C9: Compounds c7-c9 (0.5 mmol) and compound 4 (0.5 mmol) were dissolved in 50 mL of n-butanol. A catalytic amount of concentrated hydrochloric acid was added, and the mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the target compounds C7-C9.

Table 3 shows spectroscopic data of compounds C1-C9.

TABLE 3

| Serial | |
|---|---|
| Number | Spectroscopic data |
| C1 | ¹H NMR (300 MHz, DMSO-d6) δ 12.63 (s, 1H), 10.33 (s, 1H), 9.78 (s, 1H), |
| | 8.62 (d, J = 4.0 Hz, 1H), 8.31-8.26 (m, 2H), 8.01 (t, J = 6.0 Hz, 1H), 7.91 (td, |
| | J = 7.7, 1.4 Hz, 1H), 7.84 (d, J = 7.4 Hz, 1H), 7.79 (d, J = 9.1 Hz, 2H), 7.72-7.68 |
| | (m, 2H), 7.67-7.63 (m, 2H), 7.55-7.48 (m, 1H), 7.35-7.20 (m, 2H), 4.89-4.80 |
| | (m, 1H), 4.37 (d, J = 5.7 Hz, 2H), 2.65 (s, 3H), 1.64 (d, J = 6.9 Hz, 6H). ¹³C |
| | NMR (75 MHz, DMSO-d6) δ 162.66, 159.89, 156.82, 156.46, 154.99, |
| | 152.12, 151.10, 148.78, 145.40, 137.08, 136.81, 134.92, 133.99, 133.68, |
| | 133.47, 132.02, 130.37, 129.55 (2C), 128.39, 127.32, 126.55, 126.01, |
| | 125.60, 125.39, 121.88, 120.53 (2C), 119.55 (2C), 116.76, 114.26, 48.60, |
| | 36.94, 21.41 (2C), 15.01. |
| C2 | ¹H NMR (300 MHz, DMSO-d6) δ 12.63 (s, 1H), 10.28 (s, 1H), 10.08 (s, 1H), |
| | 8.68 (d, J = 3.8 Hz, 1H), 8.27 (d, J = 7.9 Hz, 2H), 8.00 (d, J = 7.9 Hz, 1H), 7.92 |
| | (d, J = 8.8 Hz, 3H), 7.87-7.84 (m, 1H), 7.81 (d, J = 6.6 Hz, 1H), 7.67 (d, J = 12.2 |
| | Hz, 1H), 7.62 (dd, J = 6.8, 2.0 Hz, 1H), 7.54-7.48 (m, 1H), 7.44 (d, J = 8.7 Hz, |
| | 3H), 7.32-7.24 (m, 1H), 7.18 (t, J = 7.0 Hz, 2H), 4.89-4.80 (m, 1H), 4.37 (s, |
| | 2H), 3.68 (s, 4H), 3.18 (s, 4H), 2.62 (s, 3H), 1.64 (d, J = 6.9 Hz, 6H). ¹³C NMR |
| | (75 MHz, DMSO-d6) δ 169.62, 163.03, 159.87, 159.71, 156.50, 155.47, |
| | 155.02, 151.69, 150.76, 150.02, 148.25, 145.37, 142.53, 140.08, 136.82, |
| | 134.92, 133.99, 133.67, 132.98, 132.02 (2C), 130.33, 129.68, 129.53 (2C), |
| | 128.65 (2C), 128.39 (2C), 127.19, 127.01, 126.29, 125.42, 118.15 (2C), |
| | 116.72, 111.92, 109.25, 107.76, 107.41, 49.10, 48.59, 36.92, 26.80, 21.44 |
| | (2C), 15.00. |
| C3 | ¹H NMR (300 MHz, DMSO-d6) δ 12.63 (s, 1H), 10.23 (s, 1H), 10.07 (s, 1H), |
| | 8.67 (d, J = 3.8 Hz, 1H), 8.27 (dd, J = 7.8, 1.0 Hz, 1H), 8.23 (s, 1H), 8.16 (d, |
| | J = 7.6 Hz, 1H), 8.00 (d, J = 7.8 Hz, 1H), 7.95 (s, 1H), 7.93-7.88 (m, 2H), 7.87 |
| | (d, J = 3.0 Hz, 2H), 7.83 (d, J = 6.8 Hz, 1H), 7.67 (d, J = 12.3 Hz, 1H), 7.62 (dd, |
| | J = 6.8, 2.0 Hz, 1H), 7.54-7.47 (m, 1H), 7.38 (s, 1H), 7.31-7.22 (m, 1H), 7.15 |
| | (d, J = 6.8 Hz, 2H), 6.72 (d, J = 6.9 Hz, 1H), 4.88-4.79 (m, 1H), 4.36 (s, 2H), |
| | 4.07-3.93 (m, 1H), 3.72 (d, J = 11.9 Hz, 2H), 2.83 (d, J = 11.6 Hz, 2H), 2.64 (s, |
| | 3H), 1.88 (d, J = 10.3 Hz, 2H), 1.76-1.65 (m, 2H), 1.62 (d, J = 6.9 Hz, 6H). ¹³C |
| | NMR (75 MHz, DMSO-d6) δ 165.65, 162.98, 162.76, 159.88, 156.44, |
| | 155.03, 153.39, 151.53, 150.60, 149.08, 148.18, 145.38, 143.67, 140.13, |
| | 136.76, 134.95, 133.98, 133.70, 133.05, 132.01, 130.35, 129.66, 129.53 |
| | (2C), 128.52 (2C), 128.39, 127.70, 127.17, 127.00, 126.23, 125.42, 117.77 |
| | (2C), 116.69, 112.03, 110.48, 109.47, 107.47, 107.33, 48.58, 48.29, 47.19, |
| | 36.93, 36.23, 31.22, 21.43 (2C), 15.04. |
| C4 | ¹H NMR (300 MHz, DMSO-d6) δ 8.51 (s, 1H), 8.07 (s, 1H), 7.89 (s, 1H), |
| | 7.84 (s, 1H), 7.65 (d, J = 5.0 Hz, 2H), 7.59 (d, J = 5.0 Hz, 2H), 7.38 (d, J = 6.1 |
| | Hz, 2H), 7.00 (s, 2H), 6.76 (s, 2H), 6.62 (s, 1H), 5.21 (s, 1H), 4.77 (s, 1H), |
| | 3.96 (s, 2H), 3.58 (s, 1H), 3.42 (t, J = 11.0 Hz, 1H), 3.35 (d, J = 11.1 Hz, 1H), |
| | 3.18-3.13 (m, 1H), 3.09 (d, J = 11.0 Hz, 1H), 2.53 (s, 3H), 2.09-1.97 (s, 2H), |
| | 1.84-1.72 (m, 2H), 1.62 (d, J = 6.0 Hz, 6H). ¹³C NMR (75 MHz, DMSO-d6) |
| | δ 165.65, 162.98, 162.76, 159.88, 156.44, 155.03, 153.39, 151.53, 150.60, |
| | 149.08, 148.18, 145.38, 143.67, 140.13, 136.76, 134.95, 133.98, 133.70, |
| | 133.05, 132.01, 130.35, 129.66, 129.53, 128.52, 128.39, 127.70, 127.17, |
| | 127.00, 126.23, 121.11 (2C), 125.42 (2C), 117.77, 116.69, 112.03, 110.48, |
| | 109.47, 107.47, 107.33, 48.58, 48.29, 47.19, 36.93, 36.23, 31.22, 21.43 (2C), |
| | 15.04. |
| C5 | ¹H NMR (300 MHz, DMSO-d6) δ 12.62 (s, 1H), 9.55 (s, 1H), 8.54 (d, J = 3.9 |
| | Hz, 1H), 8.27 (d, J = 7.1 Hz, 1H), 8.21 (s, 1H), 7.97 (d, J = 7.7 Hz, 1H), 7.88 (t, |
| | J = 7.0 Hz, 1H), 7.81 (t, J = 7.4 Hz, 1H), 7.66-7.61 (m, 3H), 7.46-7.43 (m, 1H), |
| | 7.40 (d, J = 6.8 Hz, 1H), 7.24 (t, J = 8.9 Hz, 1H), 6.92 (d, J = 9.0 Hz, 2H), 4.85- |
| | 4.76 (m, 1H), 4.34 (s, 2H), 3.79 (s, 2H), 3.34 (s, 2H), 3.13 (s, 2H), 2.98 (s, |
| | 2H), 2.62 (s, 3H), 1.61 (s, 3H), 1.59 (s, 3H). ¹³C NMR (75 MHz, DMSO-d6) |
| | δ 164.28, 159.87, 158.50, 157.05, 155.26, 154.87, 153.17, 149.80, 148.08, |
| | 146.26, 145.34, 136.79, 135.32, 133.94, 133.56, 132.15, 131.99, 129.56, |
| | 129.39, 128.37, 127.45, 127.28, 126.54, 125.90, 124.20, 120.62 (2C), 117.13 |
| | (2C), 116.38, 109.18, 107.39, 49.83, 48.58, 46.96, 41.80, 36.91, 26.79, 21.42 |
| | (2C), 15.01. |
| C6 | ¹H NMR (300 MHz, DMSO-d6) δ 12.62 (s, 1H), 9.78 (s, 1H), 8.61 (d, J = 3.9 |
| | Hz, 1H), 8.28-8.23 (m, 2H), 7.95 (d, J = 7.5 Hz, 1H), 7.90-7.83 (m, 1H), 7.81 |
| | (dd, J = 7.5, 1.1 Hz, 1H), 7.76 (d, J = 8.5 Hz, 2H), 7.66 (d, J = 12.4 Hz, 1H), |
| | 7.45-7.37 (m, 1H), 7.32 (dd, J = 7.2, 3.6 Hz, 1H), 7.23 (d, J = 8.4 Hz, 3H), 4.88- |
| | 4.79 (m, 1H), 4.32 (s, 2H), 3.62 (s, 2H), 3.48-3.41 (m, 2H), 3.17 (s, 2H), 2.64 |
| | (s, 3H), 2.37 (d, J = 17.6 Hz, 2H), 2.27 (s, 2H), 1.62 (s, 3H), 1.60 (s, 3H). ¹³C |
| | NMR (75 MHz, DMSO-d6) δ 164.18, 159.84, 158.41, 156.78, 155.18, |
| | 154.96, 153.30, 149.78, 148.17, 145.33, 139.99, 136.73, 135.28, 133.91, |
| | 133.66, 131.97, 131.06, 129.64, 129.26, 128.36 (2C), 127.33, 127.16, |
| | 126.54, 125.91, 124.32, 119.04 (2C), 116.38, 109.24, 107.44, 61.91, 53.11, |
| | 48.57, 47.07, 41.86, 36.88, 26.79, 21.41 (2C), 15.02. |
| C7 | ¹H NMR (300 MHz, DMSO-d6) δ 12.61 (s, 1H), 10.07 (s, 1H), 8.66 (d, J = 3.6 |
| | Hz, 1H), 8.25 (d, J = 10.8 Hz, 2H), 7.97-7.87 (m, 4H), 7.81 (dd, J = 14.2, 6.7 |
| | Hz, 1H), 7.66 (d, J = 11.9 Hz, 1H), 7.48-7.36 (m, 4H), 7.24 (t, J = 9.0 Hz, 1H), |
| | 4.89-4.80 (m, 1H), 4.34 (s, 2H), 3.70 (s, 2H), 3.60 (s, 2H), 3.47 (s, 2H), 3.27 |
| | (s, 2H), 2.64 (s, 3H), 1.62 (d, J = 6.8 Hz, 6H). ¹³C NMR (75 MHz, DMSO-d6) |
| | δ 169.79, 164.54, 159.86, 158.49, 156.48, 155.25, 155.02, 154.43, 152.39, |
| | 151.13, 150.77, 149.04, 148.18, 145.28, 142.63, 136.85, 135.27, 133.91, |
| | 133.69, 132.25, 131.98, 129.54, 129.39, 128.70, 128.37, 128.23 (2C), |
| | 126.54, 125.91, 124.03, 118.10 (2C), 116.37, 48.58, 36.92, 26.80, 21.42 (2C), |
| | 15.05. |
| C8 | ¹H NMR (300 MHz, DMSO-d6) δ 12.62 (s, 1H), 9.97 (s, 1H), 8.67 (d, J = 3.7 |
| | Hz, 1H), 8.26 (d, J = 6.0 Hz, 1H), 8.19 (s, 1H), 7.94 (d, J = 8.3 Hz, 2H), 7.86 |
| | (s, 3H), 7.66 (d, J = 12.1 Hz, 1H), 7.43-7.35 (m, 2H), 7.24 (t, J = 8.8 Hz, 1H), |
| | 7.09-7.01 (m, 1H), 6.66-6.46 (m, 1H), 4.90-4.79 (m, 1H), 4.33 (s, 2H), 3.78- |
| | 3.57 (m, 4H), 3.51 (s, 2H), 3.36 (s, 2H), 3.26 (s, 2H), 2.64 (s, 3H), 1.62 (s, |
| | 3H), 1.60 (s, 3H). ¹³C NMR (75 MHz, DMSO-d6) δ 169.78, 164.55, 159.85, |
| | 158.48, 156.63, 155.23, 155.00, 154.47, 151.69, 150.75, 149.17, 148.10, |
| | 145.29, 141.17, 135.30, 134.94, 133.67, 131.97, 129.54, 129.37, 128.37, |
| | 126.54, 125.91, 123.97, 120.38, 119.97, 117.38, 116.44, 115.38, 114.37, |
| | 112.59, 109.32, 107.40, 48.56, 36.92, 26.80, 21.44 (2C), 15.11. |
| C9 | ¹H NMR (300 MHz, DMSO-d6) δ 12.62 (s, 1H), 9.78 (s, 1H), 8.62 (d, J = 3.7 |
| | Hz, 1H), 8.25 (d, J = 6.7 Hz, 2H), 7.95 (t, J = 6.7 Hz, 1H), 7.87-7.80 (m, 2H), |
| | 7.75 (dd, J = 8.2, 3.4 Hz, 2H), 7.66 (d, J = 12.1 Hz, 1H), 7.42 (s, 1H), 7.37 (d, |
| | J = 6.4 Hz, 1H), 7.27-7.12 (m, 3H), 4.84 (d, J = 3.5 Hz, 1H), 4.31 (d, J = 4.6 Hz, |
| | 2H), 3.73-3.68 (m, 2H), 3.58 (s, 4H), 3.44 (s, 2H), 3.15 (d, J = 7.6 Hz, 2H), |
| | 2.64 (s, 3H), 1.65-1.57 (m, 6H). ¹³C NMR (75 MHz, DMSO-d6) δ 169.83, |
| | 164.46, 159.85, 158.41, 156.81, 154.97, 153.20, 152.13, 148.79, 148.24, |
| | 145.29, 139.45, 136.87, 136.74, 135.28, 133.91, 133.69, 132.25, 131.99, |
| | 129.49, 128.96, 128.35 (2C), 127.33, 127.16, 126.52, 125.89, 123.95, 119.20 |
| | (2C), 116.32, 109.18, 107.45, 48.57, 46.94, 45.76, 41.99, 41.31, 36.90, 26.79, |
| | 21.41 (2C), 15.03. |

### Example 9

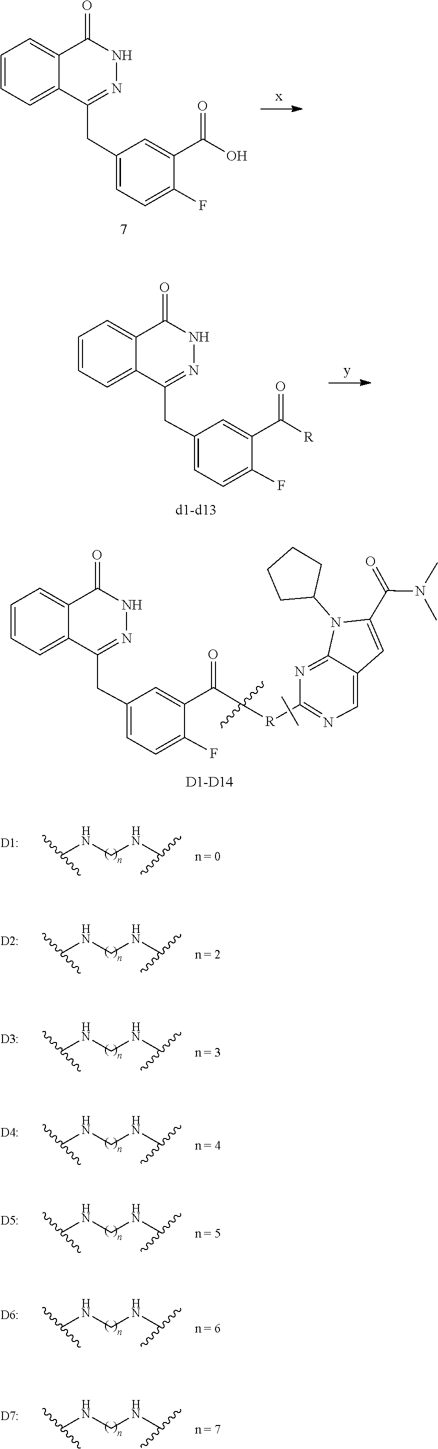

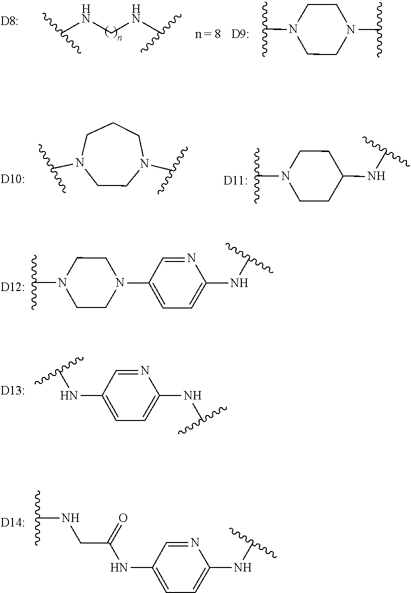

Synthesis of intermediates d1-d14: Compound 7 (1 mmol) was respectively dissolved with a selected series of amines (1 mmol) in 50 mL of DCM. EDCI (1 mmol) and DMAP (0.05 mmol) were added, and the mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediates d1-d14.

Synthesis of target compounds D1-D14: Compounds d1-d14 (1 mmol) and Compound 4 (1 mmol) were dissolved in 50 mL of n-butanol. A catalytic amount of concentrated hydrochloric acid was added, and the mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the target compounds D1-D14.

Table 4 shows spectroscopic data of compounds D1-D14.

TABLE 4

| Serial | |
|---|---|
| Number | Spectroscopic data |
| D1 | ¹H NMR (300 MHz, Chloroform-d) δ 9.08 (d, J = 8.4 Hz, 1H), 8.29 (s, 1H), |
| | 8.02 (d, J = 5.4 Hz, 1H), 7.90 (s, 1H), 7.62 (d, J = 5.0 Hz, 1H), 7.38 (s, 3H), |
| | 7.08 (s, 1H), 6.87-6.71 (m, 1H), 6.07 (s, 1H), 4.38-4.19 (m, 1H), 3.97 (s, 2H), |
| | 2.77 (s, 6H), 2.67 (s, 1H), 1.98 (d, J = 11.0 Hz, 2H), 1.53 (s, 2H), 1.33 (s, 2H), |
| | 1.03 (s, 2H). ¹³C NMR (75 MHz, Chloroform-d + DMSO-d₆) δ 168.60(2C), |
| | 165.25(2C), 156.94, 156.57, 139.37, 138.08, 136.94, 136.02, 134.20, 131.63, |
| | 129.72, 118.05, 105.42, 62.40, 42.53(2C), 40.18, 35.12(2C), 29.34(2C). |
| D2 | ¹H NMR (300 MHz, Chloroform-d) δ 10.79 (s, 1H), 8.55 (s, 1H), 8.43 (d, |
| | J = 8.3 Hz, 1H), 8.07 (d, J = 7.4 Hz, 1H), 7.74 (s, 3H), 7.31 (s, 2H), 6.97 (dd, |
| | J = 11.4, 8.4 Hz, 1H), 6.37 (s, 1H), 4.88-4.72 (m, 1H), 4.29 (s, 2H), 3.81 (s, |
| | 4H), 3.15 (s, 6H), 2.44 (s, 1H), 2.06 (s, 4H), 1.77 (s, 2H), 1.66 (s, 2H). ¹³C |
| | NMR (75 MHz, Chloroform-d) δ 164.41 (2C), 163.02, 160.68, 158.84, |
| | 153.30, 151.67, 145.62, 134.26, 133.61, 133.03, 132.00, 131.48, 130.58, |
| | 129.58, 128.37, 127.12, 125.09, 116.77, 116.26, 110.79, 101.74, 57.33, |
| | 41.75, 37.86, 30.57 (2C), 29.47, 26.81 (2C), 25.28(2C). |
| D3 | ¹H NMR (300 MHz, Chloroform-d) δ 10.61 (s, 1H), 8.53 (s, 1H), 8.45 (d, |
| | J = 6.8 Hz, 1H), 8.11 (d, J = 7.0 Hz, 1H), 7.76 (s, 3H), 7.34 (s, 2H), 7.05 (dd, |
| | J = 11.5, 8.4 Hz, 1H), 6.37 (s, 1H), 4.77 (t, J = 8.7 Hz, 1H), 4.38 (s, 2H), 3.70- |
| | 3.55 (m, 4H), 3.15 (s, 6H), 2.45 (s, 2H), 1.97 (s, 6H), 1.60 (s, 2H), 1.28 (s, |
| | 1H). ¹³C NMR (75 MHz, Chloroform-d) δ 164.30, 163.36, 161.37 (d, |
| | J = 245.25 Hz), 160.68, 158.69, 153.19, 151.20, 145.63, 134.25 (d, J = 3.3 Hz), |
| | 133.58, 132.96 (d, J = 9.0 Hz), 131.90, 131.43, 130.96, 129.55, 128.32, |
| | 127.05, 125.09, 121.43 (d, J = 11.9 Hz), 116.42 (d, J = 25.1 Hz), 111.00, |
| | 101.23, 57.31, 39.08 (2C), 37.85 (2C), 37.62, 30.58 (2C), 29.47, 25.18 (2C) |
| D4 | ¹H NMR (300 MHz, Chloroform-d) δ 10.87 (s, 1H), 8.52 (s, 1H), 8.47 (s, |
| | 1H), 8.08 (d, J = 9.9 Hz, 1H), 7.75 (s, 3H), 7.33 (s, 1H), 7.02 (dd, J = 11.6, 8.4 |
| | Hz, 1H), 6.84 (s, 1H), 6.36 (s, 1H), 4.76 (t, J = 8.5 Hz, 1H), 4.39 (s, 2H), 3.53 |
| | (d, J = 15.3 Hz, 2H), 3.16 (s, 6H), 2.43 (d, J = 20.2 Hz, 2H), 2.02 (s, 2H), 1.82 |
| | (s, 2H), 1.76 (s, 4H), 1.64 (s, 2H), 1.28 (s, 1H), 0.94-0.76 (m, 2H). ¹³C NMR |
| | (75 MHz, Chloroform-d) δ 164.40 (2C), 163.14, 160.66, 158.84, 153.09, |
| | 151.78, 145.56, 134.26, 133.55, 133.12, 131.90, 131.42, 130.53, 129.55, |
| | 128.34, 127.05, 125.05, 121.17, 116.58, 116.24, 110.82, 101.26, 57.31, 41.43 |
| | (2C), 39.93 (2C), 37.83, 30.56 (2C), 27.10 (2C), 25.25 (2C) |
| D5 | ¹H NMR (300 MHz, Chloroform-d) δ 11.45 (s, 1H), 8.53 (s, 1H), 8.48-8.42 |
| | (m, 1H), 8.07 (dd, J = 7.3, 2.3 Hz, 1H), 7.77-7.69 (m, 3H), 7.35-7.28 (m, 1H), |
| | 7.00 (dd, J = 11.5, 8.5 Hz, 1H), 6.84-6.72 (m, 1H), 6.34 (s, 1H), 4.75 (p, J = 8.6 |
| | Hz, 1H), 4.30 (s, 2H), 3.52-3.42 (m, 4H), 3.14 (s, 6H), 2.46 (dd, J = 12.1, 8.5 |
| | Hz, 2H), 2.01 (s, 4H), 1.67 (dt, J = 13.4, 7.2 Hz, 6H), 1.51 (dd, J = 15.0, 8.6 |
| | Hz, 2H), 1.26 (s, 1H). ¹³C NMR (75 MHz, Chloroform-d) δ 164.40, 163.14, |
| | 160.86, 158.89, 157.89 (d, J = 245.25 Hz), 153.15, 151.72, 145.54, 134.34 |
| | (d, J = 3.3 Hz), 133.51, 132.94 (d, J = 9.3 Hz), 131.83, 131.39, 130.50, 129.54, |
| | 128.34, 127.03, 125.08, 121.33 (d, J = 12.0 Hz), 116.39 (d, J = 25.1 Hz), |
| | 110.74, 101.30, 60.40, 57.30 (2C), 41.61, 40.01, 37.86, 30.56, 29.31 (2C), |
| | 29.19 (2C), 25.26 (2C), 24.45. |
| D6 | ¹H NMR (300 MHz, Chloroform-d) δ 10.60 (s, 1H), 8.52 (s, 1H), 8.47 (s, |
| | 1H), 8.10 (d, J = 6.9 Hz, 1H), 7.76 (s, 3H), 7.34 (s, 1H), 7.05 (t, J = 10.4 Hz, |
| | 1H), 6.77 (s, 1H), 6.36 (s, 1H), 4.77 (s, 1H), 4.32 (s, 2H), 3.47 (s, 4H), 3.16 |
| | (s, 6H), 2.49 (s, 2H), 2.04 (s, 2H), 1.83 (s, 2H), 1.67 (d, J = 11.8 Hz, 4H), 1.46 |
| | (s, 2H), 1.29 (s, 1H), 0.95-0.81 (m, 4H). ¹³C NMR (126 MHz, Chloroform- |
| | d + DMSO-d₆) δ 168.72, 168.60, 164.81, 163.69, 162.49, 157.67, 156.54, |
| | 149.65, 139.17, 138.20, 137.58, 136.11, 135.71, 135.38, 134.30, 133.24, |
| | 131.28, 130.12, 128.10, 121.18, 121.00, 105.83, 61.67, 46.05 (2C), 42.18 |
| | (3C), 35.26 (2C), 34.29, 34.08, 33.95, 33.89, 29.92 (2C). |
| D7 | ¹H NMR (300 MHz, Chloroform-d) δ 11.47 (s, 1H), 8.45 (s, 1H), 8.41-8.35 |
| | (m, 1H), 8.01 (dd, J = 7.4, 2.5 Hz, 1H), 7.67-7.63 (m, 3H), 7.25 (ddd, J = 8.4, |
| | 4.8, 2.5 Hz, 1H), 6.94 (dd, J = 11.6, 8.5 Hz, 1H), 6.67 (dt, J = 12.0, 5.6 Hz, 1H), |
| | 6.27 (s, 1H), 4.68 (p, J = 8.7 Hz, 1H), 4.23 (s, 2H), 3.36 (p, J = 6.6 Hz, 4H), |
| | 3.06 (s, 6H), 2.39 (ddd, J = 14.7, 9.6, 6.3 Hz, 2H), 2.01-1.89 (m, 4H), 1.61- |
| | 1.49 (m, 6H), 1.31 (d, J = 3.5 Hz, 6H), 1.18 (s, 1H). ¹³C NMR (75 MHz, |
| | Chloroform-d) δ 164.43, 163.08 (d, J = 3.2 Hz), 160.46, 158.95, 157.86(d, |
| | J = 245.25 Hz), 153.18, 151.31, 145.54, 134.38 (d, J = 3.2 Hz), 133.52, 132.97 |
| | (d, J = 9.3 Hz), 131.87, 131.39, 130.43, 129.55, 127.99, 127.05, 125.10, |
| | 121.29 (d, J = 12.3 Hz), 116.42 (d, J = 25.4 Hz), 110.69, 101.31, 57.30, 41.81, |
| | 40.05, 37.88, 30.55(2C), 29.47, 29.39, 29.12(2C), 27.00, 26.91, 25.28(3C). |
| D8 | ¹H NMR (300 MHz, Chloroform-d) δ 10.35 (s, 1H), 8.49 (s, 1H), 8.44 (d, |
| | J = 8.4 Hz, 1H), 8.08 (d, J = 8.6 Hz, 1H), 7.82-7.67 (m, 3H), 7.36 (s, 1H), 7.03 |
| | (dd, J = 11.5, 8.4 Hz, 1H), 6.73 (s, 1H), 6.33 (s, 1H), 4.81-4.66 (m, 1H), 4.29 |
| | (s, 2H), 3.42 (dt, J = 13.4, 6.6 Hz, 4H), 3.13 (s, 6H), 2.46 (s, 2H), 2.01 (s, 4H), |
| | 1.62 (s, 6H), 1.35 (s, 8H), 1.24 (s, 1H). ¹³C NMR (75 MHz, Chloroform-d) δ |
| | 164.41, 163.04, 160.87, 158.87, 157.86 (d, J = 244.5 Hz), 153.20, 151.60, |
| | 145.55, 134.38 (d, J = 3.3 Hz), 133.52, 132.97 (d, J = 9.2 Hz), 131.91, 131.39, |
| | 130.50, 129.55, 128.35, 127.04, 125.10, 121.28 (d, J = 12.0 Hz), 116.41 (d, |
| | J = 25.2 Hz), 110.66, 101.32, 57.32, 41.84, 40.08, 37.88, 30.55(2C), 29.50, |
| | 29.40, 29.34(2C), 29.22, 27.01, 26.87, 25.27(3C). |
| D9 | ¹H NMR (300 MHz, Chloroform-d) δ 10.53 (s, 1H), 8.73 (s, 1H), 8.49 (d, |
| | J = 8.7 Hz, 1H), 8.40 (d, J = 9.0 Hz, 1H), 8.07 (d, J = 26.4 Hz, 1H), 7.79 (s, 1H), |
| | 7.43-7.33 (m, 2H), 7.07 (t, J = 8.8 Hz, 1H), 6.46 (s, 1H), 4.82 (t, J = 9.0 Hz, |
| | 1H), 4.33 (s, 2H), 3.99 (s, 2H), 3.51 (s, 2H), 3.18 (s, 6H), 3.09 (s, 2H), 2.60 |
| | (s, 2H), 2.08 (s, 2H), 1.70 (s, 4H), 1.29 (s, 2H). ¹³C NMR (75 MHz, |
| | Chloroform-d) δ 165.16, 164.29, 160.85, 158.29, 155.43, 152.86, 151.43, |
| | 145.58, 134.38, 133.64, 131.56, 131.44, 129.58, 129.25, 128.33, 127.16, |
| | 125.10, 124.12, 116.35, 116.05, 111.30, 100.87, 57.36, 46.94(2C), 44.95, |
| | 44.43, 42.00(2C), 37.78, 30.47(2C), 25.21(2C). |
| D10 | ¹H NMR (300 MHz, Chloroform-d) δ 11.56 (d, J = 11.3 Hz, 1H), 8.60-8.42 |
| | (m, 2H), 7.76-7.64 (m, 3H), 7.21 (dd, J = 5.4, 2.9 Hz, 1H), 6.99 (dt, J = 23.9, |
| | 8.7 Hz, 1H), 6.84 (dd, J = 6.2, 2.3 Hz, 1H), 6.37 (d, J = 5.7 Hz, 1H), 4.73 (td, |
| | J = 8.7, 6.4 Hz, 1H), 4.26 (s, 1H), 4.08 (d, J = 14.9 Hz, 3H), 3.93 (d, J = 5.0 Hz, |
| | 1H), 3.86 (t, J = 6.0 Hz, 1H), 3.56 (s, 1H), 3.23 (t, J = 6.3 Hz, 1H), 3.15 (s, 6H), |
| | 2.43 (s, 1H), 1.98 (s, 6H), 1.56 (s, 2H), 1.26 (s, 1H), 0.90-0.82 (m, 2H). ¹³C |
| | NMR (75 MHz, Chloroform-d) δ 166.42, 164.39, 160.78, 155.31(d, J = 228 |
| | Hz), 157.29, 153.09, 151.74, 145.46, 134.09 (d, J = 3.2 Hz), 133.53, 131.40, |
| | 130.83, 129.57, 128.57 (d, J = 4.0 Hz), 128.29, 127.05, 125.12, 124.81, |
| | 116.02, 115.88 (d, J = 21.9 Hz), 110.66, 101.12, 57.30, 48.67, 48.20, 47.99, |
| | 47.89, 46.15, 45.01(2C), 37.83, 30.52(2C), 25.32(2C). |
| D11 | ¹H NMR (300 MHz, DMSO-d6) δ 12.61 (s, 1H), 8.56 (s, 1H), 8.26 (dd, J = 7.8, |
| | 1.5 Hz, 1H), 8.02-7.96 (m, 1H), 7.93-7.77 (m, 2H), 7.46-7.33 (m, 2H), 7.23 |
| | (t, J = 9.0 Hz, 1H), 6.99 (s, 1H), 6.47 (s, 1H), 4.69 (t, J = 8.5 Hz, 1H), 4.41 (d, |
| | J = 13.3 Hz, 1H), 4.34 (s, 2H), 3.95 (s, 1H), 3.41 (d, J = 13.7 Hz, 1H), 3.35 (s, |
| | 6H), 2.51 (q, J = 1.8 Hz, 4H), 2.32 (s, 2H), 1.92 (d, J = 9.4 Hz, 4H), 1.59 (d, |
| | J = 5.2 Hz, 2H), 1.23 (d, J = 1.8 Hz, 2H). ¹³C NMR (75 MHz, DMSO-d6) δ |
| | 164.19, 163.60, 159.86 (2C), 158.46, 152.65, 145.40, 135.78, 135.32, |
| | 133.96, 132.03, 130.74, 129.55, 128.38, 126.56, 125.96, 124.81, 124.53, |
| | 116.50, 110.87, 101.16, 56.78 (2C), 48.41 (2C), 42.91, 36.92 (2C), 31.56, |
| | 30.66 (2C), 29.39, 25.33 (2C). |
| D12 | ¹H NMR (300 MHz, Chloroform-d) δ 10.46 (s, 1H), 8.66 (s, 1H), 8.49-8.45 |
| | (m, 1H), 8.19 (dd, J = 7.5, 2.4 Hz, 1H), 7.76 (dd, J = 3.6, 2.1 Hz, 3H), 7.73 (s, |
| | 2H), 7.62 (d, J = 8.8 Hz, 2H), 7.49 (s, 1H), 7.44-7.36 (m, 1H), 7.12 (dd, J = 11.8, |
| | 8.5 Hz, 1H), 6.43 (s, 1H), 4.80 (q, J = 8.9 Hz, 1H), 4.35 (s, 2H), 3.16 (s, 6H), |
| | 2.59 (s, 2H), 2.06 (s, 4H), 1.70 (s, 2H), 1.26 (s, 1H). ¹³C NMR (75 MHz, |
| | Chloroform-d) δ 164.60, 164.11, 160.95, 160.72, 155.25, 152.13, 151.36, |
| | 145.64, 137.33, 136.97, 134.59, 133.68, 132.11, 132.10, 131.68, 131.57, |
| | 129.56, 128.34, 127.15, 125.07, 121.27(3C), 119.10 (2C), 116.81, 112.21, |
| | 101.12, 57.99, 37.86 (2C), 37.59, 30.16 (2C), 24.67 (2C). |
| D13 | ¹H NMR (300 MHz, DMSO-d₆) δ 12.61 (s, 1H), 9.95 (s, 1H), 9.47 (s, 1H), |
| | 8.73 (s, 1H), 8.46 (d, J = 5.0 Hz, 1H), 8.27 (d, J = 7.7 Hz, 1H), 7.99 (d, J = 8.1 |
| | Hz, 1H), 7.87 (dtd, J = 20.4, 7.1, 1.4 Hz, 2H), 7.76 (d, J = 8.9 Hz, 2H), 7.71 |
| | (dd, J = 7.1, 2.4 Hz, 1H), 7.51 (d, J = 8.9 Hz, 3H), 7.26 (dd, J = 10.7, 8.5 Hz, |
| | 1H), 6.58 (s, 1H), 4.72 (p, J = 8.6 Hz, 1H), 4.36 (s, 2H), 4.06 (d, J = 5.7 Hz, |
| | 2H), 3.06 (s, 6H), 2.50 (s, 2H), 1.97 (s, 4H), 1.67 (s, 2H). ¹³C NMR (75 MHz, |
| | Chloroform-d + DMSO-d₆) δ 171.64, 168.51, 168.32, 164.85, 162.30, 160.72, |
| | 156.74, 156.64, 149.63, 141.90 (2C), 139.33, 139.29, 138.20, 137.17, |
| | 136.19, 136.09, 134.30, 133.25, 131.30, 130.10, 124.84 (2C), 123.58(2C), |
| | 121.37, 121.13, 116.49, 105.78, 62.29, 48.57, 42.24 (3C), 34.77 (2C), 29.39 |
| | (2C) |
| D14 | ¹H NMR (300 MHz, DMSO-d₆) δ 12.61 (s, 1H), 9.95 (s, 1H), 9.47 (s, 1H), |
| | 8.73 (s, 1H), 8.46 (d, J = 5.0 Hz, 1H), 8.27 (d, J = 7.7 Hz, 1H), 7.99 (d, J = 8.1 |
| | Hz, 1H), 7.87 (dtd, J = 20.4, 7.1, 1.4 Hz, 2H), 7.76 (d, J = 8.9 Hz, 2H), 7.71 |
| | (dd, J = 7.1, 2.4 Hz, 1H), 7.51 (d, J = 8.9 Hz, 3H), 7.26 (dd, J = 10.7, 8.5 Hz, |
| | 1H), 6.58 (s, 1H), 4.72 (p, J = 8.6 Hz, 1H), 4.36 (s, 2H), 4.06 (d, J = 5.7 Hz, |
| | 2H), 3.06 (s, 6H), 2.50 (s, 2H), 1.97 (s, 4H), 1.67 (s, 2H). ¹³C NMR (75 MHz, |
| | Chloroform-d + DMSO-d₆) δ 171.64, 168.51, 168.32, 164.85, 162.30, 160.72, |
| | 156.74, 156.64, 149.63, 141.90 (2C), 139.33, 139.29, 138.20, 137.17, |
| | 136.19, 136.09, 134.30, 133.25, 131.30, 130.10, 124.84 (2C), 123.58 (2C), |
| | 121.37, 121.13, 116.49, 105.78, 62.29, 48.57, 42.24 (3C), 34.77 (2C), 29.39 |
| | (2C). |

### Example 10

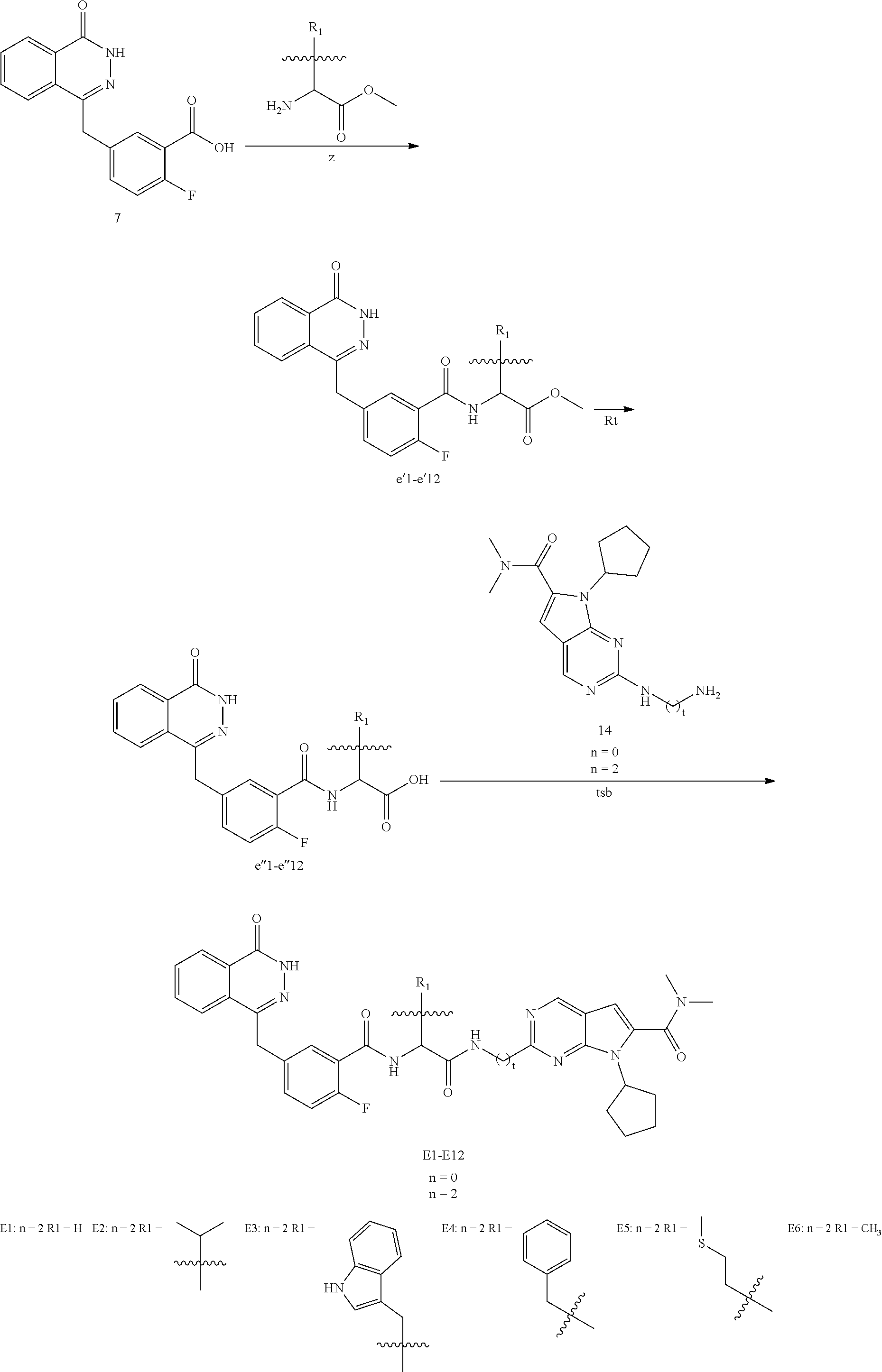

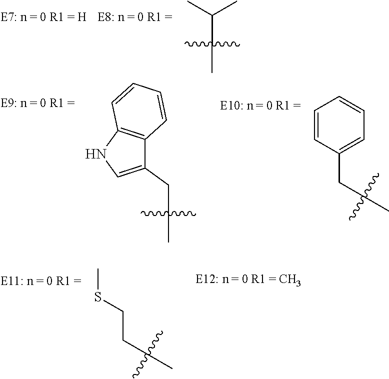

Synthesis of intermediates e′1-e′12: Compound 7 (1 mmol) and a selected series of amino acid methyl esters (1 mmol) were dissolved in 50 mL of DCM. EDCI (1 mmol) and DMAP (0.05 mmol) were added, and the reaction mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the intermediates e′1-e′12.

Synthesis of intermediates e″1-e″12: Compounds e′1-e′12 (1 mmol) were dissolved in 50 mL of ethanol and NaOH (1 mmol) was added. The reaction mixture was reacted at room temperature overnight. The reaction was monitored by TLC and confirmed to be complete. After completion of the reaction, the reaction mixture was poured into 10% hydrochloric acid solution and extracted with dichloromethane. An organic phase was collected, dried over anhydrous Na₂SO₄, and concentrated under reduced pressure. A crude product was purified by column chromatography to yield the intermediates e″1-e″ 12.

Synthesis of target compounds E1-E12: Compounds e″1-e″12 (1 mmol) and compound 14 (1 mmol) were dissolved in 50 mL of n-butanol and a catalytic amount of concentrated hydrochloric acid was added. The reaction mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the target compounds E1-E12.

Table 5 shows spectroscopic data of compounds E1-E12.

TABLE 5

| Serial | |
|---|---|
| Number | Spectroscopic data |
| E1 | ¹H NMR (300 MHz, Chloroform-d) δ 10.74 (s, 1H), 8.51 (s, 1H), 8.49-8.39 |
| | (m, 1H), 8.04 (d, J = 6.9 Hz, 1H), 7.76 (s, 3H), 7.35 (s, 1H), 7.16 (s, 1H), 7.10- |
| | 7.00 (m, 1H), 6.34 (s, 1H), 5.66 (s, 1H), 4.85-4.72 (m, 1H), 4.28 (s, 2H), 4.15 |
| | (s, 2H), 3.60 (s, 4H), 3.16 (s, 6H), 2.43 (s, 1H), 2.00 (s, 2H), 1.66 (s, 4H), |
| | 1.28 (s, 2H). ¹³C NMR (75 MHz, Chloroform-d) δ 168.97, 164.20, 163.48, |
| | 160.58, 158.64, 158.15, 152.91, 151.80, 145.52, 134.11, 133.56, 133.19, |
| | 131.79, 131.47, 130.96, 129.50, 128.25, 127.04, 124.93, 120.60, 116.53, |
| | 111.09, 101.26, 57.33, 44.22, 43.78, 41.42, 39.69, 37.61(2C), 30.62(2C), |
| | 25.66(2C) |
| E2 | ¹H NMR (300 MHz, Chloroform-d) δ 11.88 (s, 1H), 8.40 (d, J = 7.6 Hz, 1H), |
| | 8.29 (d, J = 10.3 Hz, 1H), 7.91 (q, J = 3.1 Hz, 1H), 7.71 (td, J = 7.2, 5.9, 3.8 Hz, |
| | 3H), 7.43 (t, J = 9.7 Hz, 1H), 7.21 (d, J = 5.1 Hz, 1H), 6.97-6.82 (m, 1H), 6.60 |
| | (s, 1H), 6.22 (d, J = 4.7 Hz, 1H), 4.75 (p, J = 8.6 Hz, 1H), 4.49 (t, J = 8.0 Hz, |
| | 1H), 4.13-3.89 (m, 2H), 3.72 (d, J = 6.0 Hz, 2H), 3.65-3.49 (m, 2H), 3.12 (s, |
| | 6H), 2.41 (d, J = 10.7 Hz, 2H), 2.23 (q, J = 6.8 Hz, 2H), 2.12 (s, 1H), 1.99 (d, |
| | J = 5.4 Hz, 2H), 1.64 (s, 2H), 0.99 (ddd, J = 6.7, 4.5, 1.8 Hz, 6H). ¹³C NMR (75 |
| | MHz, Chloroform-d) δ 171.89, 164.09, 162.99, 160.53, 158.42, 157.86, |
| | 152.85, 150.97, 145.39, 134.14, 134.10, 133.36, 133.04, 131.93, 131.24, |
| | 130.57, 129.46, 128.34, 127.11, 125.05, 121.45, 110.44, 101.70, 59.95, |
| | 59.87, 57.29, 41.66, 30.71, 30.47, 25.21 (2C), 25.14 (2C), 19.42 (2C), 18.64 |
| | (2C) |
| E3 | ¹H NMR (300 MHz, Chloroform-d) δ 11.05 (s, 1H), 8.55 (s, 1H), 8.40 (d, |
| | J = 2.2 Hz, 1H), 8.38 (t, J = 2.1 Hz, 1H), 8.18 (s, 1H), 7.98 (dd, J = 7.3, 2.4 Hz, |
| | 1H), 7.71 (d, J = 2.2 Hz, 3H), 7.69 (t, J = 1.9 Hz, 2H), 7.61 (dd, J = 10.2, 8.2 Hz, |
| | 1H), 7.29 (s, 1H), 7.12-7.06 (m, 1H), 7.04 (d, J = 2.5 Hz, 2H), 6.98 (s, 1H), |
| | 6.94 (d, J = 2.8 Hz, 1H), 6.25 (s, 1H), 4.88 (d, J = 7.3 Hz, 1H), 4.70 (t, J = 8.6 |
| | Hz, 1H), 4.16 (d, J = 5.1 Hz, 2H), 3.43 (dd, J = 14.2, 5.2 Hz, 2H), 3.31 (d, J = 5.2 |
| | Hz, 2H), 3.21 (dd, J = 14.3, 8.6 Hz, 2H), 3.12 (s, 7H), 2.34 (s, 2H), 1.95 (s, |
| | 2H), 1.79 (s, 2H), 1.60 (d, J = 6.8 Hz, 2H). ¹³C NMR (126 MHz, Chloroform- |
| | d) δ 171.60, 163.94, 162.94, 160.36, 159.87, 152.98, 146.80, 145.53, 136.19, |
| | 134.02, 133.57(2C), 133.32, 131.80, 131.45, 129.73, 129.48, 128.29, 127.33, |
| | 127.09, 124.91, 123.37, 122.10 (2C), 120.89, 119.59, 118.98, 111.33, 110.87, |
| | 110.70, 101.33, 57.42, 55.05, 41.42, 37.52, 30.58 (2C), 30.08, 29.70(2C), |
| | 28.71, 25.16 (2C) |
| E4 | ¹H NMR (300 MHz, Chloroform-d) δ 11.91 (s, 1H), 8.30 (dd, J = 7.2, 2.4 Hz, |
| | 1H), 7.94-7.74 (m, 2H), 7.62 (tdd, J = 8.3, 5.7, 2.7 Hz, 3H), 7.45 (ddd, J = 12.4, |
| | 7.5, 4.9 Hz, 1H), 7.11 (s, 5H), 6.79 (td, J = 10.8, 8.4 Hz, 1H), 6.13 (d, J = 12.3 |
| | Hz, 1H), 4.79 (q, J = 7.3 Hz, 1H), 4.65 (t, J = 8.5 Hz, 1H), 3.61-3.44 (m, 2H), |
| | 3.37 (d, J = 4.9 Hz, 2H), 3.14-3.07 (m, 2H), 3.05 (s, 6H), 2.29 (d, J = 16.6 Hz, |
| | 2H), 1.90 (s, 4H), 1.57 (s, 2H), 1.18 (s, 2H). ¹³C NMR (75 MHz, Chloroform- |
| | d) δ 170.46, 163.07, 161.82, 159.52, 157.26, 156.83 (d, J = 249 Hz), 151.71, |
| | 150.00, 144.27, 135.70, 135.65, 133.02, 132.30, 130.88, 130.18, 129.38, |
| | 128.39 (3C), 127.45 (2C), 127.26, 126.10, 125.79, 123.66, 119.84 (d, J = 12.1 |
| | Hz), 115.08, 109.15, 100.54, 56.25, 54.85, 40.61, 37.55 (2C), 36.05, 29.64 |
| | (2C), 24.15 (2C). |
| E5 | ¹H NMR (300 MHz, Chloroform-d) δ 11.05 (d, J = 15.7 Hz, 1H), 8.41 (d, |
| | J = 5.1 Hz, 2H), 7.96 (d, J = 7.2 Hz, 1H), 7.82-7.67 (m, 4H), 7.52 (dd, J = 11.4, |
| | 7.5 Hz, 1H), 6.96 (ddd, J = 11.7, 8.4, 3.9 Hz, 1H), 6.29 (d, J = 3.4 Hz, 1H), 6.09 |
| | (s, 1H), 4.86-4.69 (m, 2H), 4.15 (t, J = 9.1 Hz, 2H), 3.63 (s, 5H), 3.13 (s, 6H), |
| | 2.56 (q, J = 7.8 Hz, 2H), 2.40 (d, J = 10.4 Hz, 2H), 2.32-2.17 (m, 1H), 2.04 (s, |
| | 3H), 2.01 (s, 4H), 1.65 (s, 2H), 0.87 (d, J = 6.8 Hz, 2H). ¹³C NMR (75 MHz, |
| | Chloroform-d) δ 171.58, 164.10, 163.07, 161.13(d, J = 241.5 Hz), 160.66, |
| | 158.33, 152.90, 150.95, 145.47, 134.17, 133.48, 131.80, 131.36, 130.84, |
| | 129.47, 128.27, 127.09, 124.86, 121.01, 120.93 (d, J = 12.3 Hz), 116.31 (d, |
| | J = 22.1 Hz), 110.67, 101.48, 57.32, 53.22, 41.56, 37.33, 31.99, 30.70, 30.53, |
| | 30.15 (2C), 25.23 (2C), 25.19 (2C), 15.26. |
| E6 | ¹H NMR (300 MHz, DMSO-d₆) δ 12.61 (s, 1H), 8.53 (s, 1H), 8.25 (t, J = 8.1 |
| | Hz, 2H), 8.08 (s, 1H), 7.97 (d, J = 7.8 Hz, 1H), 7.86 (dt, J = 19.5, 7.2 Hz, 2H), |
| | 7.66 (d, J = 7.0 Hz, 1H), 7.48 (s, 1H), 7.28-7.17 (m, 1H), 6.90 (s, 1H), 6.46 (s, |
| | 1H), 4.68 (t, J = 8.5 Hz, 1H), 4.44 (t, J = 7.0 Hz, 1H), 4.34 (s, 2H), 3.03 (s, 6H), |
| | 2.51 (q, J = 1.9 Hz, 4H), 2.31 (s, 2H), 1.91 (s, 4H), 1.55 (s, 2H), 1.29 (d, J = 7.0 |
| | Hz, 3H). ¹³C NMR (75 MHz, DMSO-d₆) δ 172.48, 170.82, 163.65, 163.11, |
| | 159.88, 159.20, 157.13 (d, J = 246 Hz), 152.84, 152.50, 145.39, 134.87(d, |
| | J = 3.2 Hz), 133.99, 133.44 (d, J = 8.6 Hz), 132.01, 130.98 (d, J = 21.9 Hz), |
| | 129.53, 128.35, 126.53, 126.00, 123.36 (d, J = 13.9 Hz), 122.82, 116.84(d, |
| | J = 23.2 Hz), 110.55, 101.18, 60.22, 56.77, 49.38, 41.25, 40.79, 40.51, 40.24, |
| | 39.96, 39.82, 39.68, 39.53, 39.40, 39.12, 36.98, 30.55(2C), 25.14 (2C), |
| | 21.20, 19.03 (2C), 14.52. |
| E7 | ¹H NMR (300 MHz, DMSO-d₆) δ 12.59 (s, 1H), 9.89 (s, 1H), 8.73 (s, 1H), |
| | 8.64 (s, 1H), 8.33 (d, J = 5.1 Hz, 1H), 8.27-8.24 (m, 1H), 7.98-7.94 (m, 1H), |
| | 7.88 (td, J = 8.0, 7.6, 1.7 Hz, 1H), 7.82 (td, J = 7.4, 1.4 Hz, 1H), 7.66 (dd, J = 7.1, |
| | 2.3 Hz, 1H), 7.50 (ddd, J = 7.7, 4.8, 2.4 Hz, 1H), 7.23 (dd, J = 10.8, 8.4 Hz, |
| | 1H), 6.53 (s, 1H), 4.68 (q, J = 8.6 Hz, 1H), 4.34 (s, 2H), 3.03 (s, 6H), 2.32- |
| | 2.22 (m, 2H), 1.99 (s, 2H), 1.95-1.84 (m, 4H), 1.52 (s, 2H). ¹³C NMR (75 |
| | MHz, Chloroform-d) δ 174.66, 168.92, 163.99, 163.65, 160.77, 158.58, |
| | 158.05 (d, J = 236.25 Hz), 152.45, 151.33, 145.58, 134.09, 133.53, 132.65, |
| | 132.36, 131.75, 131.39, 129.45, 128.16, 126.96, 124.95, 120.59 (d, J = 11.8 |
| | Hz), 113.46 (d, J = 15.3 Hz), 100.65, 57.46, 42.38, 39.30 (2C), 37.55, 30.69 |
| | (2C), 25.02 (2C) |
| E8 | ¹H NMR (300 MHz, Chloroform-d) δ 11.30 (s, 1H), 9.47 (d, J = 24.4 Hz, 1H), |
| | 8.51 (d, J = 1.4 Hz, 1H), 8.50-8.44 (m, 1H), 8.33 (dt, J = 8.1, 2.4 Hz, 1H), 8.11 |
| | (t, J = 3.0 Hz, 1H), 7.61 (hept, J = 4.1, 3.6 Hz, 3H), 7.45 (s, 1H), 7.17 (d, J = 3.1 |
| | Hz, 1H), 6.78-6.71 (m, 1H), 6.27 (d, J = 2.8 Hz, 1H), 4.82 (d, J = 7.4 Hz, 1H), |
| | 4.69 (t, J = 8.6 Hz, 1H), 4.07 (d, J = 8.6 Hz, 1H), 3.81 (s, 2H), 3.04 (d, J = 10.4 |
| | Hz, 6H), 1.89-1.74 (m, 4H), 1.52-1.34 (m, 2H), 1.18 (s, 2H), 1.03 (d, J = 6.8 |
| | Hz, 6H). ¹³C NMR (75 MHz, Chloroform-d) δ 171.85, 164.07 (2C), 163.06, |
| | 160.51, 158.41, 152.85, 150.90, 145.37, 134.10, 133.35, 131.92, 131.23, |
| | 129.44, 128.33, 127.10 (2C), 124.80, 121.22, 121.02, 110.39, 101.58, 101.52, |
| | 59.95, 58.05, 41.69 (2C), 30.84, 30.47, 25.22 (2C), 19.42 (2C), 18.65 (2C). |
| E9 | ¹H NMR (300 MHz, DMSO-d₆) δ 12.58 (s, 1H), 10.81 (s, 1H), 10.23 (s, 1H), |
| | 8.80 (s, 1H), 8.66 (s, 1H), 8.26 (d, J = 7.2 Hz, 1H), 8.15 (dd, J = 7.6, 5.0 Hz, |
| | 1H), 7.91 (t, J = 8.7 Hz, 2H), 7.85-7.78 (m, 1H), 7.72 (d, J = 7.8 Hz, 1H), 7.47 |
| | (d, J = 6.0 Hz, 2H), 7.32 (d, J = 8.0 Hz, 1H), 7.26 (s, 1H), 7.20-7.11 (m, 1H), |
| | 7.03 (t, J = 7.8 Hz, 1H), 6.92 (t, J = 7.5 Hz, 1H), 6.54 (s, 1H), 4.93-4.81 (m, |
| | 1H), 4.74-4.59 (m, 1H), 4.29 (s, 2H), 3.41 (dd, J = 14.8, 3.6 Hz, 1H), 3.14 (dd, |
| | J = 14.8, 9.8 Hz, 1H), 3.02 (s, 6H), 2.27 (s, 2H), 1.75 (d, J = 27.3 Hz, 4H), 1.33 |
| | (s, 2H). ¹³C NMR (75 MHz, Chloroform-d) δ 176.05, 168.59, 164.93, 164.24, |
| | 157.38, 156.46, 149.58, 141.21, 139.07, 138.12, 136.47, 136.01, 134.27, |
| | 133.23, 132.53, 131.32, 129.91, 128.76, 126.02, 123.41, 117.33, 116.30, |
| | 114.48, 105.30, 62.18, 58.02, 42.36, 35.76 (2C), 35.13 (2C), 33.94, 29.37 |
| | (2C) |
| E10 | ¹H NMR (300 MHz, Chloroform-d) δ 10.62 (s, 1H), 8.67 (s, 1H), 8.57 (s, |
| | 1H), 8.43 (dd, J = 7.1, 2.2 Hz, 1H), 7.99 (d, J = 7.3 Hz, 1H), 7.78-7.66 (m, 4H), |
| | 7.36-7.30 (m, 2H), 7.28 (d, J = 2.0 Hz, 2H), 7.23 (s, 2H), 7.20 (d, J = 4.0 Hz, |
| | 1H), 7.00 (dd, J = 11.4, 8.4 Hz, 1H), 6.38 (s, 1H), 5.13-5.01 (m, 1H), 4.71 (q, |
| | J = 8.6 Hz, 1H), 4.25 (s, 2H), 3.26 (dd, J = 7.0, 3.5 Hz, 2H), 3.12 (s, 6H), 2.32 |
| | (s, 2H), 1.98-1.87 (m, 4H), 1.54 (s, 2H). ¹³C NMR (75 MHz, Chloroform-d) |
| | δ 170.19, 164.04, 163.22, 160.52, 158.49, 152.50, 151.46, 145.47, 136.34 |
| | (2C), 134.21, 133.55, 132.38, 131.80, 131.44, 129.47, 129.37 (2C), 128.61 |
| | (2C), 128.30, 127.08, 127.01, 124.97, 116.62, 116.28, 113.51, 100.83, 57.48, |
| | 53.79, 39.38, 38.27 (2C), 37.63, 30.67 (2C), 24.87 (2C) |
| E11 | ¹H NMR (300 MHz, Chloroform-d) δ 11.66 (s, 1H), 9.58 (s, 1H), 8.59 (s, |
| | 1H), 8.41 (dt, J = 6.3, 1.6 Hz, 1H), 8.01 (dd, J = 7.2, 2.4 Hz, 1H), 7.72-7.68 (m, |
| | 3H), 7.64-7.53 (m, 2H), 7.35-7.29 (m, 1H), 6.96 (dd, J = 11.5, 8.5 Hz, 1H), |
| | 6.36 (s, 1H), 5.13 (d, J = 7.3 Hz, 1H), 4.75 (t, J = 8.6 Hz, 1H), 4.22 (s, 2H), 3.11 |
| | (s, 6H), 2.75-2.65 (m, 2H), 2.39-2.24 (m, 4H), 2.26-2.13 (m, 2H), 2.08 (s, |
| | 3H), 1.91 (s, 4H), 1.54 (s, 2H). ¹³C NMR (75 MHz, Chloroform-d) δ 174.66, |
| | 168.92, 163.99, 163.65, 160.77, 158.58, 158.05, 152.45, 151.33, 145.58, |
| | 134.09, 133.53, 132.65, 132.36, 131.39, 129.45, 128.16, 126.96, 124.95, |
| | 120.51, 113.56, 113.36, 100.65, 57.46, 42.38, 39.30, 37.55 (2C), 35.15, 30.69 |
| | (2C), 25.02 (2C). |
| E12 | ¹H NMR (300 MHz, DMSO-d₆) δ 12.61 (s, 1H), 10.01 (s, 1H), 8.74 (s, 1H), |
| | 8.64 (s, 1H), 8.26 (dd, J = 7.5, 1.6 Hz, 1H), 8.21 (t, J = 6.4 Hz, 1H), 7.99-7.94 |
| | (m, 1H), 7.86 (dtd, J = 18.3, 7.2, 1.5 Hz, 3H), 7.65 (dd, J = 7.1, 2.4 Hz, 1H), |
| | 7.51 (td, J = 5.2, 2.5 Hz, 1H), 7.23 (dd, J = 10.8, 8.5 Hz, 1H), 6.54 (s, 1H), 4.65 |
| | (dt, J = 15.7, 7.7 Hz, 2H), 4.34 (s, 2H), 3.04 (d, J = 5.8 Hz, 6H), 2.24 (d, J = 10.0 |
| | Hz, 2H), 1.78 (d, J = 8.2 Hz, 4H), 1.42 (d, J = 7.0 Hz, 5H). ¹³C NMR (75 MHz, |
| | Chloroform-d) δ 171.84, 164.06, 162.97 (d, J = 3.2 Hz), 161.40, 160.70, |
| | 158.70, 157.93(d, J = 246.75 Hz), 152.55, 151.47, 145.54, 134.22, 133.56, |
| | 132.29, 131.81, 131.43, 129.48, 128.26, 127.04, 124.97, 116.64, 116.47 (d, |
| | J = 25.6 Hz), 113.40, 100.79, 57.50, 48.17, 37.62 (2C), 35.16, 30.55, 25.09 |
| | (2C), 24.75 (2C), 18.63. |

### Example 11

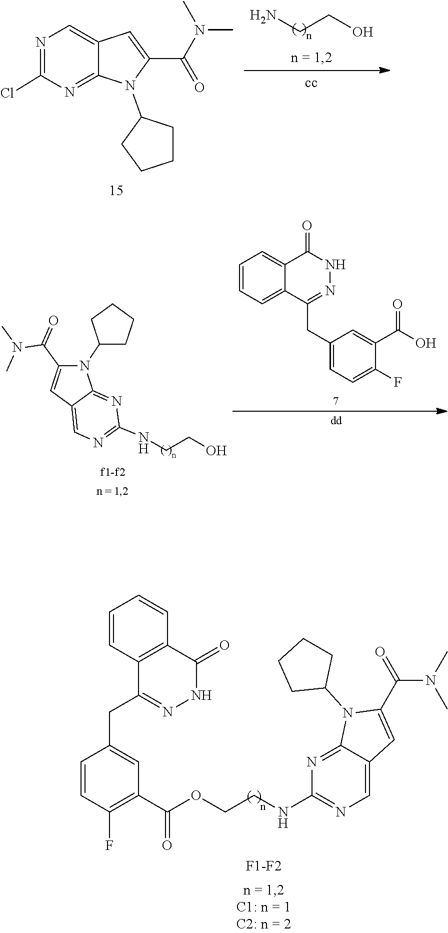

Synthesis of intermediates f1-f2: Compound 15 (1 mmol) and ethanolamine or propanolamine (1 mmol) were dissolved in 50 mL of n-butanol and a catalytic amount of concentrated hydrochloric acid was added. The reaction mixture was reacted at 110° C. overnight. The reaction was monitored by TLC and confirmed to be complete. After completion of the reaction, the reaction mixture was poured into a saturated sodium bicarbonate solution and extracted with dichloromethane. An organic phase was collected, dried over anhydrous Na₂SO₄, and concentrated under reduced pressure. A crude product was purified by column chromatography to yield the intermediates f1-f2.

Synthesis of target compounds F1-F2: Compounds f1-f2 (0.5 mmol) and intermediate 13 (0.5 mmol) were dissolved in 50 mL of DCM, and EDCI (1 mmol) and DMAP (0.05 mmol) were added. The reaction mixture was reacted at room temperature overnight. The reaction was monitored by TLC and confirmed to be complete. After completion of the reaction, the reaction mixture was poured into a saturated ammonium chloride solution and extracted with dichloromethane. An organic phase was collected, dried over anhydrous Na₂SO₄, and concentrated under reduced pressure. A crude product was purified by column chromatography to yield the target compounds F1-F2.

Table 6 shows spectroscopic data of compounds F1-F2.

TABLE 6

| Serial | |
|---|---|
| No. | Spectroscopic data |
| F1 | ¹H NMR (300 MHz, Chloroform-d) δ 11.34 (s, 1H), 8.61 (s, 1H), 8.46 (d, |
| | J = 5.9 Hz, 1H), 8.00 (d, J = 6.4 Hz, 1H), 7.75 (d, J = 7.1 Hz, 3H), 7.42 (s, 1H), |
| | 7.04 (t, 1H), 6.37 (s, 1H), 4.93-4.69 (m, 1H), 4.70-4.47 (m, 2H), 4.27 (s, 1H), |
| | 3.87 (d, J = 10.4 Hz, 2H), 3.15 (s, 6H), 2.46 (s, 2H), 2.03 (s, 4H), 1.67 (s, 2H), |
| | 1.14 (t, J = 5.5 Hz, 1H). ¹³C NMR (75 MHz, Chloroform-d) δ 164.30, 164.16, |
| | 162.66(d, J = 261 Hz), 160.66, 158.41, 152.89, 151.99, 145.39, 134.61, |
| | 133.54, 133.36 (d, J = 3.8 Hz), 132.30, 131.46, 130.88, 129.51, 128.37, |
| | 127.13, 124.84, 118.64 (d, J = 9.9 Hz), 117.29 (d, J = 23.0 Hz), 111.27, 101.25, |
| | 63.97, 57.35, 45.96, 40.70 (2C), 37.39, 30.64 (2C), 25.29 (2C). |
| F2 | ¹H NMR (300 MHz, Chloroform-d) δ 10.92 (s, 1H), 8.53 (s, 1H), 8.48-8.43 |
| | (m, 1H), 7.92 (dd, J = 6.8, 2.5 Hz, 1H), 7.75 (dq, J = 6.0, 4.9, 4.1 Hz, 3H), 7.41 |
| | (ddd, J = 7.1, 4.4, 2.5 Hz, 1H), 7.06 (dd, J = 10.4, 8.5 Hz, 1H), 6.35 (s, 1H), |
| | 4.75 (t, J = 8.6 Hz, 1H), 4.47 (t, J = 6.1 Hz, 2H), 4.30 (s, 2H), 3.63 (q, J = 6.4 |
| | Hz, 2H), 3.14 (s, 6H), 2.45 (q, J = 8.5, 7.9 Hz, 2H), 2.13 (p, J = 6.4 Hz, 2H), |
| | 2.01 (d, J = 12.2 Hz, 4H), 1.61 (d, J = 5.8 Hz, 2H). ¹³C NMR (75 MHz, |
| | Chloroform-d) δ 164.37, 162.60(d, J = 135 Hz), 160.48, 158.72, 153.06, |
| | 151.79, 145.44, 134.44 (d, J = 8.9 Hz), 133.58, 133.42, 132.15, 131.52, |
| | 130.68, 129.54, 128.41, 127.18, 124.92, 118.87 (d, J = 10.5 Hz), 117.37 (d, |
| | J = 22.9 Hz), 111.01, 101.25, 77.25, 63.59, 57.35, 38.87 (2C), 37.61, 30.59 |
| | (2C), 28.49, 25.22 (2C) 37.61, 30.59 (2C), 28.49, 25.22 (2C) |

The effectiveness of the present application was verified through the following tests:
(1) IC₅₀ values of the dual-target inhibitors (Examples 1-11) against CDK4 were determined. Results are shown in Table 7 (* indicates IC₅₀>500 nM, ** indicates 100 nM<IC₅₀<500 nM, *** indicates 10 nM<IC₅₀<100 nM, and **** indicates IC₅₀<10 nM).
(2) The IC₅₀ values of the dual-target inhibitors (Examples 1-11) against PARP1 were determined. Results are shown in Table 7 (#indicates IC₅₀>500 nM, ##indicates 100 nM<IC₅₀<500 nM, ###indicates 10 nM<IC₅₀<100 nM, and ####indicates IC₅₀<10 nM).

TABLE 7
IC₅₀ values of dual-target inhibitors for CDK4
and PARP1 in the compounds of the examples.

IC₅₀

| Compound | CDK4 | PARP1 |
|---|---|---|
| A1 | **** | ## |
| A2 | **** | # |
| A3 | *** | ### |
| A4 | **** | ## |
| A5 | *** | # |
| B1 | **** | # |
| C1 | *** | ## |
| C2 | *** | # |
| C3 | *** | # |
| C4 | **** | ### |
| C5 | **** | #### |
| C6 | **** | ### |
| C7 | **** | ### |
| C8 | *** | ### |
| C9 | **** | ### |
| D1 | * | ## |
| D2 | * | ### |
| D3 | ** | #### |
| D4 | * | #### |
| D5 | * | #### |
| D6 | * | #### |
| D7 | * | ### |
| D8 | * | # |
| D9 | * | #### |
| D10 | * | ### |
| D11 | *** | #### |
| D12 | **** | #### |
| D13 | *** | ### |
| D14 | **** | ### |
| E1 | ** | #### |
| E2 | ** | ### |
| E3 | * | ### |
| E4 | * | ### |
| E5 | ** | ### |
| E6 | * | ## |
| E7 | * | #### |
| E8 | * | ### |
| E9 | * | ### |
| E10 | * | ## |
| E11 | * | ## |
| E12 | * | ### |
| F1 | * | ### |
| F2 | * | ### |

The molecular-level data on inhibitory activity of dual-target inhibitor compounds against both CDK4 and PARP1 provided in the examples of the present application demonstrate that compounds C4-C9 and D11-D14 exhibit strong inhibitory activity against both CDK4 and PARP1.

In conclusion, the present application provides the CDK4/PARP1 dual-target inhibitor. The CDK4/PARP1 dual-target inhibitor described in the present application can effectively overcome the shortcomings of poor efficacy of single CDK4 inhibitors and weak activity of single PARP1 inhibitors, exhibits strong inhibitory effects on both CDK4 and PARP1, and has promising application prospects.

The above embodiments are provided merely for illustrating the technical solutions of the present application and are not intended to limit the scope of protection of the present application. It is apparent that the described embodiments are not all embodiments but only part of embodiments of the present application. All other embodiments obtained by those of ordinary skill in the art on the basis of the embodiments without creative work fall within the scope of protection of the present application. While the present application has been described in detail with reference to the aforementioned embodiments, it should be understood by those skilled in the art that without departing from the inventive concept and without creative effort, various modifications may be made by combining, adding, deleting, or otherwise adjusting the features of the embodiments herein, depending on specific circumstances. All such derived technical solutions, which differ in form but remain consistent in essence with the core principles of the present application, shall fall within the scope of protection of the present application.

What is claimed is:

1. A CDK4/PARP1 dual-target inhibitor, wherein the CDK4/PARP1 dual-target inhibitor is a compound having a general structural formula as shown in formula 1, or a pharmaceutically acceptable salt, solvate, hydrate or prodrug thereof, 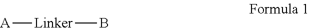
wherein A of the compound is selected from: 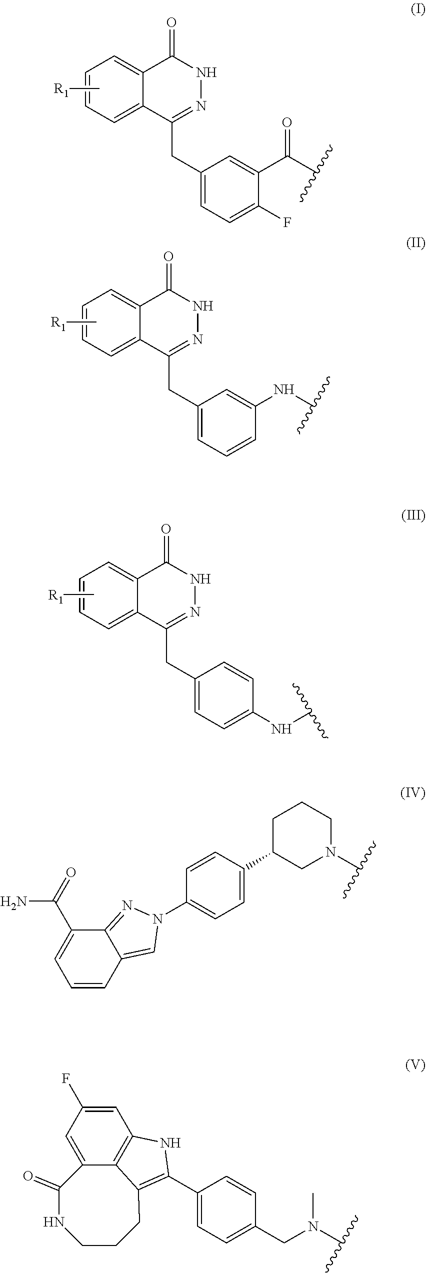
wherein R₁ is selected from H, 1-F, 2-F, 3-F, 6-F, 1-Cl, 2-Cl, 3-Cl, 6-Cl, 1-Br, 2-Br, 3-Br, 6-Br, 1-I, 2-I, 3-I, 6-I, 1-I, 2-I, 3-I, 6-I, 1-NO₂, 2-NO₂, 3-NO₂, 6-NO₂, 1-CH₃, 2-CH₃, 3-CH₃, 6-CH₃, 1-OCH₃, 2-OCH₃, 3-OCH₃, and 6-OCH₃; and
when A is selected from (I), Linker is selected from the following groups: 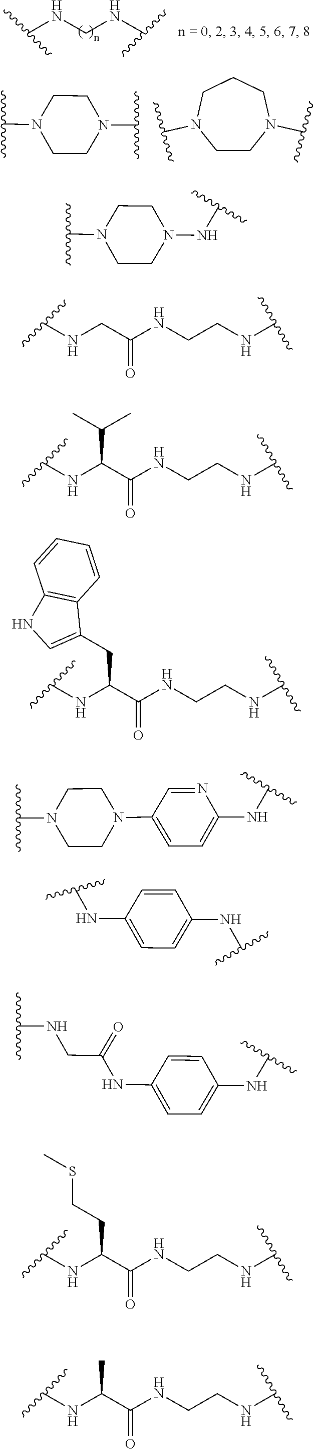 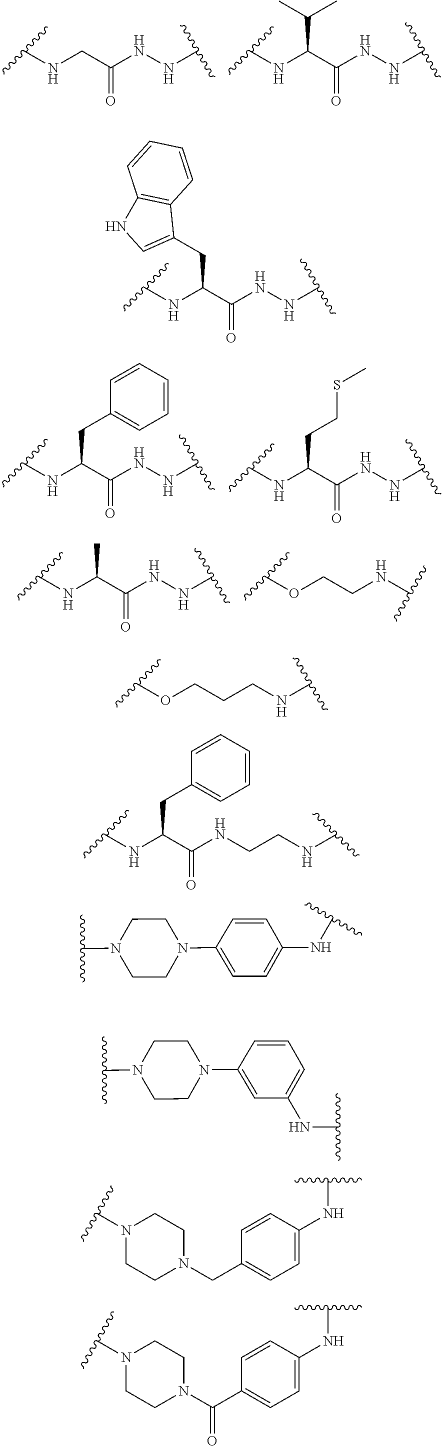 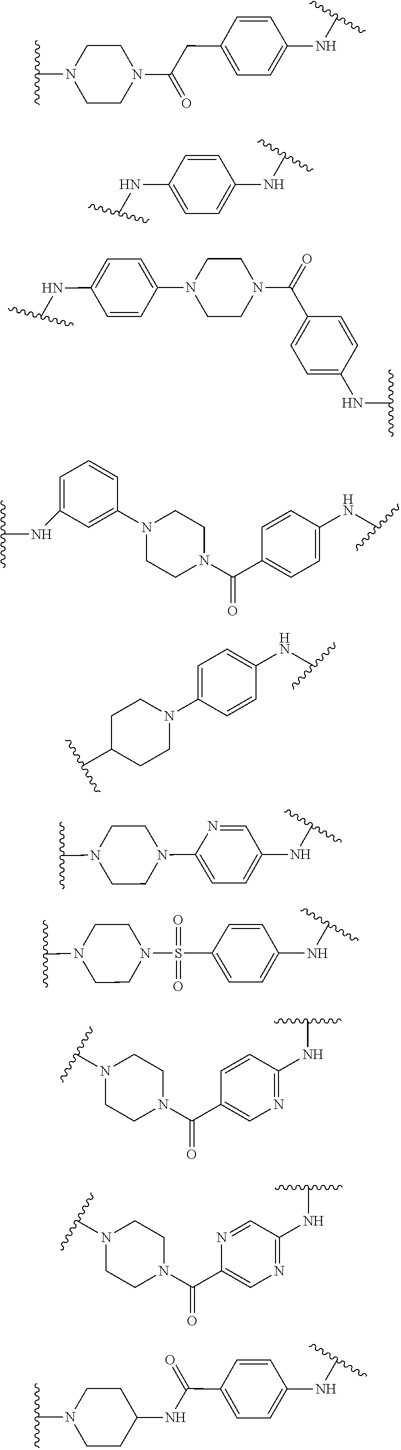 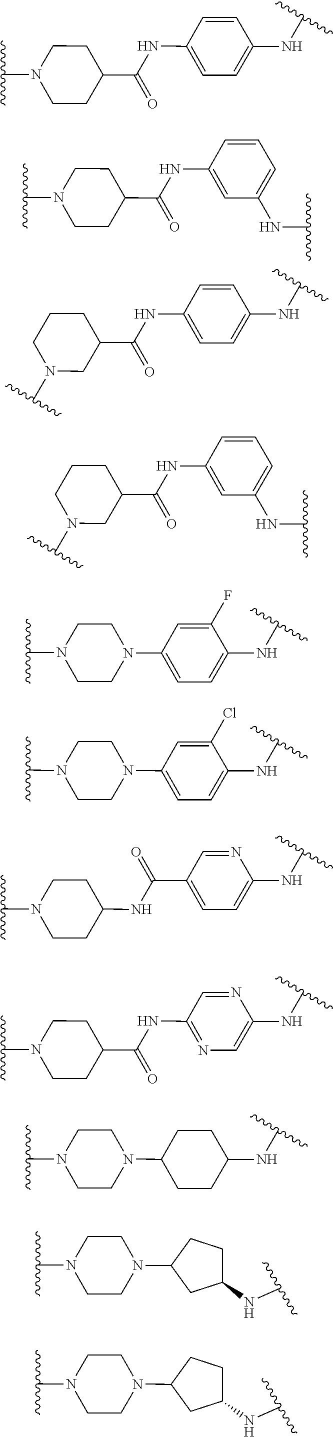 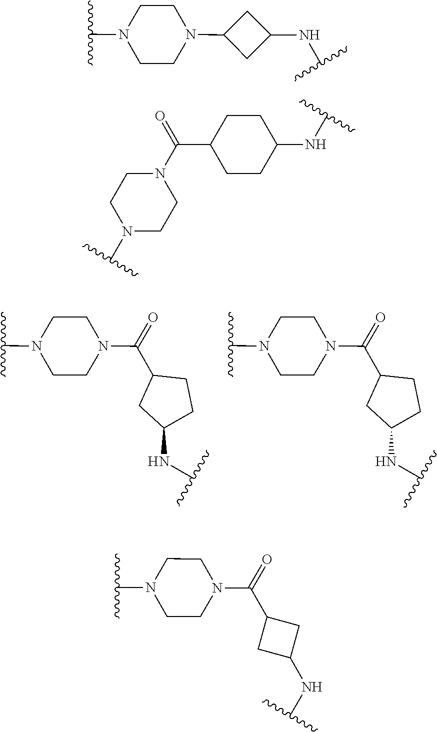 [SMILES: *NC1CC(N2CCN(*)CC2)C1 ; *NC1CCC(C(=O)N2CCN(*)CC2)CC1 ; *N[C@@H]1CCC(C(=O)N2CCN(*)CC2)C1 ; *N[C@H]1CCC(C(=O)N2CCN(*)CC2)C1 ; *NC1CC(C(=O)N2CCN(*)CC2)C1]
when A is selected from (II), (III), (IV), and (V), Linker is selected from the following groups: 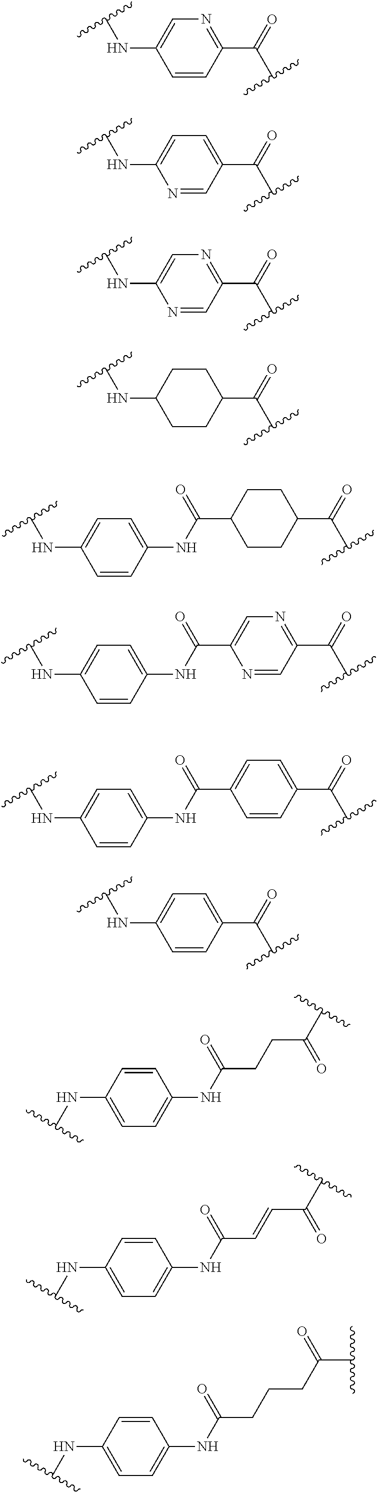
wherein B of the compound is selected from the following groups:

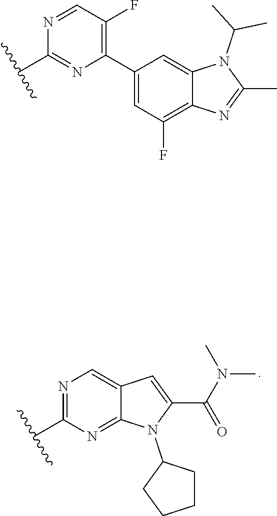 [SMILES: *c1ncc(F)c(-c2cc(F)c3nc(C)n(C(C)C)c3c2)n1 ; *c1ncc2cc(C(=O)N(C)C)n(C3CCCC3)c2n1]

2. The CDK4/PARP1 dual-target inhibitor according to claim 1, wherein the inhibitor is an enantiomer of formula 1, a mixture of enantiomers, a mixture of two or more diastereomers, a tautomer, a mixture of two or more tautomers, or an isotopically labeled variant of formula 1.

3. The CDK4/PARP1 dual-target inhibitor according to claim 1, wherein the inhibitor is selected from the following structures:

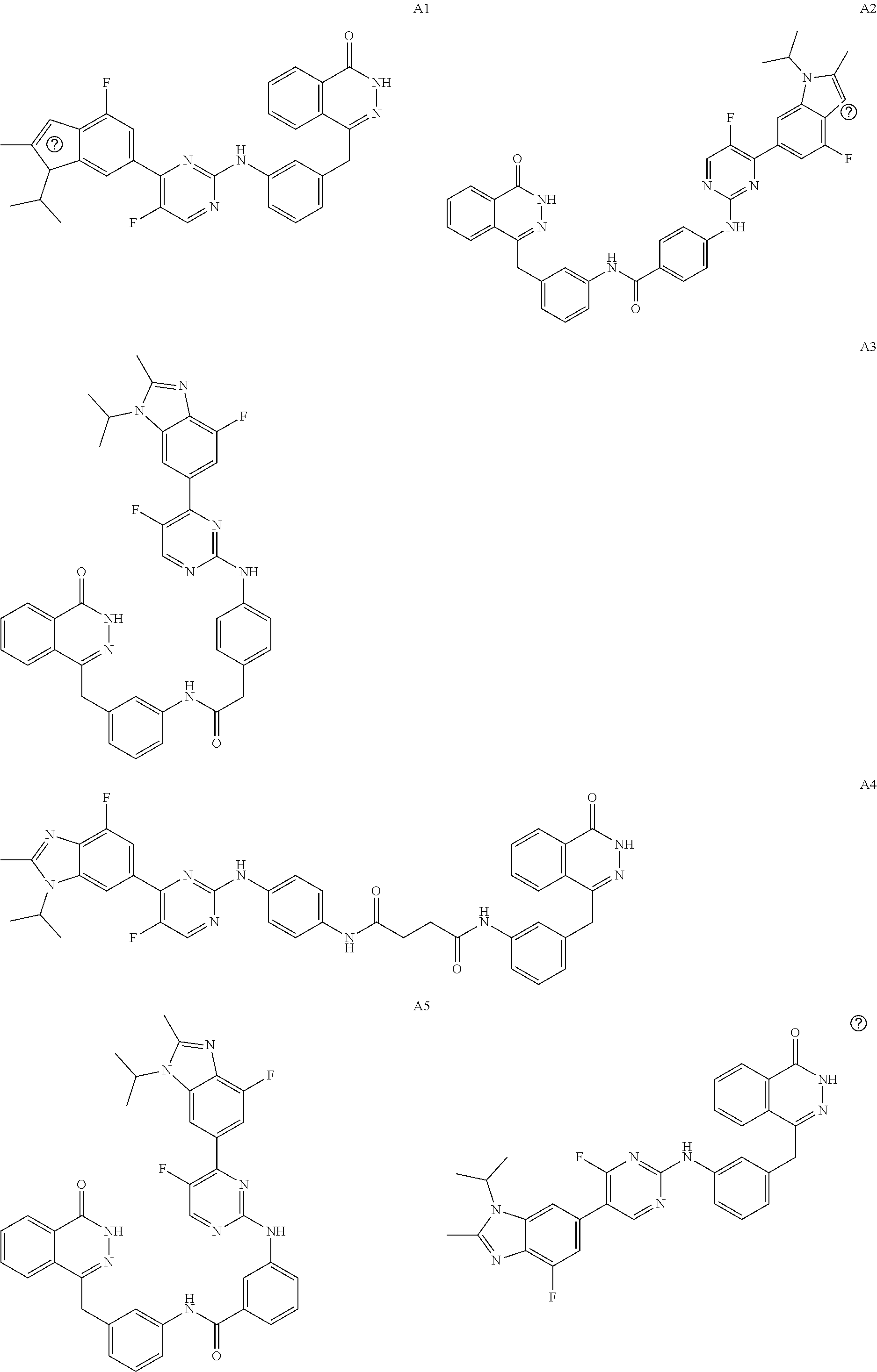

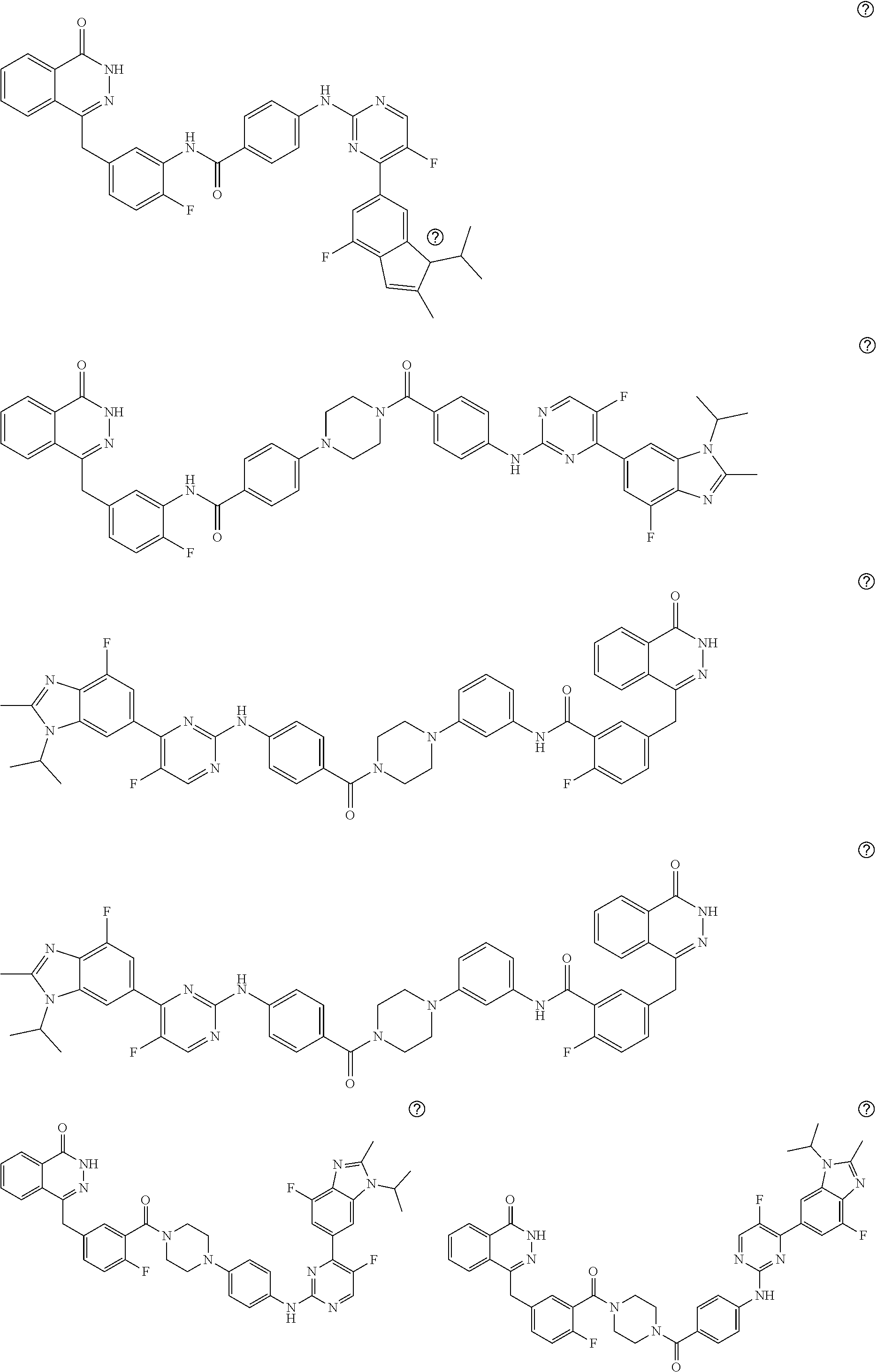

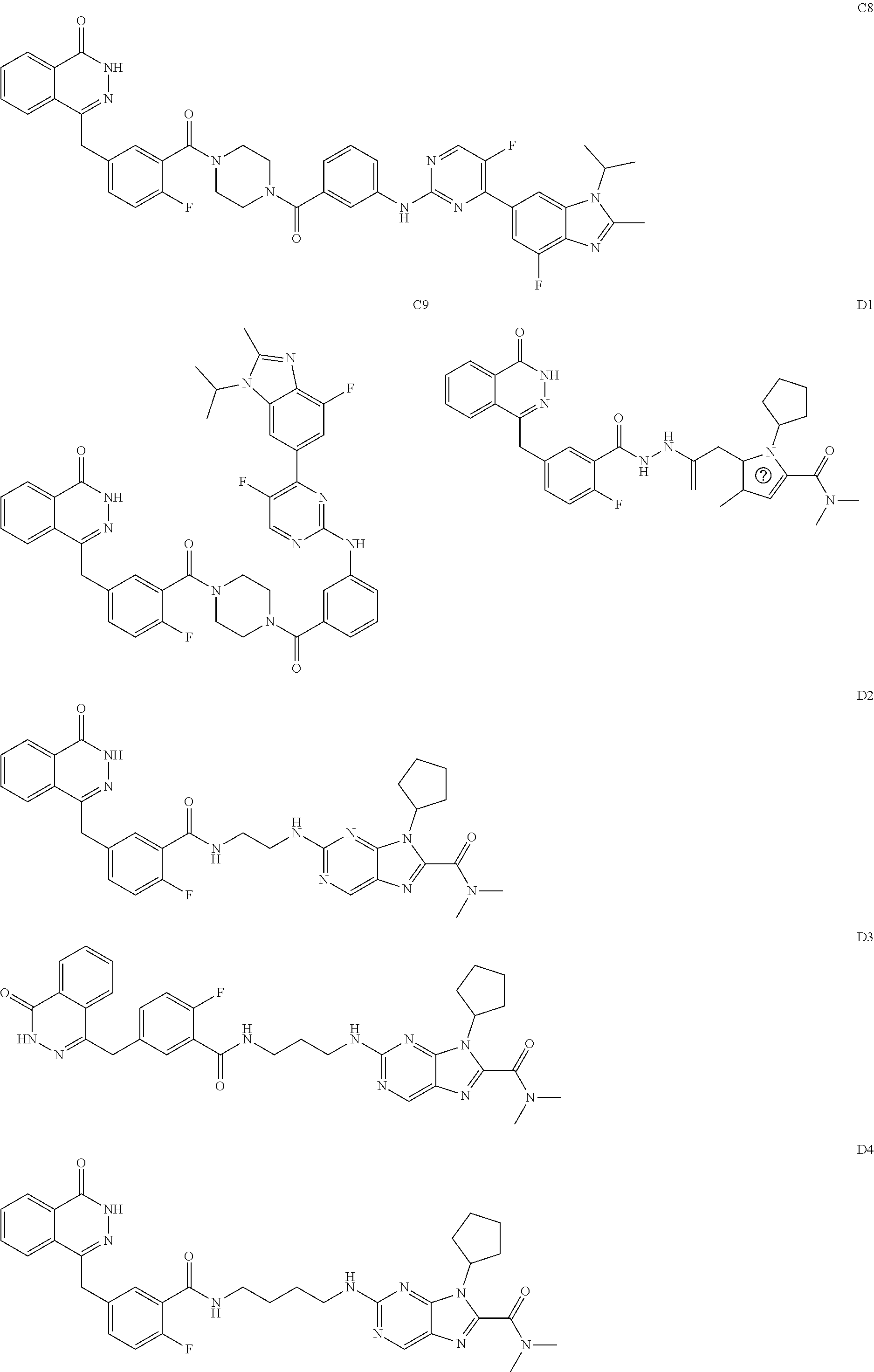

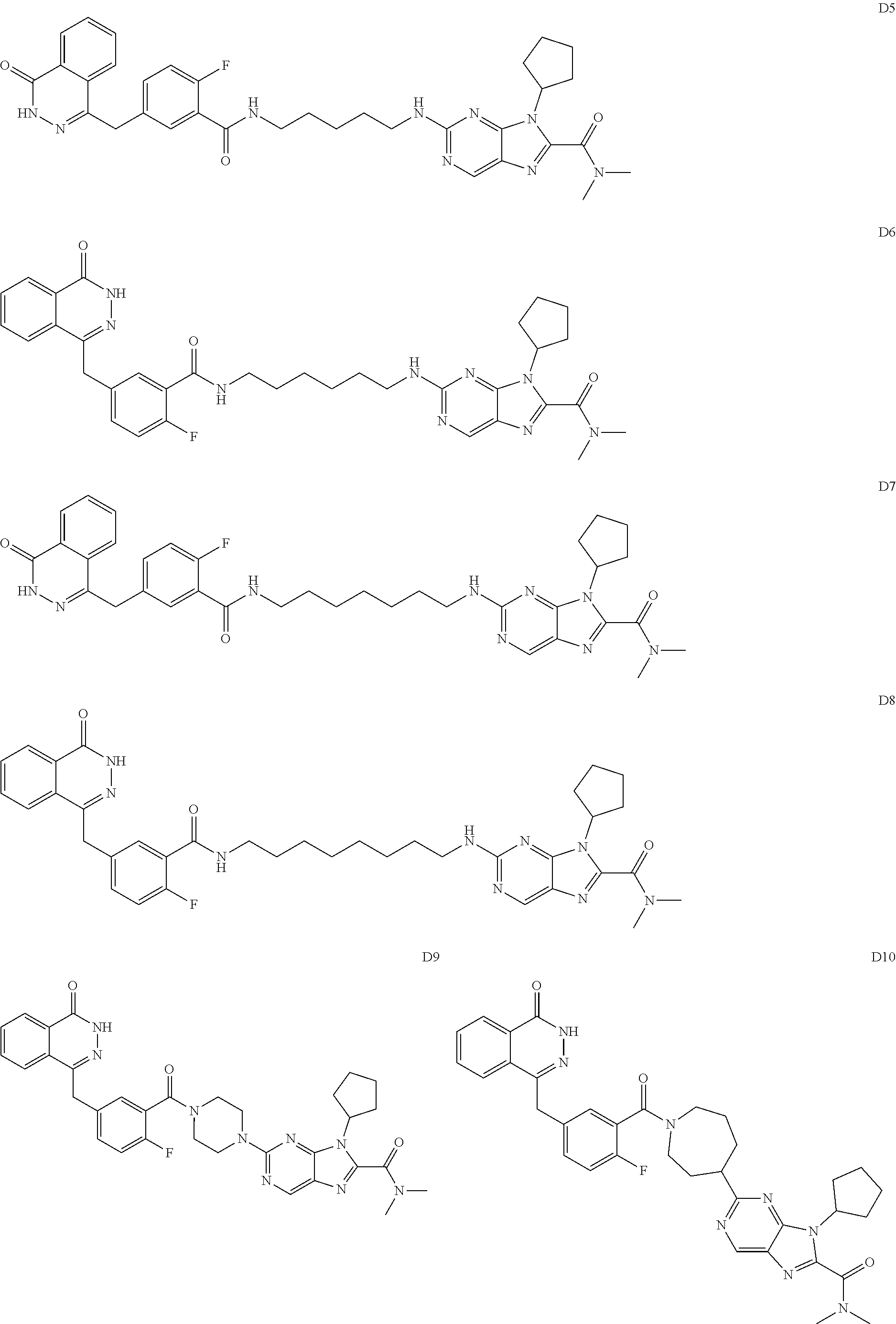 [SMILES: CN(C)C(=O)c1nc2cnc(NCCCCCNC(=O)c3cc(Cc4n[nH]c(=O)c5ccccc45)ccc3F)nc2n1C1CCCC1 ; CN(C)C(=O)c1nc2cnc(NCCCCCCNC(=O)c3cc(Cc4n[nH]c(=O)c5ccccc45)ccc3F)nc2n1C1CCCC1 ; CN(C)C(=O)c1nc2cnc(NCCCCCCCNC(=O)c3cc(Cc4n[nH]c(=O)c5ccccc45)ccc3F)nc2n1C1CCCC1 ; CN(C)C(=O)c1nc2cnc(NCCCCCCCCNC(=O)c3cc(Cc4n[nH]c(=O)c5ccccc45)ccc3F)nc2n1C1CCCC1 ; CN(C)C(=O)c1nc2cnc(N3CCN(C(=O)c4cc(Cc5n[nH]c(=O)c6ccccc56)ccc4F)CC3)nc2n1C1CCCC1 ; CN(C)C(=O)c1nc2cnc(C3CCCN(C(=O)c4cc(Cc5n[nH]c(=O)c6ccccc56)ccc4F)CC3)nc2n1C1CCCC1]

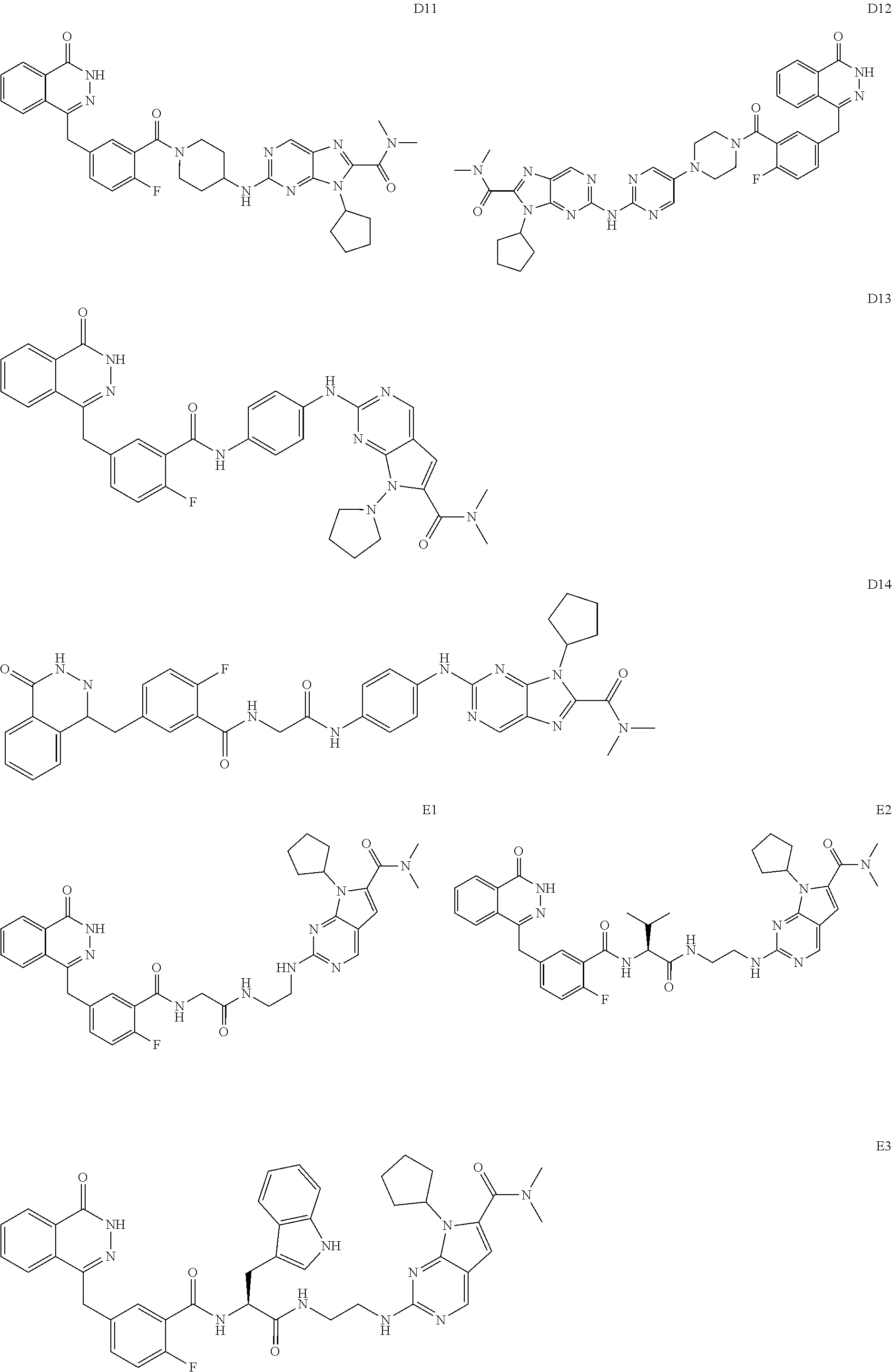

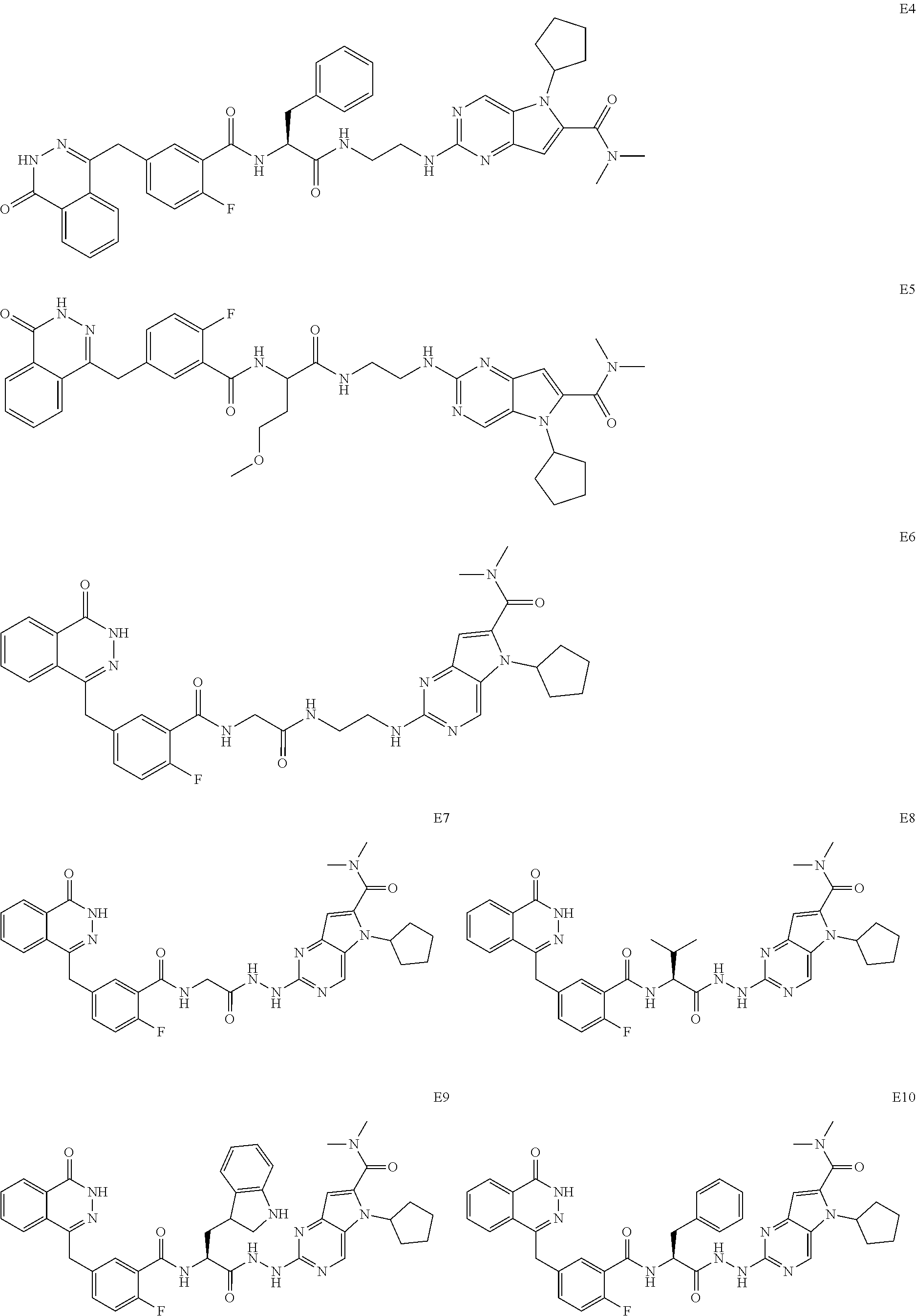

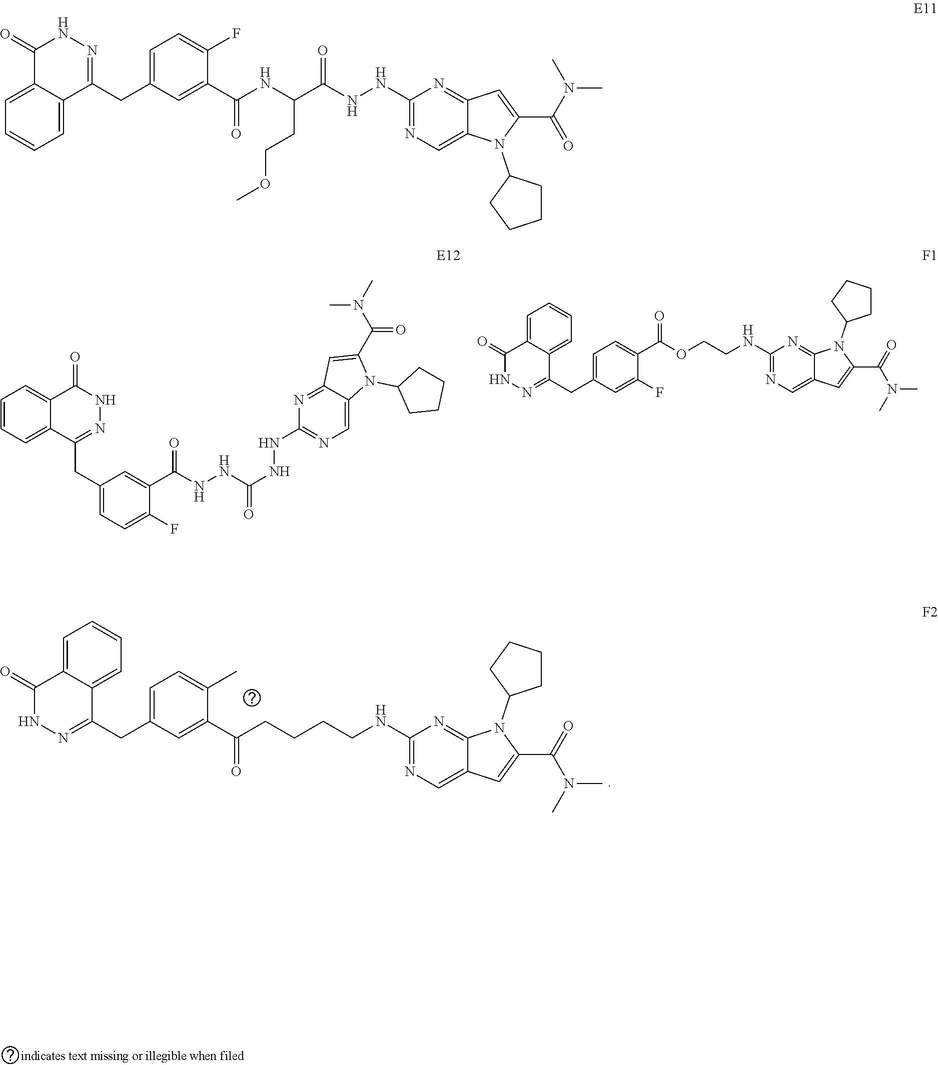 [SMILES: COCCC(NC(=O)c1cc(Cc2n[nH]c(=O)c3ccccc23)ccc1F)C(=O)NNc1ncc2c(cc(C(=O)N(C)C)n2C2CCCC2)n1 ; CN(C)C(=O)c1cc2nc(NNC(=O)NNC(=O)c3cc(Cc4n[nH]c(=O)c5ccccc45)ccc3F)ncc2n1C1CCCC1 ; CN(C)C(=O)c1cc2cnc(NCCOC(=O)c3ccc(Cc4n[nH]c(=O)c5ccccc45)cc3F)nc2n1C1CCCC1 ; Cc1ccc(Cc2n[nH]c(=O)c3ccccc23)cc1C(=O)CCCCNc1ncc2cc(C(=O)N(C)C)n(C3CCCC3)c2n1]

4. Use of the CDK4/PARP1 dual-target inhibitor according to claim 1 in preparation of a medicament for treating, ameliorating or preventing diseases mediated by poly(ADP-ribose) polymerase (PARP), cyclin-dependent kinase 4 (CDK4), and cyclin-dependent kinase 6 (CDK6).

5. A pharmaceutical composition, comprising one or more CDK4/PARP1 dual-target inhibitors wherein the CDK4/PARP1 dual-target inhibitor is a compound having a general structural formula as shown in formula 1, or a pharmaceutically acceptable salt, solvate, hydrate or prodrug thereof, 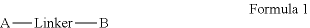
wherein A of the compound is selected from: 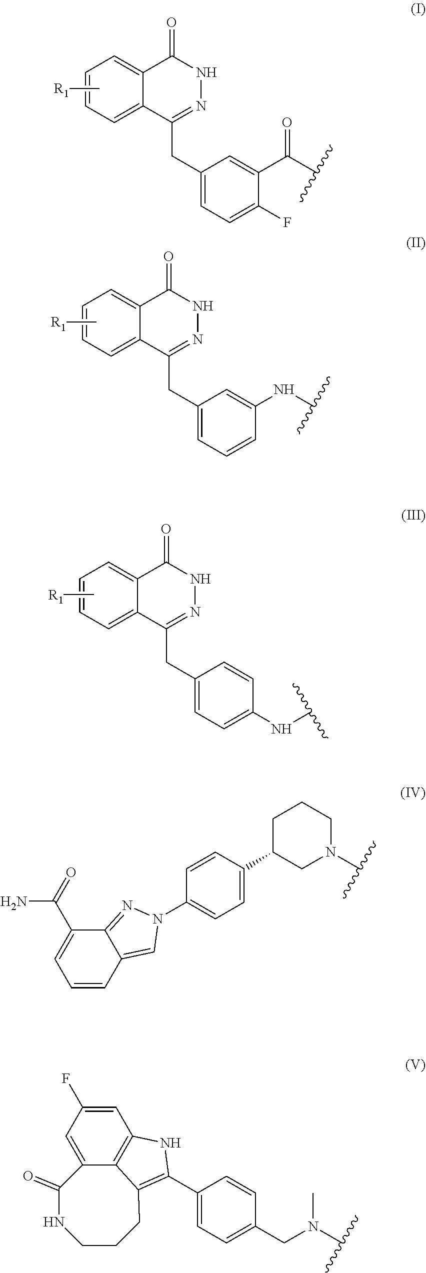
wherein R₁ is selected from H, 1-F, 2-F, 3-F, 6-F, 1-Cl, 2-Cl, 3-Cl, 6-Cl, 1-Br, 2-Br, 3-Br, 6-Br, 1-I, 2-I, 3-I, 6-I, 1-I, 2-I, 3-I, 6-I, 1-NO₂, 2-NO₂, 3-NO₂, 6-NO₂, 1-CH₃, 2-CH₃, 3-CH₃, 6-CH₃, 1-OCH₃, 2-OCH₃, 3-OCH₃, and 6-OCH₃; and
when A is selected from (I), Linker is selected from the following groups: 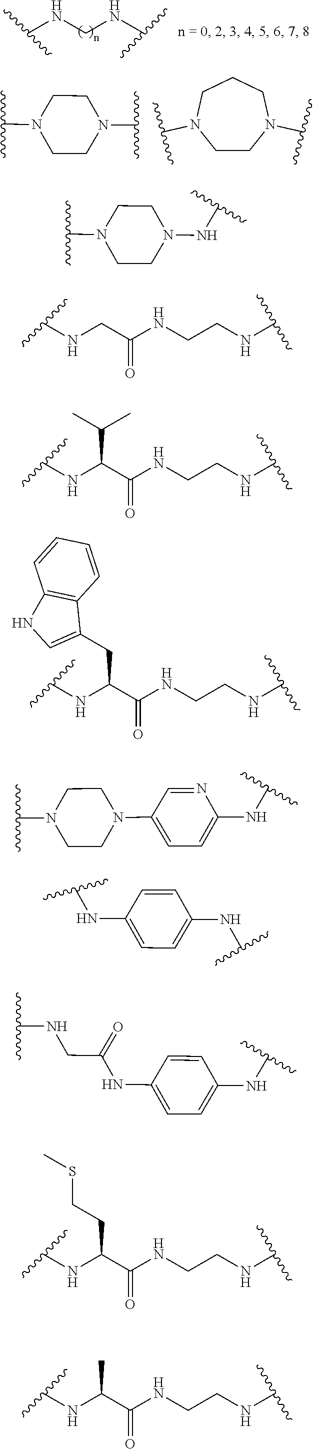 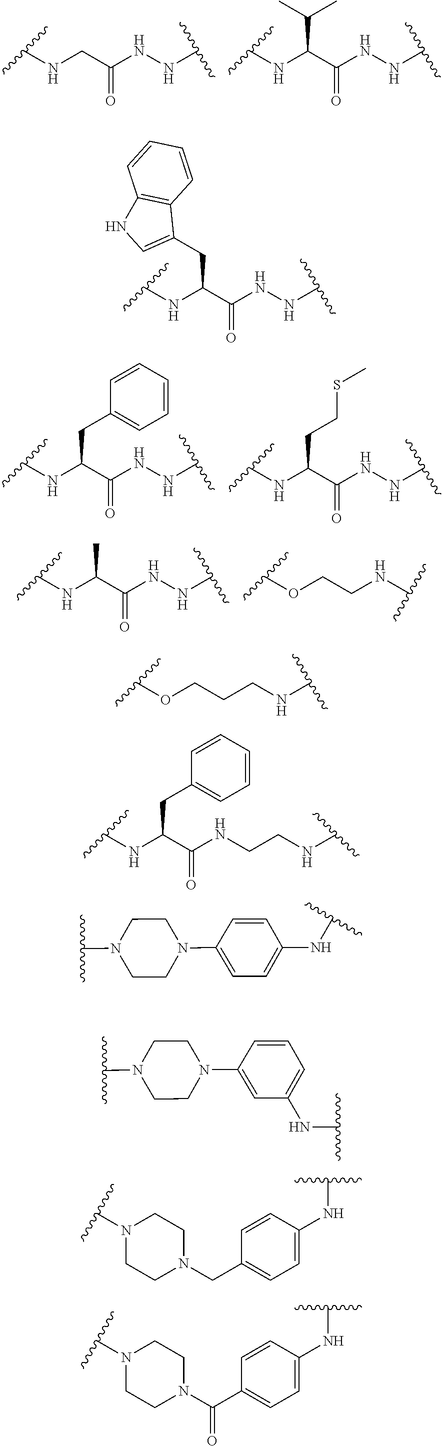 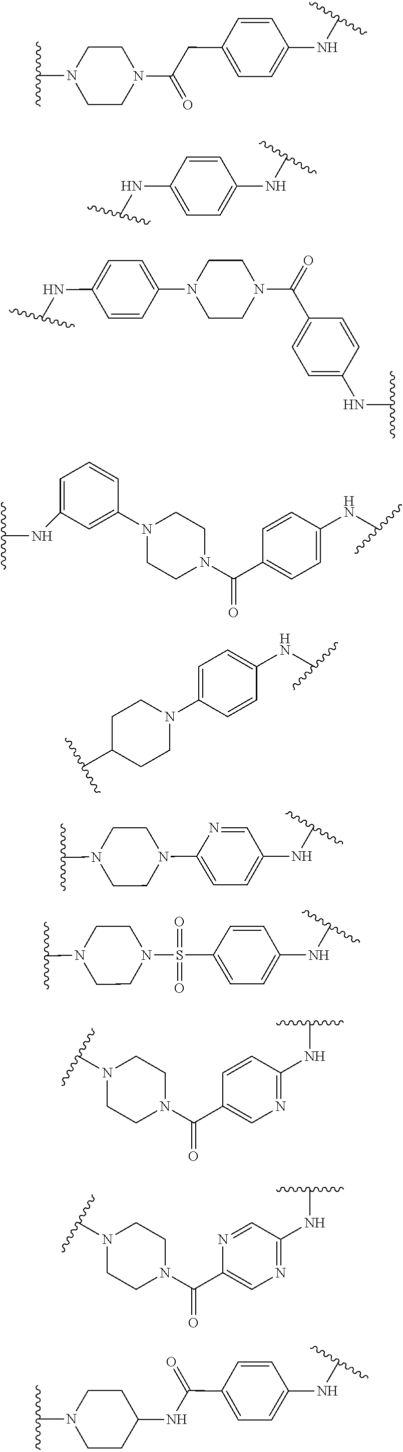 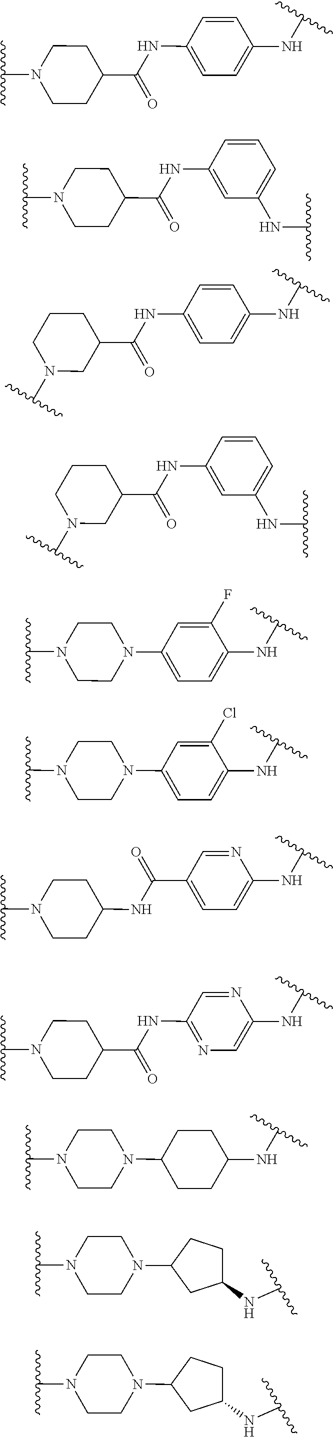 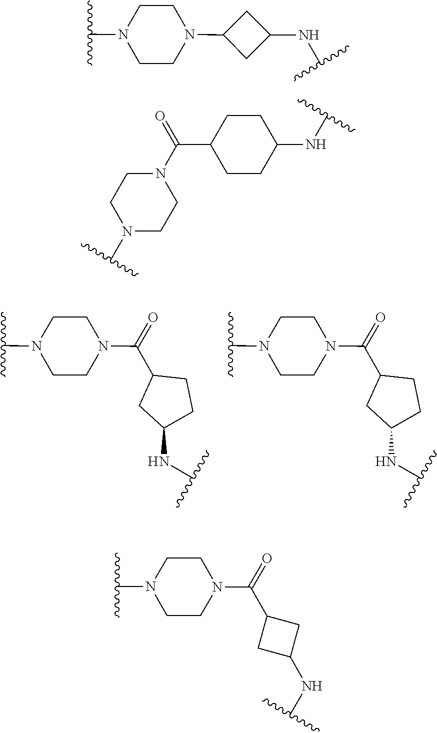 [SMILES: *NC1CC(N2CCN(*)CC2)C1 ; *NC1CCC(C(=O)N2CCN(*)CC2)CC1 ; *N[C@@H]1CCC(C(=O)N2CCN(*)CC2)C1 ; *N[C@H]1CCC(C(=O)N2CCN(*)CC2)C1 ; *NC1CC(C(=O)N2CCN(*)CC2)C1]
when A is selected from (II), (III), (IV), and (V), Linker is selected from the following groups: 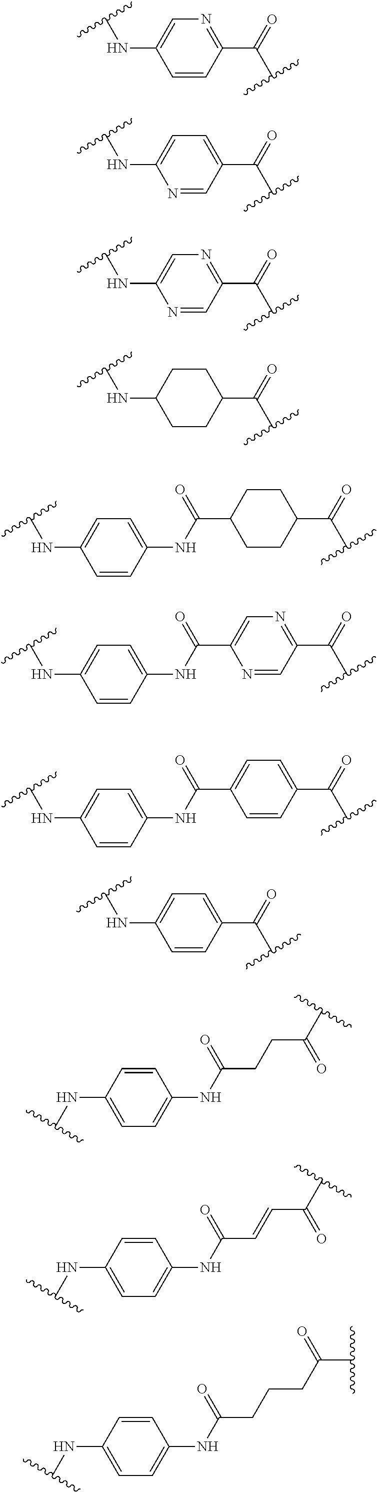
wherein B of the compound is selected from the following groups:

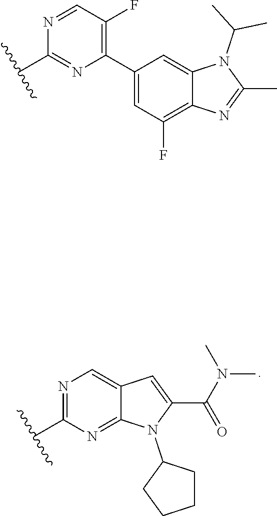 [SMILES: *c1ncc(F)c(-c2cc(F)c3nc(C)n(C(C)C)c3c2)n1 ; *c1ncc2cc(C(=O)N(C)C)n(C3CCCC3)c2n1]

6. A pharmaceutical composition, comprising one or more CDK4/PARP1 dual-target inhibitors wherein the CDK4/PARP1 dual-target inhibitor is a compound having a general structural formula as shown in formula 1, or a pharmaceutically acceptable salt, solvate, hydrate or prodrug thereof, 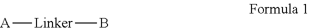
wherein A of the compound is selected from: 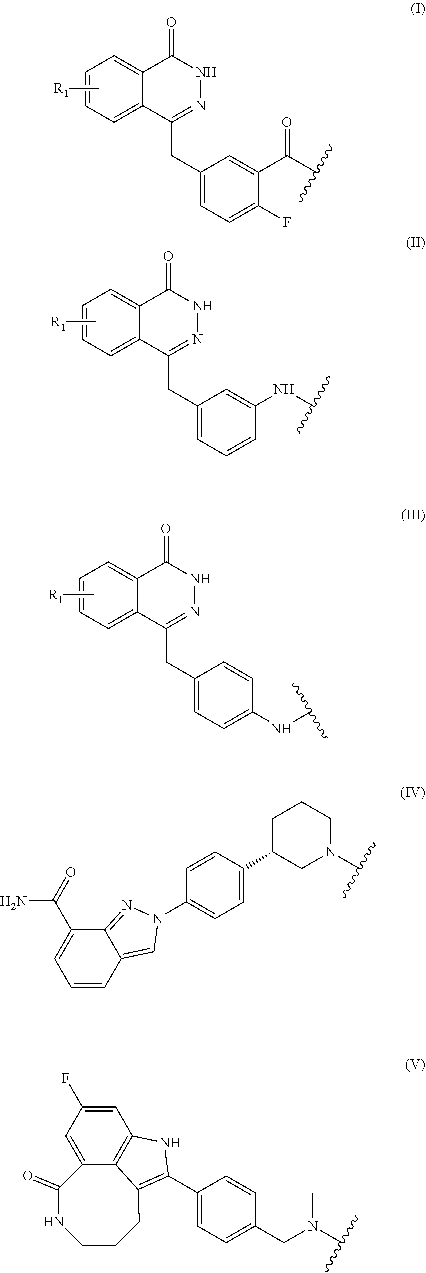
wherein R₁ is selected from H, 1-F, 2-F, 3-F, 6-F, 1-Cl, 2-Cl, 3-Cl, 6-Cl, 1-Br, 2-Br, 3-Br, 6-Br, 1-I, 2-I, 3-I, 6-I, 1-I, 2-I, 3-I, 6-I, 1-NO₂, 2-NO₂, 3-NO₂, 6-NO₂, 1-CH₃, 2-CH₃, 3-CH₃, 6-CH₃, 1-OCH₃, 2-OCH₃, 3-OCH₃, and 6-OCH₃; and
when A is selected from (I), Linker is selected from the following groups: 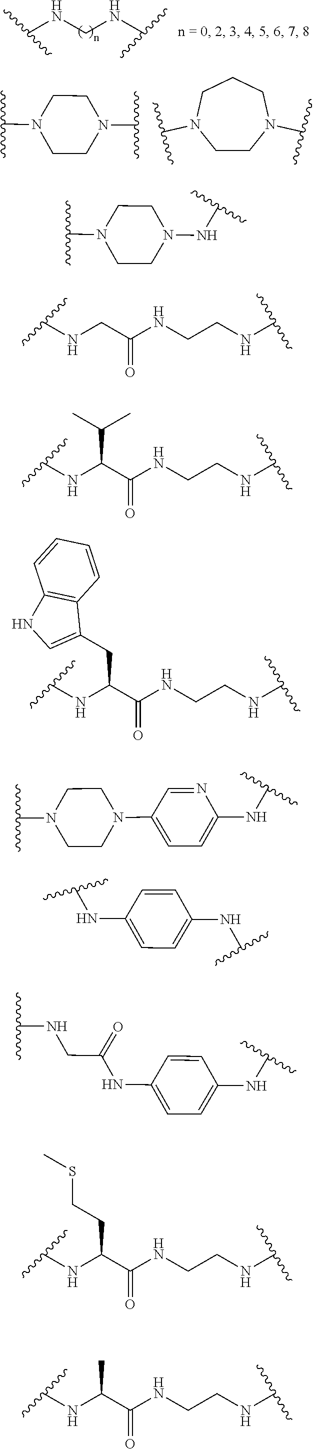 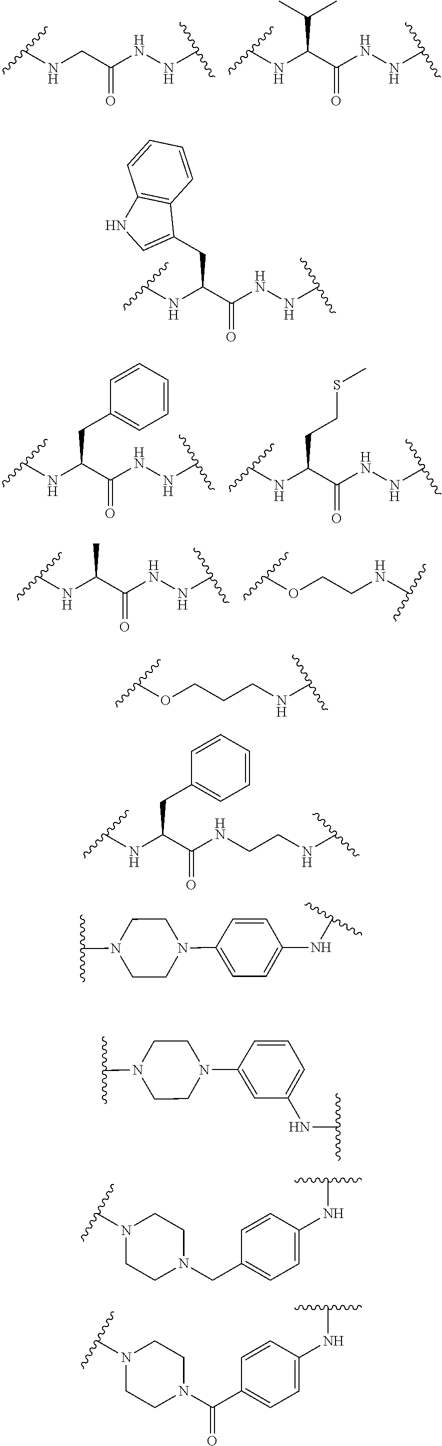 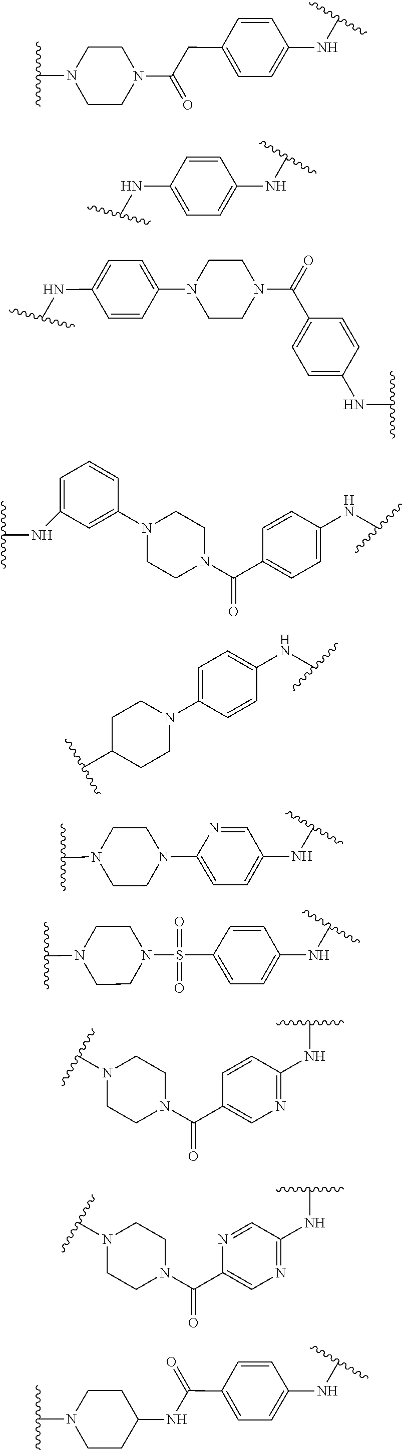 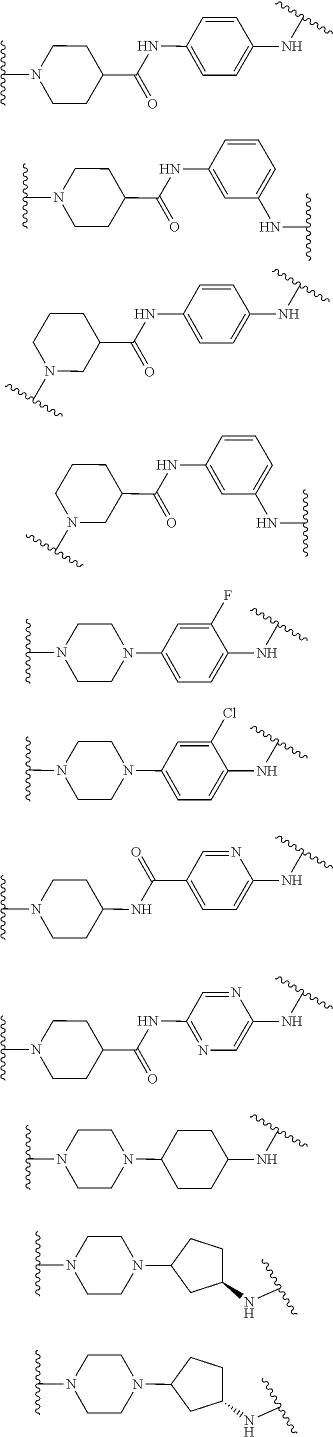 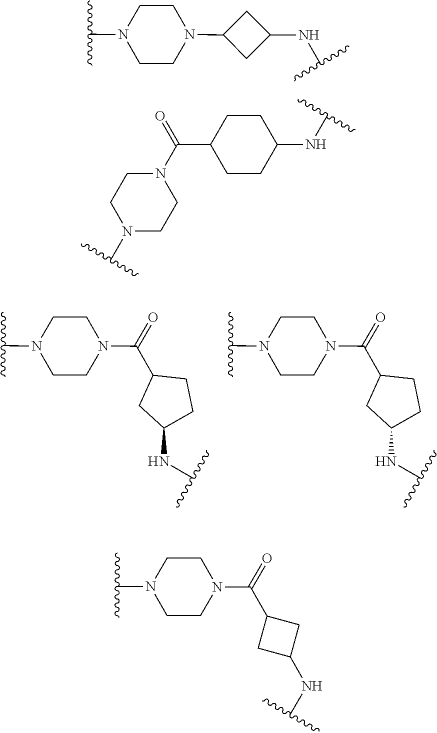 [SMILES: *NC1CC(N2CCN(*)CC2)C1 ; *NC1CCC(C(=O)N2CCN(*)CC2)CC1 ; *N[C@@H]1CCC(C(=O)N2CCN(*)CC2)C1 ; *N[C@H]1CCC(C(=O)N2CCN(*)CC2)C1 ; *NC1CC(C(=O)N2CCN(*)CC2)C1]
when A is selected from (II), (III), (IV), and (V), Linker is selected from the following groups: 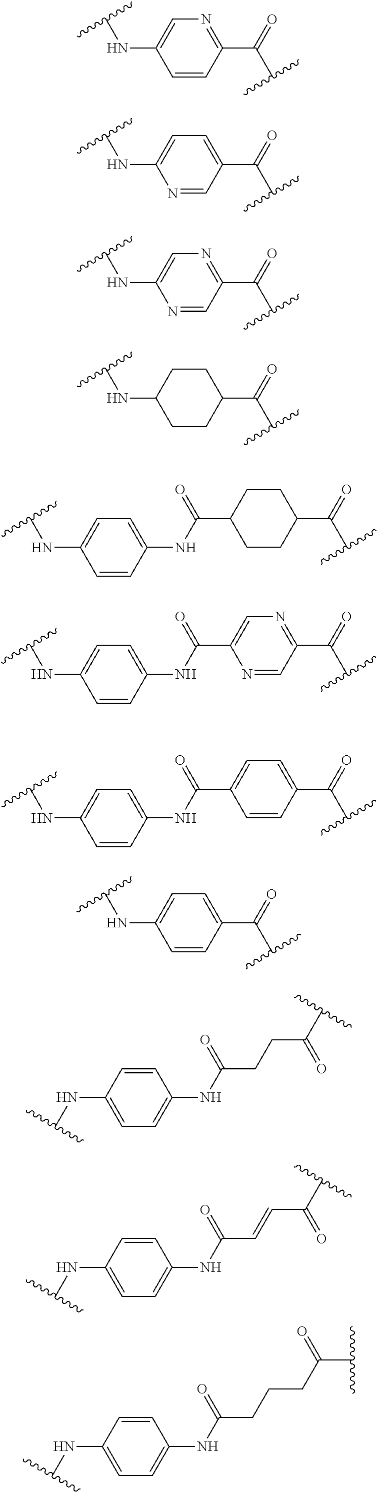
wherein B of the compound is selected from the following groups: 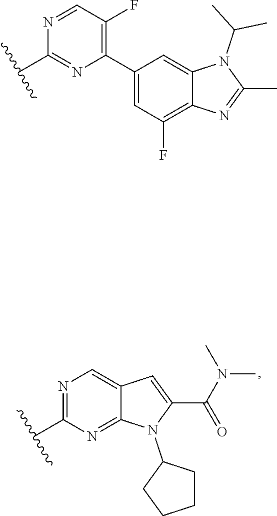 [SMILES: *c1ncc(F)c(-c2cc(F)c3nc(C)n(C(C)C)c3c2)n1 ; *c1ncc2cc(C(=O)N(C)C)n(C3CCCC3)c2n1]
and
the dual-target inhibitor as an active ingredient and a pharmaceutically acceptable carrier.
